SEQUENCE LISTING (10 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

(19) United States
(12) Patent Application Publication
(10) Pub. No.: US 2026/0092117 A1
(43) Pub. Date: Apr. 2, 2026

(54) METHODS AND COMPOSITIONS FOR CLASSIFYING AND TREATING BLADDER CANCER

(71) Applicant: Genentech, Inc., South San Francisco, CA (US)

(72) Inventors: Habib HAMIDI, Fremont, CA (US); Sanjeev MARIATHASAN, Millbrae, CA (US); Romain Francois BANCHEREAU, San Mateo, CA (US)

(21) Appl. No.: 19/170,519

(22) Filed: Apr. 4, 2025

Related U.S. Application Data
(63) Continuation of application No. PCT/US2023/075998, filed on Oct. 4, 2023.
(60) Provisional application No. 63/378,478, filed on Oct. 5, 2022.
(60) Provisional application No. 63/587,391, filed on Oct. 2, 2023.

(51) Int. Cl. C07K 16/28; A61K 39/00; A61P 35/00; C12Q 1/6886

(52) U.S. Cl. CPC C07K 16/2827; A61P 35/00; C12Q 1/6886; A61K 2039/505; C12Q 2600/106; C12Q 2600/158

(57) ABSTRACT

The invention provides methods for classifying bladder cancer (e.g., urothelial cancer (UC), e.g., locally advanced or metastatic UC); methods for treating bladder cancer in a patient, for example, by administering a treatment regimen that comprises a PD-1 axis binding antagonist (e.g., atezolizumab) to the patient. Also provided are compositions for use, kits, and articles of manufacture for use in classifying and treating bladder cancer in a patient.

### SEQUENCE LISTING

The instant application contains a Sequence Listing which has been submitted electronically in XML format and is hereby incorporated by reference in its entirety. Said XML copy, created on Mar. 31, 2025, is named 50474-274004_Sequence_Listing_3_31_25.XML and is 10,413 bytes in size.

### FIELD OF THE INVENTION

This invention relates to methods and compositions for use in classifying and treating bladder cancer (e.g., urothelial carcinoma (UC)) in a patient.

### BACKGROUND OF THE INVENTION

Cancer remains one of the deadliest threats to human health. Cancers, or malignant tumors, metastasize and grow rapidly in an uncontrolled manner, making timely detection and treatment extremely difficult. In the U.S., cancer affects nearly 1.3 million new patients each year, and is the second leading cause of death after heart disease, accounting for approximately 1 in 4 deaths. Solid tumors are responsible for most of those deaths.

Bladder cancer is the fifth-most common malignancy worldwide, with close to 400,000 newly diagnosed cases and approximately 150,000 associated deaths reported per year. Approximately 81,400 new cases of urinary bladder cancer were estimated to be diagnosed in 2020 in the US, and an estimated 17,980 people were estimated to die from the disease in 2020. Urinary bladder cancer is the fourth most common cancer in men and represents about 7% of all cancer cases. Metastatic urothelial carcinoma (mUC) represents a subgroup of this disease associated with poor outcomes, the most unmet medical need, and few effective therapies to date. The standard of care for mUC has been platinum-based chemotherapy with an overall survival of 9 to 15 months. Encouragingly, for patients who relapse on this type of therapy or patients who are ineligible to receive cisplatin, novel checkpoint inhibitors have supported improved outcomes.

Treatment with inhibitors of the PD-L1 axis pathway has resulted in significant improvement in clinical outcomes in patients with advanced UC. However, not all patients respond to PD-L1 inhibition as monotherapy. Thus, a better understanding of the molecular basis of clinical heterogeneity in patients with advanced UC is needed to inform treatment selection strategies and delineate resistance mechanisms. Moreover, improved methods of patient classification and treatment are needed.

### SUMMARY OF THE INVENTION

The present disclosure provides, inter alia, methods of classifying bladder cancer (e.g., UC, e.g., locally advanced or metastatic UC, including in the first-line (1L), second-line (2L), and later (2L+) treatment settings), methods of treating bladder cancer, and related kits, compositions for use, and uses.

In one aspect, the invention features a method of classifying a urothelial cancer (UC) in a human patient, the method comprising (a) assaying mRNA in a tumor sample from the patient to provide a transcriptional profile of the patient's tumor; and (b) assigning the patient's tumor sample into one of the following four subtypes based on the transcriptional profile of the patient's tumor: luminal, stromal, immune, or basal, thereby classifying the UC in the patient.

In another aspect, the invention features a method of treating a UC in a human patient, the method comprising: classifying the UC in the patient according to any one of the methods disclosed herein; and administering an anti-cancer therapy to the patient based on the UC subtype.

In another aspect, the invention features an anti-cancer therapy for use in treating a UC in a human patient, wherein the UC in the patient has been classified according to any one of the methods disclosed herein.

In another aspect, the invention features the use of an anti-cancer therapy in the preparation of a medicament for treating a UC in a human patient, wherein the UC in the patient has been classified according to any one of the methods disclosed herein.

In some aspects, the anti-cancer therapy includes a PD-1 axis binding antagonist (e.g., an anti-PD-L1 antibody, e.g., atezolizumab). In some aspects, the anti-cancer therapy includes atezolizumab. In some aspects, the anti-cancer therapy includes a PD-1 axis binding antagonist (e.g., atezolizumab) and one or more additional immunotherapy agents (e.g., an anti-TIGIT antibody or anti-PD-1/anti-LAG3 bispecific antibody). In some aspects, the anti-cancer therapy includes a PD-1 axis binding antagonist (e.g., atezolizumab) and one or more additional agents (e.g., a tyrosine kinase inhibitor (TKI), an FGFR3 antagonist, an anti-HER2 antibody drug conjugate (ADC), an anti-TROP2 ADC, or a combination thereof). In some aspects, the anti-cancer therapy includes a PD-1 axis binding antagonist (e.g., atezolizumab) and one or more additional agents (e.g., a TKI, a TGF-β antagonist, a chemotherapeutic agent, or a combination thereof).

In another aspect, the invention features a kit for performing any one of the methods disclosed herein. In some aspects, the kit comprises (a) reagents for assaying mRNA in a tumor sample from the patient to provide a transcriptional profile of the patient's tumor; and (b) instructions for assigning the patient's tumor sample into following four subtypes based on the transcriptional profile of the patient's tumor: luminal, stromal, immune, or basal, thereby classifying the UC.

### BRIEF DESCRIPTION OF THE DRAWINGS

FIG. 1 is a schematic diagram showing the number of patients (n) included in this study from the phase II IMvigor210, phase III IMvigor211, and phase III IMvigor010 clinical trials. ctDNA, circulating tumor DNA.

FIG. 2A is a consensus matrix depicting clusters identified by non-negative matrix factorization (NMF) clustering of patient tumors from the IMvigor010, IMvigor210, and IMvigor211 clinical trials. NMF clusters 1˜4 are shown (top, horizontal axis).

FIG. 2B is a pie chart showing the distribution of patient tumors by NMF cluster.

FIG. 2C is a bar plot showing the percentage of patient tumors by NMF cluster in the IMvigor010, IMvigor210, and IMvigor211 clinical trials.

FIG. 3A is a bar plot showing the percentage of patient tumors having the indicated tumor-infiltrating immune cell (IC) scores in each NMF cluster. PD-L1 expression was measured by immunohistochemistry (IHC). Light gray, IC0; gray, IC1; dark gray, IC2+.

FIG. 3B is a bar plot showing the percentage of patient tumors having the indicated tumor cell (TC) scores in each NMF cluster. PD-L1 expression was measured by IHC. Light gray, TC0; gray, TC1; dark gray, TC2+.

FIG. 3C is a bar plot showing the percentage of patient tumors by cancer immunotherapy (CIT) phenotype in each NMF cluster. Gray, “immune desert”; light gray, “immune excluded”; dark gray, “inflamed.”

FIG. 4A is a heatmap of genes comprised in transcriptional signatures. Samples are grouped by NMF cluster. tGE8, T-effector gene expression signature; F-TBRS, fibroblast TGF-β response signature; FAB, fatty acid biosynthesis; UGTs, UDP glucuronosyltransferase family members.

FIG. 4B is a dot plot summarizing the heatmap in FIG. 4A. Samples were aggregated by NMF cluster using the mean across samples for each gene, and the median z-score for each signature was calculated, resulting in one z-score per signature per NMF cluster.

FIG. 4C is a series of oncoprints displaying somatic alterations in NMF clusters (NMF1-4). Tumor mutational burden (TMB) is represented for individual samples as a bar plot above the oncoprint. The horizontal bar plots to the right of each oncoprint represent the number of patients with alterations for each gene.

FIGS. 5A-5C are a series of Kaplan-Meier plots of overall survival (OS) by NMF cluster of patient tumors from atezolizumab-treated patients from the IMvigor210 study (FIG. 5A), atezolizumab-treated patients from the IMvigor010 study (FIG. 5B), and observation patients from the IMvigor010 study (FIG. 5C). log rank pval, log rank p-value.

FIG. 6 is a forest plot for OS hazard ratios in patients treated with atezolizumab vs. chemotherapy in the IMvigor211 study, atezolizumab vs. observation in ctDNA-patients in the IMvigor010 study, or atezolizumab vs. observation in ctDNA+ patients in the IMvigor010 study. The OS hazard ratios for each NMF cluster are shown.

FIG. 7 is a schematic diagram showing the number of patients (n) included in this study from the phase II IMvigor210, phase III IMvigor211, phase III IMvigor010, and phase III IMvigor130 clinical trials. ctDNA, circulating tumor DNA; atezo, atezolizumab; chemo, chemotherapy.

FIG. 8A is a line chart representing the cophenetic coefficient analysis across NMF 2-8 splits.

FIG. 8B is a consensus matrix for k=4 depicting clusters identified by non-negative matrix factorization (NMF) clustering of patient tumors from the clinical trials.

FIG. 8C is pie chart representing the distribution of NMF subtypes across trials.

FIG. 8D is a bar chart representing the distribution of NMF subtypes across trials.

FIGS. 9A-9C are a series of Kaplan-Meier curves representing OS probability, split by NMF subtypes in arms combined (FIG. 9A), atezolizumab-treated patients (FIG. 9B) or SOC-treated patients (FIG. 9C). SOC, standard of care; obsrv, observation; log rank pval, log rank p-value.

FIG. 9D is a series of Kaplan-Meier curves representing OS probability in each NMF subtype, separated by treatment arm (dark gray: atezolizumab-containing; light gray: standard-of-care). pval, p-value; HR, hazard ratio.

FIG. 9E is a forest plot summarizing hazard ratios (HR), confidence intervals (CI), p-values (Pval) and median OS for curves shown in FIG. 9D. w/Atezo, with atezolizumab.

FIG. 10A is a bar chart representing the distribution of PD-L1 expression on immune cells by NMF subtype (IC0: <1%; IC1: <5%; IC2+: ≥5%) (light gray, IC0; gray, IC1; dark gray, IC2+). IC, immune cell.

FIG. 10B is a bar chart representing the distribution of PD-L1 expression on tumor cells by NMF subtype (TC0: <1%; IC1: <5%; IC2+: ≥5%) (light gray, TC0; gray, TC1; dark gray, TC2+). TC, tumor cell.

FIG. 10C is a bar chart representing the distribution of cancer immunotherapy (CIT) phenotype (CD8+ T cell infiltration pattern) by NMF subtype (gray, “immune desert”; light gray, “immune excluded”; dark gray, “inflamed”). pheno, phenotype.

FIG. 10D is a box plot representing tumor mutational burden (TMB) by NMF subtype. Significance is assessed by Pairwise Wilcoxon Rank Sum Test with Benjamini-Hochberg multiple testing correction (*: p<0.05; **: p<0.01; ***: p<0.001). Mb, million bases.

FIG. 10E is a series of bar charts representing the enrichment of liver metastases, specimen type (metastasis vs. primary), lymph node origin, sampling methodology (biopsy vs. trans urethral resection of bladder tumor (TURBT) vs. resection) and urinary tract location (upper vs. lower) by NMF subtype.

FIG. 10F is a heatmap representing selected transcriptional signatures across NMF subtypes. Data represent the z-scored log 2 (transcript-per-million (TPM)+1) transformed counts. Samples are ordered by NMF subtype and CIT phenotype. Genes are hierarchically clustered using Euclidean distance. ECM, extracellular matrix; F-TBRS, fibroblast TGF-β response signature; FAB, fatty acid biosynthesis; UGTs, UDP glucuronosyltransferase family members; IC, immune cell; TC, tumor cell.

FIG. 10G is a bar chart representing the distribution of luminal/basal ratio categories across NMF subtypes. Luminal and basal signatures were dichotomized as high or low based on the median expression across the entire dataset. Samples were then categorized as LumHigh/BasLow, LumLow/BasLow, LumHigh/BasHigh and LumLow/BasHigh. Statistical significance was assessed by the Chi-square test.

FIG. 10H is a box plot representing the basal/luminal ratio on a continuous scale by NMF subtype. Statistical significance was assessed by the Kruskal-Wallis rank sum test.

FIG. 10I is a dot map of transcriptional signatures from FIG. 10F aggregated by NMF subtype and clinical trial. The color scale represents the mean z-score for each group.

FIG. 10J is a series of box plots depicting cell population-specific enrichment of different patient clusters determined by xCell. CD8pos, CD8-positive; DC, dendritic cell.

FIG. 10K is a heatmap representing cell population enrichment based on xCell deconvolution. Data represent z-scored xCell enrichment score. Samples are ordered by NMF subtype and CIT phenotype. Genes are hierarchically clustered on the dataset aggregated by NMF subtype (right panel) using Euclidean distance.

FIG. 10L is a heatmap representing hematoxylin and eosin (H&E)-based digital pathology-derived human interpretable features (HIFs) significantly modulated between NMF subtypes across IMvigor clinical trials. Data represent z-scored HIF enrichment across the sampled population. Samples are ordered by NMF subtype and clinical trial.

FIG. 10M is a series of box plot depicting representative human interpretable features by NMF subtype, for training (dark gray, IMvigor210/211/010) and test (light gray, IMvigor130) sets.

FIG. 11A is a pie chart representing the distribution of Lund subtypes across the clinical trials. UroA, urobasal A; GU, genomically unstable; UroB, urobasal B; SCCL, squamous cell carcinoma-like.

FIG. 11B is a bar chart representing the distribution of Lund subtypes within each NMF subtype.

FIG. 11C is a forest plot representing the clinical benefit of atezolizumab-containing arms vs. SOC for each Lund subtype.

FIG. 11D is a pie chart representing the distribution of the Cancer Genome Atlas (TCGA) subtypes across the clinical trials.

FIG. 11E is a bar chart representing the distribution of TCGA subtypes within each NMF subtype.

FIG. 11F is a forest plot representing the clinical benefit of atezolizumab-containing arms vs. SOC for each TCGA subtype.

FIG. 12A is an oncoprint of the genes somatically altered in at least 5% of patients. Tumor mutational burden (TMB) is represented for individual samples as a bar plot above the oncoprint. The horizontal bar plot to the right of the oncoprint represents the number of patients with alterations for each gene.

FIG. 12B is a series of pie charts representing somatic alteration prevalence by NMF subtype (somatically altered samples are represented in dark gray). P-values are calculated using the Chi-square test.

FIG. 12C is a heatmap representing associations between somatic alterations and OS by treatment arm. White dots represent a significant p-value for the Cox proportional hazard model.

FIG. 13A is a series of Kaplan-Meier curves representing the probability of OS, split by treatment arm and PD-L1 IC expression (interrupted lines: IC01, IC<5%; continuous lines: IC23, IC≥5%) in each NMF subtype (dark gray, atezolizumab-containing arm; light gray, standard-of-treatment arm).

FIG. 13B is a heatmap representing the associations between transcriptional signatures and OS by treatment arm. White dots represent a significant p-value for the Cox proportional hazard model.

FIG. 13C is a series of Kaplan-Meier curves representing the probability of OS based on the expression of the myeloid, plasma cell and neutrophil signatures. Signatures were dichotomized as high (interrupted lines) or low (continuous lines) based on the median expression across the complete dataset (dark gray, atezolizumab-containing arm; light gray, standard-of-treatment arm).

FIG. 14A is a series of heatmaps representing chemoattractants differentially expressed between NMF subtypes. Data represent the z-scored log₂ (TPM+1) transformed counts.

FIG. 14B is a pair of bar charts of neutrophil score by NMF subtype (left) and luminal/basal signatures (right), determined by pathology in IMvigor210 and IMvigor010 (light gray, low neutrophil score; dark gray, high neutrophil score).

FIG. 14C is a Uniform Manifold Approximation and Projection (UMAP) of the epithelial compartment in twelve UC patients profiled by single-cell RNAseq in two independent studies. The gray interrupted shape highlights two tumors (Tumor5 and humanN_171) enriched for basal markers.

FIG. 14D is a series of violin plots representing the expression of basal markers KRT5 and KRT6A and granulocyte chemoattractants CXCL1 and CXCL2 in clusters from FIG. 14C.

FIG. 15 is a diagram summarizing UC molecular subtypes, including RNA profiles, enriched somatic alterations, PD-L1 IC expression, CD8+ T cell infiltration patterns, and proposed targets for combination therapy.

### DETAILED DESCRIPTION OF THE INVENTION

The present invention provides diagnostic and therapeutic methods and compositions for cancer, for example, bladder cancer (e.g., UC, e.g., locally advanced or metastatic UC, including in the first-line (1L), second-line (2L), and later (2L+) treatment settings). The invention is based, at least in part, on the discovery that the methods of classification described herein identify patient subgroups that have unexpectedly favorable response to anti-cancer therapies, including anti-cancer therapies that include a PD-1 axis binding antagonist (e.g., an anti-PD-L1 antibody, e.g., atezolizumab), as shown in Example 1. Moreover, Example 1 demonstrates that the methods of classification herein are expected to be effective for identifying patient subgroups for a PD-1 axis binding antagonist (e.g., an anti-PD-L1 antibody, e.g., atezolizumab) in combination with other anti-cancer therapies, such as a tyrosine kinase inhibitor (TKI), an FGFR3 antagonist, an anti-HER2 antibody drug conjugate (ADC), an anti-TROP2 ADC, or a combination thereof. Based on these data, it is expected that the methods of classification described herein can also identify patient subgroups with favorable response to a PD-1 axis binding antagonist (e.g., an anti-PD-L1 antibody, e.g., atezolizumab), alone or in combination with other anti-cancer therapies, e.g., anti-cancer therapies including an immunotherapy agent, a cytotoxic agent, a growth inhibitory agent, a stromal inhibitor, a metabolism inhibitor, a complement antagonist, a radiation therapy agent, an anti-angiogenic agent, or a combination thereof.

### I. Definitions

The term “anti-cancer therapy” refers to a therapy useful in treating cancer. An anti-cancer therapy may include a treatment regimen with one or more anti-cancer therapeutic agents. Examples of anti-cancer therapeutic agents include, but are limited to, an immunotherapy agent (e.g., a PD-1 axis binding antagonist), a cytotoxic agent, a growth inhibitory agent, a stromal inhibitor, a metabolism inhibitor, a complement antagonist, a radiation therapy agent, an anti-angiogenic agent, an antibody-drug conjugate (ADC), and other agents to treat cancer. Combinations thereof are also included in the invention.

An “immunoconjugate” or “antibody drug conjugate” or “ADC” is an antibody conjugated to one or more heterologous molecule(s), including but not limited to a cytotoxic agent. Exemplary, non-limiting antibody drug conjugates include anti-HER2 antibody drug conjugates (anti-HER2 ADC) (e.g., trastuzumab emtansine (T-DM1, ado-trastuzumab emtansine, KADCYLA®, Genentech), trastuzumab deruxtecan (DS-8201a, T-DXd, ENHERTU®, Gilead), trastuzumab duocarmazine (SYD985, Byondis), A166, XMT-1522, MEDI-4276, ARX788, RC48-ADC, BAT8001, PF-06804103) and anti-TROP2 antibody drug conjugates (anti-TROP2 ADC) (e.g., sacituzumab govitecan (TRODELVY®, Gilead), datopotamab deruxtecan (Dato-DXd, DS-1062a, Daiichi Sankyo, AstraZeneca), BAT8003 (Biothera)). Exemplary, non-limiting antibody drug conjugates are described in Criscitiello et al. J Hematol Oncol. 14:20 (2021). The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

A “stromal inhibitor” refers to any molecule that partially or fully blocks, inhibits, or neutralizes a biological activity and/or function of a gene or gene product associated with stroma (e.g., tumor-associated stroma). In some embodiments, the stromal inhibitor partially or fully blocks, inhibits, or neutralizes a biological activity and/or function of a gene or gene product associated with fibrotic tumors. In some embodiments, treatment with a stromal inhibitor results in the reduction of stroma, thereby resulting in an increased activity of an immunotherapy; for example, by increasing the ability of activating immune cells (e.g., proinflammatory cells) to infiltrate a fibrotic tissue (e.g., a fibrotic tumor). Targets for stromal gene antagonists are known in the art; for example, see Turley et al., Nature Reviews Immunology 15:669-682, 2015 and Rosenbloom et al., Biochimica et Biophysica Acta 1832:1088-1103, 2013. In some embodiments, the stromal inhibitor is a transforming growth factor beta (TGF-β), podoplanin (PDPN), leukocyte-associated immunoglobulin-like receptor 1 (LAIR1), SMAD, anaplastic lymphoma kinase (ALK), connective tissue growth factor (CTGF/CCN2), endothelial-1 (ET-1), AP-1, interleukin (IL)-13, lysyl oxidase homolog 2 (LOXL2), endoglin (CD105), fibroblast activation protein (FAP), vascular cell adhesion protein 1 (CD106), thymocyte antigen 1 (THY1), beta 1 integrin (CD29), platelet-derived growth factor (PDGF), PDGF receptor A (PDGFRα), PDGF receptor B (PDGFRβ), vimentin, smooth muscle actin alpha (ACTA2), desmin, endosialin (CD248), or S100 calcium-binding protein A4 (S100A4) antagonist.

A “TGF-β antagonist” or a “TGF-β inhibitor,” as used interchangeably herein, refers to any molecule that decreases, blocks, inhibits, abrogates or interferes with signal transduction resulting from the interaction of TGF-β with one or more of its interaction partners, such as a TGF-β cellular receptor. In some embodiments, a “TGF-binding antagonist” is a molecule that inhibits the binding of TGF-β to its binding partners. In some embodiments, the TGF-β antagonist inhibits the activation of TGF-β. In some embodiments, the TGF-β antagonist includes an anti-TGF-β antibody, antigen binding fragments thereof, an immunoadhesin, a fusion protein, an oligopeptide, and other molecules that decrease, block, inhibit, abrogate or interfere with signal transduction resulting from the interaction of TGF-β with one or more of its interaction partners. In some embodiments, the TGF-β antagonist is a polypeptide, a small molecule, or a nucleic acid. In some embodiments, the TGF-β antagonist (e.g., the TGF-β binding antagonist) inhibits TGF-β1, TGF-β2, and/or TGF-β3. In some embodiments, the TGF-β antagonist (e.g., the TGF-β binding antagonist) inhibits TGF-β receptor-1 (TGFBR1), TGF-β receptor-2 (TGFBR2), and/or TGF-β receptor-3 (TGFBR3).

The terms “anti-TGF-β antibody” and “an antibody that binds to TGF-β” refer to an antibody that is capable of binding TGF-β with sufficient affinity such that the antibody is useful as a diagnostic and/or therapeutic agent in targeting TGF-β. In one embodiment, the extent of binding of an anti-TGF-β antibody to an unrelated, non-TGF-β protein is less than about 10% of the binding of the antibody to TGF-β as measured, for example, by a radioimmunoassay (RIA). In certain embodiments, an anti-TGF-β antibody binds to an epitope of TGF-β that is conserved among TGF-β from different species. In some embodiments, the anti-TGF-β antibody inhibits TGF-β1, TGF-β2, and/or TGF-β3. In some embodiments, the anti-TGF-β antibody inhibits TGF-β1, TGF-β2, and TGF-β3. In some embodiments, the anti-TGF-β antibody is a pan-specific anti-TGF-β antibody. In some embodiments, the anti-TGF-β antibody may be any anti-TGF-β antibody disclosed in, for example, U.S. Pat. No. 5,571,714 or in International Patent Application Nos. WO 92/00330, WO 92/08480, WO 95/26203, WO 97/13844, WO 00/066631, WO 05/097832, WO 06/086469, WO 05/010049, WO 06/116002, WO 07/076391, WO 12/167143, WO 13/134365, WO 14/164709, or WO 16/201282, each of which is incorporated herein by reference in its entirety. In particular embodiments, the anti-TGF-β antibody is fresolimumab, metelimumab, lerdelimumab, 1D11, 2G7, or a derivative thereof.

A “metabolism inhibitor” refers to any molecule that disrupts metabolism (e.g., basal metabolism), metabolic pathways and/or levels of metabolites of a cell (e.g., a cancer cell), either directly or indirectly. In some embodiments, a metabolism inhibitor may stimulate any change in metabolism (e.g., basal metabolism), metabolic pathways, and/or levels of metabolites of a cell. Metabolic pathways can include, but are not limited to, amino acid catabolismo cellular respiration, oxidative phosphorylation (OXPHOS), glycolysis, fatty acid oxidation, fatty acid metabolism, electron transport chain (ETC) complex I activity, ETC complex II activity, ETC complex III activity, ETC complex IV activity, the tricarboxylic acid (TCA) cycle, amino acid uptake, any catabolic pathway, any anabolic pathway, any amphibolic pathway, catabolismanabolism, gluconeogenesis, glycogenolysis, glycogenesis, the urea cycle, aminotransferase pathways, acetyl-CoA synthesis pathways, pentose phosphate pathway, fructolysis, galactolysis, glycosylation, beta oxidation, fatty acid degradation, fatty acid synthesis, steroid metabolism, sphingolipid metabolism, eicosanoid metabolism, ketosis, reverse cholesterol transport, glutamine/glutamate catabolismasparagine/aspartate catabolismalanine catabolismarginine, ornithine and proline catabolismserine catabolismthreonine catabolismglycine catabolismcysteine catabolismmethionine catabolismleucine, isoleucine and valine catabolismphenylalanine and tyrosine catabolismlysine catabolismhistidine catabolismtryptophan catabolismor any combination thereof. In some embodiments, the metabolism inhibitor is a proprotein convertase subtilisin/kexin type 9 serine protease (PCSK9) inhibitor (e.g., an anti-PCSK9 antibody, e.g., alirocumab or evolocumab), fatty acid synthase (FAS) inhibitor (e.g., cerulenin, C75, isoniazid, or orlistat (tetrahydrolipstatin)), carnitine palmitoyltransferase-1 (CPT-1) inhibitor (e.g., etomoxir), GLUT4 inhibitor (e.g., ritonavir, indinavir, or analogs or derivatives thereof), or OXPHOS inhibitor (e.g., compounds within the biguanide class of drugs, e.g., metformin, phenformin, buformin, and pharmaceutically acceptable salts thereof).

An “angiogenesis inhibitor” or “anti-angiogenic agent” or “anti-angiogenesis agent,” as used interchangeably herein, refers to a small molecular weight substance (including tyrosine kinase inhibitors), a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or the VEGF-A receptor (e.g., KDR receptor or Flt-1 receptor), anti-PDGFR inhibitors such as GLEEVEC™ (imatinib mesylate). Anti-angiogenesis agents also include native angiogenesis inhibitors, e.g., angiostatin, endostatin, etc. See, for example, Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12): 1359-1364 (1999); Tonini et al., Oncogene, 22:6549-6556 (2003) and Sato Int. J. Clin. Oncol., 8:200-206 (2003). In some examples, the angiogenesis inhibitor is an anti-VEGF antibody or an antigen-binding fragment thereof, e.g., bevacizumab.

A “tyrosine kinase inhibitor” is an antagonist molecule which inhibits to some extent tyrosine kinase activity of a tyrosine kinase such as an EGFR receptor or an FGFR3 receptor.

As used herein, the term “FGFR3 antagonist” and “FGFR3 inhibitor” refers to any FGFR3 antagonist that is currently known in the art or that will be identified in the future, and includes any chemical entity that, upon administration to a patient, results in inhibition of a biological activity associated with activation of FGFR3 in the patient, including any of the downstream biological effects otherwise resulting from the binding to FGFR3 of its natural ligand. Such FGFR3 antagonists include any agent that can block FGFR3 activation or any of the downstream biological effects of FGFR3 activation that are relevant to treating cancer in a patient. Such an antagonist can act by binding directly to the intracellular domain of the receptor and inhibiting its kinase activity. Alternatively, such an antagonist can act by occupying the ligand binding site or a portion thereof of the FGFR3 receptor, thereby making the receptor inaccessible to its natural ligand so that its normal biological activity is prevented or reduced. Alternatively, such an antagonist can act by modulating the dimerization of FGFR3 polypeptides, or interaction of FGFR3 polypeptide with other proteins, or enhance ubiquitination and endocytotic degradation of FGFR3. FGFR3 antagonists include but are not limited to small molecule inhibitors, antibodies or antibody fragments, antisense constructs, small inhibitory RNAs (i.e., RNA interference by dsRNA; RNAi), and ribozymes. In some embodiments, the FGFR3 antagonist is a small molecule or an antibody that binds specifically to human FGFR3. Exemplary FGFR3 antagonist antibodies are described, for example, in U.S. Pat. No. 8,410,250, which is incorporated herein by reference in its entirety. For example, U.S. Pat. No. 8,410,250 describes the FGFR3 antagonist antibody clones 184.6, 184.6.1, and 184.6.1N54S (these clones are also referred to as “R3 Mab”).

The term “immunotherapy agent” refers to the use of a therapeutic agent that modulates an immune response. Exemplary, non-limiting immunotherapy agents include a PD-1 axis binding antagonist, a CTLA-4 antagonist (e.g., an anti-CTLA-4 antibody (e.g., ipilimumab)), a TIGIT antagonist (e.g., an anti-TIGIT antibody (e.g., tiragolumab)), PD1-IL2v (a fusion of an anti-PD-1 antibody and modified IL-2), PD1-LAG3, IL-15, anti-CCR8 (e.g., an anti-CCR8 antibody, e.g., FPA157), FAP-4-1BBL (fibroblast activation protein-targeted 4-1BBL agonist), or a combination thereof. In some examples, the immunotherapy agent is an immune checkpoint inhibitor. In some examples, the immunotherapy agent is a CD28, OX40, GITR, CD137, CD27, IC0S, HVEM, NKG2D, MICA, or 2B4 agonist or a CTLA-4, PD-1 axis, TIM-3, BTLA, VISTA, LAG-3, B7H4, CD96, TIGIT, or CD226 antagonist. Other particular immunotherapy agents include anti-TIGIT antibodies (e.g., tiragolumab) and antigen-binding fragments thereof, anti-CTLA-4 antibodies or antigen-binding fragments thereof, anti-CD27 antibodies or antigen-binding fragments thereof, anti-CD30 antibodies or antigen-binding fragments thereof, anti-CD40 antibodies or antigen-binding fragments thereof, anti-4-1BB antibodies or antigen-binding fragments thereof, anti-GITR antibodies or antigen-binding fragments thereof, anti-OX40 antibodies or antigen-binding fragments thereof, anti-TRAILR1 antibodies or antigen-binding fragments thereof, anti-TRAILR2 antibodies or antigen-binding fragments thereof, anti-TWEAK antibodies or antigen-binding fragments thereof, anti-TWEAKR antibodies or antigen-binding fragments thereof, anti-BRAF antibodies or antigen-binding fragments thereof, anti-MEK antibodies or antigen-binding fragments thereof, anti-CD33 antibodies or antigen-binding fragments thereof, anti-CD20 antibodies or antigen-binding fragments thereof, anti-CD52 antibodies or antigen-binding fragments thereof, anti-A33 antibodies or antigen-binding fragments thereof, anti-GD3 antibodies or antigen-binding fragments thereof, anti-PSMA antibodies or antigen-binding fragments thereof, anti-Ceacan 1 antibodies or antigen-binding fragments thereof, anti-Galedin 9 antibodies or antigen-binding fragments thereof, anti-HVEM antibodies or antigen-binding fragments thereof, anti-VISTA antibodies or antigen-binding fragments thereof, anti-B7 H4 antibodies or antigen-binding fragments thereof, anti-HHLA2 antibodies or antigen-binding fragments thereof, anti-CD155 antibodies or antigen-binding fragments thereof, anti-CD80 antibodies or antigen-binding fragments thereof, anti-BTLA antibodies or antigen-binding fragments thereof, anti-CD160 antibodies or antigen-binding fragments thereof, anti-CD28 antibodies or antigen-binding fragments thereof, anti-CD226 antibodies or antigen-binding fragments thereof, anti-CEACAM1 antibodies or antigen-binding fragments thereof, anti-TIM3 antibodies or antigen-binding fragments thereof, anti-CD96 antibodies or antigen-binding fragments thereof, anti-CD70 antibodies or antigen-binding fragments thereof, anti-CD27 antibodies or antigen-binding fragments thereof, anti-LIGHT antibodies or antigen-binding fragments thereof, anti-CD137 antibodies or antigen-binding fragments thereof, anti-DR4 antibodies or antigen-binding fragments thereof, anti-CR5 antibodies or antigen-binding fragments thereof, anti-FAS antibodies or antigen-binding fragments thereof, anti-CD95 antibodies or antigen-binding fragments thereof, anti-TRAIL antibodies or antigen-binding fragments thereof, anti-DR6 antibodies or antigen-binding fragments thereof, anti-EDAR antibodies or antigen-binding fragments thereof, anti-NGFR antibodies or antigen-binding fragments thereof, anti-OPG antibodies or antigen-binding fragments thereof, anti-RANKL antibodies or antigen-binding fragments thereof, anti-LTBR antibodies or antigen-binding fragments thereof, anti-BCMA antibodies or antigen-binding fragments thereof, anti-TACI antibodies or antigen-binding fragments thereof, anti-BAFFR antibodies or antigen-binding fragments thereof, anti-EDAR2 antibodies or antigen-binding fragments thereof, anti-TROY antibodies or antigen-binding fragments thereof, and anti-RELT antibodies or antigen-binding fragments thereof.

The terms “programmed death ligand 1” and “PD-L1” refer herein to native sequence human PD-L1 polypeptide. Native sequence PD-L1 polypeptides are provided under UniProt Accession No. Q9NZQ7. For example, the native sequence PD-L1 may have the amino acid sequence as set forth in UniProt Accession No. Q9NZQ7-1 (isoform 1). In another example, the native sequence PD-L1 may have the amino acid sequence as set forth in UniProt Accession No. Q9NZQ7-2 (isoform 2). In yet another example, the native sequence PD-L1 may have the amino acid sequence as set forth in UniProt Accession No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In some embodiments, the bladder cancer is urothelial bladder cancer (e.g., transitional cell carcinoma (TCC) or urothelial carcinoma (UC), non-muscle invasive bladder cancer, muscle-invasive bladder cancer (MIBC), and metastatic bladder cancer) and non-urothelial bladder cancer. In one instance, the cancer is urothelial carcinoma (UC), e.g., a locally advanced or metastatic UC. The cancer may be locally advanced or metastatic. In some instances, the cancer is locally advanced. In other instances, the cancer is metastatic. In some instances, the cancer may be unresectable (e.g., unresectable locally advanced or metastatic cancer).

As used herein, “urothelial carcinoma” and “UC” refer to a type of cancer that typically occurs in the urinary system, and includes muscle-invasive bladder cancer (MIBC) and muscle-invasive urinary tract urothelial cancer (UTUC). UC is also referred to in the art as transitional cell carcinoma (TCC).

The term “ineligible for treatment with a platinum-based chemotherapy” or “unfit for treatment with a platinum-based chemotherapy” means that the subject is ineligible or unfit for treatment with a platinum-based chemotherapy, either in the attending clinician's judgment or according to standardized criteria for eligibility for platinum-based chemotherapy that are known in the art. For example, cisplatin ineligibility may be defined by any one of the following criteria: (i) impaired renal function (glomerular filtration rate (GFR)<60 mL/min); GFR may be assessed by direct measurement (i.e., creatinine clearance or ethyldediaminetetra-acetate) or, if not available, by calculation from serum/plasma creatinine (Cockcroft Gault formula); (ii) a hearing loss (measured by audiometry) of 25 dB at two contiguous frequencies; (iii) Grade 2 or greater peripheral neuropathy (i.e., sensory alteration or parasthesis including tingling); and (iv) ECOG Performance Status of 2.

As used herein, “cluster” or “subtype,” as used interchangeably herein, refers to a subtype of a cancer (e.g., bladder cancer (e.g., UC, e.g., locally advanced or metastatic UC)) that is defined, e.g., transcriptionally (e.g., as assessed by RNA-seq or other techniques described herein) and/or by evaluation of somatic alterations. Cluster analysis can be used to identify subtypes of cancer by clustering samples (e.g., tumor samples) from patients having similar gene expression patterns and to find groups of genes that have similar expression profiles across different samples. A patient's sample (e.g., tumor sample) can be assigned into a cluster as described herein. In some examples, clusters are identified by non-negative matrix factorization (NMF); however, other clustering approaches are described herein and known in the art. In some examples, a patient's tumor sample is assigned into one of the following four subtypes based on the transcriptional profile of the patient's tumor: (1) luminal; (2) stromal; (3) immune; and (4) basal. A patient's tumor sample may be assigned into a cluster as described herein using methods described herein, e.g., using a classifier as described herein (e.g., the set of genes set forth in Table 1 or a subset thereof).

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis antagonist and one or more additional therapeutic agents). Treating herein includes, inter alia, adjuvant therapy, neoadjuvant therapy, non-metastatic cancer therapy (e.g., locally advanced cancer therapy), and metastatic cancer therapy. The treatment may be first-line (also referred to as “1L”) treatment (e.g., the patient may be previously untreated or not have received prior systemic therapy), second-line (also referred to as “2L”), or later (2L+) treatment (e.g., third-line or fourth-line treatment). In some examples, the treatment may be first-line treatment (e.g., the patient may be previously untreated or not have received prior systemic therapy). In some examples, the treatment may be 2L or later (2L+) treatment. In some examples, the treatment is adjuvant therapy. In other examples, the treatment is neoadjuvant therapy.

Herein, an “effective amount” refers to the amount of a therapeutic agent (e.g., a PD-1 axis binding antagonist (e.g., atezolizumab) or a combination of therapeutic agents (e.g., a PD-1 axis antagonist and one or more additional therapeutic agents), that achieves a therapeutic result. In some examples, the effective amount of a therapeutic agent or a combination of therapeutic agents is the amount of the agent or of the combination of agents that achieves a clinical endpoint of improved overall response rate (ORR), a complete response (CR), a pathological complete response (pCR), a partial response (PR), improved survival (e.g., disease-free survival (DFS), progression-free survival (PFS) and/or overall survival (OS)), and/or improved duration of response (DOR). Improvement (e.g., in terms of response rate (e.g., ORR, CR, and/or PR), survival (e.g., PFS and/or OS), or DOR) may be relative to a suitable reference, for example, observation or a reference treatment (e.g., treatment that does not include the PD-1 axis binding antagonist (e.g., treatment with placebo)). In some instances, improvement (e.g., in terms of response rate (e.g., ORR, CR, and/or PR), survival (e.g., DFS, DSS, distant metastasis-free survival, PFS, and/or OS), DOR, and/or improved time to deterioration of function and QoL) may be relative to observation. In some instances, treatment with an anti-cancer therapy that includes atezolizumab may be compared with a reference treatment which is treatment with chemotherapy (e.g., vinflunine, paclitaxel, or docetaxel).

As used herein, “complete response” and “CR” refers to disappearance of the cancer. In some examples, tumor response is assessed according to RECIST v1.1. For example, CR may be the disappearance of all target lesions and non-target lesions and (if applicable) normalization of tumor marker level or reduction in short axis of any pathological lymph nodes to <10 mm.

As used herein, “partial response” and “PR” refers to at least a 30% decrease in the sum of the longest diameters (SLD) of target lesions, taking as reference the baseline SLD prior to treatment. In some examples, tumor response is assessed according to RECIST v1.1. For example, PR may be a ≥30% decrease in the sum of diameters (SoD) of target lesions (taking as reference the baseline SoD) or persistence of ≥1 non-target lesions(s) and/or (if applicable) maintenance of tumor marker level above the normal limits. In some examples, the SoD may be of the longest diameters for non-nodal lesions, and the short axis for nodal lesions.

As used herein, “disease progression,” “progressive disease,” and “PD” refers to an increase in the size or number of target lesions. For example, PD may be a ≥20% relative increase in the sum of diameters (SoD) of all target lesions, taking as reference the smallest SoD on study, including baseline, and an absolute increase of ≥5 mm; ≥1 new lesion(s); and/or unequivocal progression of existing non-target lesions. In some examples, the SoD may be of the longest diameters for non-nodal lesions, and the short axis for nodal lesions.

As used herein, “overall response rate,” “objective response rate,” and “ORR” refer interchangeably to the sum of CR rate and PR rate. For example, ORR may refer to the percentage of participants with a documented CR or PR.

As used herein, “progression-free survival” and “PFS” refer to the length of time during and after treatment during which the cancer does not get worse. PFS may include the amount of time patients have experienced a CR or a PR, as well as the amount of time patients have experienced stable disease. For example, PFS may be the time from randomization to PD, as determined by the investigator per RECIST v1.1, or death from any cause, whichever occurred first.

As used herein, “overall survival” and “OS” refer to the length of time from either the date of diagnosis or the start of treatment for a disease (e.g., cancer) that the patient is still alive. For example, OS may be the time from randomization to death due to any cause.

As used herein, the term “duration of response” and “DOR” refer to a length of time from documentation of a tumor response until disease progression or death from any cause, whichever occurs first. For example, DOR may be the time from the first occurrence of CR/PR to PD as determined by the investigator per RECIST v1.1, or death from any cause, whichever occurred first.

As used herein, the term “chemotherapeutic agent” refers to a compound useful in the treatment of cancer, such as bladder cancer (e.g., UC, e.g., a locally advanced or metastatic UC). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl) methoxy]phenyl]-6 [5 [[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), JAVLOR® (vinflunine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy) quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “small molecule” refers to any molecule with a molecular weight of about 2000 daltons or less, preferably of about 500 daltons or less. In some instances, a small molecule is any molecule with a molecular weight of 2000 daltons or less, preferably of 500 daltons or less.

The term “patient” refers to a human patient. For example, the patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).
   Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and an immunotherapy agent (e.g., an anti-TIGIT antibody or an anti-PD-1/anti-LAG3 bispecific antibody). As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3-week cycle. The term “detection” includes any means of detecting, including direct and indirect detection.

The term “biomarker” as used herein refers to an indicator, e.g., predictive, diagnostic, and/or prognostic, which can be detected in a sample, for example, a cluster, gene (e.g., PD-L1), an alteration (e.g., a somatic alteration), or ctDNA disclosed herein. The biomarker may serve as an indicator of a particular subtype of a disease or disorder (e.g., cancer) characterized by certain, molecular, pathological, histological, and/or clinical features. Biomarkers include, but are not limited to, clusters, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers. In some examples, a biomarker is a cluster, e.g., a cluster identified by NMF, e.g., one of the following subtypes: (1) luminal; (2) stromal; (3) immune; and (4) basal. In other examples, a biomarker is a gene. In yet other examples, a biomarker is an alteration (e.g., a somatic alteration). In some aspects, the biomarker is the presence or level of ctDNA in a biological sample obtained from a patient.

The presence and/or expression level/amount of various biomarkers described herein in a sample can be analyzed by any suitable methodologies, including, but not limited to, immunohistochemistry (“IHC”), Western blot analysis, immunoprecipitation, molecular binding assays, ELISA, ELIFA, flow cytometry, fluorescence activated cell sorting (“FACS”), MASSARRAY®, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, massively parallel DNA sequencing (e.g., next-generation sequencing), NANOSTRING®, polymerase chain reaction (PCR), including quantitative real time PCR (qRT-PCR) and reverse transcription-quantitative polymerase chain reaction (RT-qPCR), and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

As used herein, “circulating tumor DNA” and “ctDNA” refer to tumor-derived DNA in the circulatory system that is not associated with cells. ctDNA is a type of cell-free DNA (cfDNA) that may originate from tumor cells or from circulating tumor cells (CTCs). ctDNA may be found, e.g., in the bloodstream of a patient, or in a biological sample (e.g., blood, serum, plasma, or urine) obtained from a patient. In some embodiments, ctDNA may include aberrant mutations (e.g., patient-specific variants) and/or methylation patterns.

The “amount” or “level” of a biomarker associated with an increased clinical benefit to an individual is a detectable level in a biological sample. These can be measured by methods known to one skilled in the art and are also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “diagnosis” is used herein to refer to the identification or classification of a molecular or pathological state, disease or condition (e.g., cancer (e.g., bladder cancer (e.g., UC, e.g., a locally advanced or metastatic UC))). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)). In some examples, a patient may be diagnosed by classifying the patient's cancer according to the methods disclosed herein, e.g., by assigning the patient's tumor sample into one of the following four subtypes based on the transcriptional profile of the patient's tumor: (1) luminal; (2) stromal; (3) immune; and (4) basal.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., a bladder cancer (e.g., UC) tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+T lymphocytes and/or CD4+T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” “control tissue,” or “reference level,” as used herein, refers to a sample, cell, tissue, standard, or level that is used for comparison purposes. In one embodiment, a reference sample, reference cell, reference tissue, control sample, control cell, control tissue, or reference level is obtained from a healthy and/or non-diseased part of the body (e.g., tissue or cells) of the same patient. For example, the reference sample, reference cell, reference tissue, control sample, control cell, control tissue, or reference level may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same patient. In yet another embodiment, a reference sample, reference cell, reference tissue, control sample, control cell, control tissue, or reference level is obtained from a healthy and/or non-diseased part of the body (e.g., tissues or cells) of an individual who is not the patient. In even another embodiment, a reference sample, reference cell, reference tissue, control sample, control cell, control tissue, or reference level is obtained from an untreated tissue and/or cell of the body of an individual who is not the patient. In a further embodiment, a reference level may be obtained from a population of individuals (e.g., a population of patients having a disorder such as cancer (e.g., a bladder cancer such as UC (e.g., locally advanced or metastatic UC)), including a population of patients that does not include the patient being assessed or treated according to a method disclosed herein.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

The phrase “based on” when used herein means that the information about one or more biomarkers is used to inform a treatment decision, information provided on a package insert, or marketing/promotional guidance, and the like. For example, a patient may be selected for an anti-cancer therapy and/or treated with an anti-cancer therapy based on classification of the patient as disclosed herein, e.g., by assignment of the patient's tumor sample into one of the following four subtypes based on the transcriptional profile of the patient's tumor: (1) luminal; (2) stromal; (3) immune; and (4) basal. In another example, a patient may be selected for an anti-cancer therapy and/or treated with an anti-cancer therapy based on the presence of a somatic alteration in the patient's genotype in one or more of the following genes: FGFR3, CDKN2A, and/or CDK2NB.

As used herein, the terms “mutational load,” “mutation load,” “mutational burden,” “tumor mutational burden score,” “TMB score,” “tissue tumor mutational burden score,” and “tTMB score” each of which may be used interchangeably, refer to the level (e.g., number) of an alteration (e.g., one or more alterations, e.g., one or more somatic alterations) per a pre-selected unit (e.g., per megabase) in a pre-determined set of genes (e.g., in the coding regions of the pre-determined set of genes) detected in a tumor tissue sample (e.g., a formalin-fixed and paraffin-embedded (FFPE) tumor sample, an archival tumor sample, a fresh tumor sample, or a frozen tumor sample). The tTMB score can be measured, for example, on a whole genome or exome basis, or on the basis of a subset of the genome or exome. In certain embodiments, the tTMB score measured on the basis of a subset of the genome or exome can be extrapolated to determine a whole genome or exome mutation load. In some embodiments, a tTMB score refers to the level of accumulated somatic mutations within a patient. The tTMB score may refer to accumulated somatic mutations in a patient with cancer (e.g., UC). In some embodiments, a tTMB score refers to the accumulated mutations in the whole genome of a patient. In some embodiments, a tTMB score refers to the accumulated mutations within a particular tissue sample (e.g., tumor tissue sample biopsy, e.g., a urothelial carcinoma tumor sample) collected from a patient. For example, in some embodiments, mutation load may be assessed as described in any one the following publications: U.S. Pat. No. 11,279,767; and U.S. Patent Application Publication Nos. US 2018/0363066, US 2019/0025308, and US 2019/0219586.

The terms “somatic variant,” “somatic mutation,” or “somatic alteration” refer to a genetic alteration occurring in the somatic tissues (e.g., cells outside the germline). Examples of genetic alterations include, but are not limited to, point mutations (e.g., the exchange of a single nucleotide for another (e.g., silent mutations, missense mutations, and nonsense mutations)), insertions and deletions (e.g., the addition and/or removal of one or more nucleotides (e.g., indels)), amplifications, gene duplications, copy number alterations (CNAs), rearrangements, and splice variants. The presence of particular mutations can be associated with disease states (e.g., cancer, e.g., UC).

The term “multiplex-PCR” refers to a single PCR reaction carried out on nucleic acid obtained from a single source (e.g., an individual) using more than one primer set for the purpose of amplifying two or more DNA sequences in a single reaction.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real-time polymerase chain reaction” or “qRT-PCR” or “quantitative PCR” or “qPCR” refers to a form of PCR wherein the amount of PCR product is measured at each step in a PCR reaction. This technique has been described in various publications including, for example, Cronin et al., Am. J. Pathol. 164 (1): 35-42 (2004) and Ma et al., Cancer Cell 5:607-616 (2004).

The term “microarray” refers to an ordered arrangement of hybridizable array elements, preferably polynucleotide probes, on a substrate.

“RNA sequencing” or “RNA-seq,” also called “Whole Transcriptome Shotgun Sequencing (WTSS),” refers to the use of high-throughput sequencing technologies to sequence and/or quantify cDNA to obtain information about a sample's RNA content. Publications describing RNA-seq include: Wang et al. Nature Reviews Genetics 10 (1): 57-63, 2009; Ryan et al. BioTechniques 45 (1): 81-94, 2008; and Maher et al. Nature 458 (7234): 97-101, 2009.

### II. Methods of Classifying Bladder Cancer

Provided herein are methods for classifying bladder cancer (e.g., a UC, e.g., a locally advanced or metastatic UC, including in the 1L, 2L, and later (2L+) treatment settings), which may involve assigning a sample (e.g., a tumor sample) from the patient into a subtype as disclosed herein.

In one example, provided herein is a method of classifying a bladder cancer (e.g., UC, e.g., locally advanced or metastatic UC, including in the 1L, 2L, and later (2L+) treatment settings) in a human patient, the method comprising assigning a patient's tumor sample into one of the following four subtypes based on a transcriptional profile of the patient's tumor: luminal, stromal, immune, or basal, thereby classifying the UC in the patient. In some examples, the transcriptional profile has been provided by assaying mRNA in a sample (e.g., a tumor sample) from the patient.

In another example, provided herein is a method of classifying a bladder cancer (e.g., UC, e.g., locally advanced or metastatic UC, including in the 1L, 2L, and later (2L+) treatment settings) in a human patient, the method comprising: (a) assaying mRNA in a tumor sample from the patient to provide a transcriptional profile of the patient's tumor; and (b) assigning the patient's tumor sample into one of the following four subtypes based on the transcriptional profile of the patient's tumor: luminal, stromal, immune, or basal, thereby classifying the UC in the patient.

In some examples, the patient is previously untreated for the bladder cancer, e.g., UC. In some examples, the patient has received a previous treatment for the bladder cancer, e.g., UC.

Any suitable approach for assaying mRNA may be used. In some examples, assaying mRNA in the tumor sample from the patient comprises RNA sequencing (RNA-seq), reverse transcription-quantitative polymerase chain reaction (RT-qPCR), qPCR, multiplex qPCR or RT-qPCR, microarray analysis, serial analysis of gene expression (SAGE), MASSARRAY® technique, in situ hybridization (ISH), or a combination thereof. In some particular examples, assaying mRNA in the tumor sample from the patient comprises RNA-seq.

Any suitable approach can be used to identify clusters into which a patient's sample (e.g., tumor sample) may be assigned. For example, in some examples, subtypes are identified by non-negative matrix factorization (NMF; see, e.g., Lee et al. Nature 401 (6755): 788-791, 1999 and Brunet et al. Proc. Nat'l Acad. Sci. USA 101:4164-4169, 2004), hierarchical clustering (see, e.g., Eisen et al. Proc. Nat'l Acad. Sci. USA 95 (25): 14863-8, 1998), partition clustering (e.g., K-means clustering, K-medoids clustering, or partitioning around medoids (PAM, see, e.g., Kaufman et al. Finding Groups in Data: John Wiley and Sons, Inc. 2008, pages 68-125)), model-based clustering (e.g., gaussian mixture models), principal component analysis, clustering with deep learning (see, e.g., Li et al. Nat. Commun. 11:2338, 2020), self-organizing map (see, e.g., Kohonen et al. Biol. Cybernet. 43 (1): 59-69, 1982), density-based spatial clustering of applications with noise (DBSCAN, see, e.g., Ester et al. Proceedings of the Second International Conference on Knowledge Discovery and Data Mining; Portland, Oregon: 3001507: AAAI Press; 1996. p. 226-31), and the like. In some examples, hierarchical clustering may include single-linkage, average-linkage, or complete-linkage hierarchical clustering algorithms. Reviews of exemplary clustering approaches are provided, e.g., in Oyalade et al. Bioinform. And Biol. Insights 10:237-253, 2016; Vidman et al. PLoS One 14 (12) e0219102, 2019; and Jamail and Moussa, IntechOpen (DOI: 10.5772/intechopen.94069). In particular examples, subtypes are identified by non-negative NMF, e.g., as described herein in Example 1.

In some examples, RNA-seq count data may be transformed prior to cluster analysis. Any suitable transformation approach can be used, e.g., logarithmic transformation (e.g., log 2-transformation), variance stabilizing transformation, eight data transformation, and the like.

In some examples, the four subtypes are identified by NMF. In some examples, the four subtypes identified by NMF are based on a set of genes representing the top 10% most variable genes in a population of patients having UC (e.g., a locally advanced or metastatic UC, including in the 1L, 2L, and later (2L+) treatment settings).

Any of the methods described herein may include classification of a patient's sample into a subtype, e.g., any subtype identified herein. For example, machine learning algorithms can be used to develop a classifier from gene expression data. Any suitable machine learning algorithm can be used, including supervised learning (e.g., decision tree, random forest, gradient boost machine (GBM), CATBOOST, XGBOOST, support vector machine (SVM), principal component analysis (PCA), K-nearest neighbor, and naïve Bayes) and unsupervised learning approaches. In particular instances, the machine learning algorithm is a random forest algorithm, as described, e.g., in Example 1. For example, a classifier can be developed using the random forest machine learning algorithm (e.g., using the R package randomForest). The random forest classifier can be learned on a training gene set and then used to predict the cluster (e.g., NMF classes) in a second gene set. In other instances, K-means clustering, K-medoids clustering, or PAM can be used for classification.

In some examples, a classifier may be used to assign a patient's tumor to a subtype as disclosed herein. In some examples, a classifier comprising the set of genes set forth in Table 1, or any subset thereof, is used to assign a patient's tumor to a subtype as disclosed herein.

TABLE 1
Genes Representing Top 10% Most Variable Transcripts in Urothelial Carcinoma

| Gene ID | Symbol | Gene Name | Gene ID | Symbol | Gene Name |
|---|---|---|---|---|---|
| 64757 | MTARC1 | mitochondrial amidoxime | 284379 | LOC284379 | solute carrier family 7 member 3 |
| | | reducing component 1 | | | pseudogene |
| 1731 | SEPTIN1 | septin 1 | 647859 | LOC647859 | occludin pseudogene |
| 2 | A2M | alpha-2-macroglobulin | 728392 | LOC728392 | uncharacterized LOC728392 |
| 144568 | A2ML1 | alpha-2-macroglobulin like 1 | 729737 | LOC729737 | uncharacterized LOC729737 |
| 13 | AADAC | arylacetamide deacetylase | 4015 | LOX | lysyl oxidase |
| 18 | ABAT | 4-aminobutyrate | 4016 | LOXL1 | lysyl oxidase like 1 |
| | | aminotransferase | | | |
| 10349 | ABCA10 | ATP binding cassette subfamily | 4017 | LOXL2 | lysyl oxidase like 2 |
| | | A member 10 | | | |
| 26154 | ABCA12 | ATP binding cassette subfamily | 84171 | LOXL4 | lysyl oxidase like 4 |
| | | A member 12 | | | |
| 154664 | ABCA13 | ATP binding cassette subfamily | 4018 | LPA | lipoprotein(a) |
| | | A member 13 | | | |
| 21 | ABCA3 | ATP binding cassette subfamily | 23566 | LPAR3 | lysophosphatidic acid receptor 3 |
| | | A member 3 | | | |
| 23460 | ABCA6 | ATP binding cassette subfamily | 4023 | LPL | lipoprotein lipase |
| | | A member 6 | | | |
| 10351 | ABCA8 | ATP binding cassette subfamily | 57631 | LRCH2 | leucine rich repeats and calponin |
| | | A member 8 | | | homology domain containing 2 |
| 10350 | ABCA9 | ATP binding cassette subfamily | 145581 | LRFN5 | leucine rich repeat and |
| | | A member 9 | | | fibronectin type III domain |
| | | | | | containing 5 |
| 5243 | ABCB1 | ATP binding cassette subfamily | 116844 | LRG1 | leucine rich alpha-2-glycoprotein |
| | | B member 1 | | | 1 |
| 8714 | ABCC3 | ATP binding cassette subfamily | 26018 | LRIG1 | leucine rich repeats and |
| | | C member 3 | | | immunoglobulin like domains 1 |
| 10060 | ABCC9 | ATP binding cassette subfamily | 4033 | LRMP | lymphoid restricted membrane |
| | | C member 9 | | | protein |
| 25890 | ABI3BP | ABI family member 3 binding | 53353 | LRP1B | LDL receptor related protein 1B |
| | | protein | | | |
| 22885 | ABLIM3 | actin binding LIM protein family | 4038 | LRP4 | LDL receptor related protein 4 |
| | | member 3 | | | |
| 28 | ABO | ABO, alpha 1-3-N- | 131578 | LRRC15 | leucine rich repeat containing 15 |
| | | acetylgalactosaminyltransferase | | | |
| | | and alpha 1-3- | | | |
| | | galactosyltransferase | | | |
| 32 | ACACB | acetyl-CoA carboxylase beta | 10234 | LRRC17 | leucine rich repeat containing 17 |
| 33 | ACADL | acyl-CoA dehydrogenase long | 2615 | LRRC32 | leucine rich repeat containing 32 |
| | | chain | | | |
| 176 | ACAN | aggrecan | 55073 | LRRC37A4P | leucine rich repeat containing 37 |
| | | | | | member A4, pseudogene |
| 9744 | ACAP1 | ArfGAP with coiled-coil, ankyrin | 387646 | LRRC37A6P | leucine rich repeat containing 37 |
| | | repeat and PH domains 1 | | | member A6, pseudogene |
| 340485 | ACER2 | alkaline ceramidase 2 | 120892 | LRRK2 | leucine rich repeat kinase 2 |
| 2532 | ACKR1 | atypical chemokine receptor 1 | 57633 | LRRN1 | leucine rich repeat neuronal 1 |
| | | (Duffy blood group) | | | |
| 1238 | ACKR2 | atypical chemokine receptor 2 | 4046 | LSP1 | lymphocyte-specific protein 1 |
| 57007 | ACKR3 | atypical chemokine receptor 3 | 7940 | LST1 | leukocyte specific transcript 1 |
| 51 | ACOX1 | acyl-CoA oxidase 1 | 4050 | LTB | lymphotoxin beta |
| 8309 | ACOX2 | acyl-CoA oxidase 2 | 4053 | LTBP2 | latent transforming growth factor |
| | | | | | beta binding protein 2 |
| 55289 | ACOXL | acyl-CoA oxidase like | 8425 | LTBP4 | latent transforming growth factor |
| | | | | | beta binding protein 4 |
| 51205 | ACP6 | acid phosphatase 6, | 4057 | LTF | lactotransferrin |
| | | lysophosphatidic | | | |
| 55 | ACPP | acid phosphatase, prostate | 4060 | LUM | lumican |
| 80221 | ACSF2 | acyl-CoA synthetase family | 56925 | LXN | latexin |
| | | member 2 | | | |
| 51703 | ACSL5 | acyl-CoA synthetase long chain | 8581 | LY6D | lymphocyte antigen 6 family |
| | | family member 5 | | | member D |
| 6296 | ACSM3 | acyl-CoA synthetase medium | 4061 | LY6E | lymphocyte antigen 6 family |
| | | chain family member 3 | | | member E |
| 142827 | ACSM6 | acyl-CoA synthetase medium | 54742 | LY6K | lymphocyte antigen 6 family |
| | | chain family member 6 | | | member K |
| 79611 | ACSS3 | acyl-CoA synthetase short chain | 9450 | LY86 | lymphocyte antigen 86 |
| | | family member 3 | | | |
| 59 | ACTA2 | actin, alpha 2, smooth muscle, | 4063 | LY9 | lymphocyte antigen 9 |
| | | aorta | | | |
| 70 | ACTC1 | actin, alpha, cardiac muscle 1 | 23643 | LY96 | lymphocyte antigen 96 |
| 72 | ACTG2 | actin, gamma 2, smooth muscle, | 27076 | LYPD3 | LY6/PLAUR domain containing 3 |
| | | enteric | | | |
| 87 | ACTN1 | actinin alpha 1 | 130574 | LYPD6 | LY6/PLAUR domain containing 6 |
| 88 | ACTN2 | actinin alpha 2 | 130576 | LYPD6B | LY6/PLAUR domain containing |
| | | | | | 6B |
| 8038 | ADAM12 | ADAM metallopeptidase domain | 10894 | LYVE1 | lymphatic vessel endothelial |
| | | 12 | | | hyaluronan receptor 1 |
| 8728 | ADAM19 | ADAM metallopeptidase domain | 4069 | LYZ | lysozyme |
| | | 19 | | | |
| 53616 | ADAM22 | ADAM metallopeptidase domain | 89876 | MAATS1 | MYCBP associated and testis |
| | | 22 | | | expressed 1 |
| 10863 | ADAM28 | ADAM metallopeptidase domain | 126868 | MAB21L3 | mab-21 like 3 |
| | | 28 | | | |
| 80332 | ADAM33 | ADAM metallopeptidase domain | 346389 | MACC1 | MACC1, MET transcriptional |
| | | 33 | | | regulator |
| 27299 | ADAMDEC1 | ADAM like decysin 1 | 140733 | MACROD2 | MACRO domain containing 2 |
| 9510 | ADAMTS1 | ADAM metallopeptidase with | 4102 | MAGEA3 | MAGE family member A3 |
| | | thrombospondin type 1 motif 1 | | | |
| 81794 | ADAMTS10 | ADAM metallopeptidase with | 4105 | MAGEA6 | MAGE family member A6 |
| | | thrombospondin type 1 motif 10 | | | |
| 81792 | ADAMTS12 | ADAM metallopeptidase with | 9863 | MAGI2 | membrane associated guanylate |
| | | thrombospondin type 1 motif 12 | | | kinase, WW and PDZ domain |
| | | | | | containing 2 |
| 140766 | ADAMTS14 | ADAM metallopeptidase with | 100506898 | MAGOH2P | mago homolog 2, pseudogene |
| | | thrombospondin type 1 motif 14 | | | |
| 170690 | ADAMTS16 | ADAM metallopeptidase with | 4118 | MAL | mal, T-cell differentiation protein |
| | | thrombospondin type 1 motif 16 | | | |
| 9509 | ADAMTS2 | ADAM metallopeptidase with | 114569 | MAL2 | mal, T-cell differentiation protein |
| | | thrombospondin type 1 motif 2 | | | 2 (gene/pseudogene) |
| 80070 | ADAMTS20 | ADAM metallopeptidase with | 7851 | MALL | mal, T-cell differentiation protein |
| | | thrombospondin type 1 motif 20 | | | like |
| 9507 | ADAMTS4 | ADAM metallopeptidase with | 256691 | MAMDC2 | MAM domain containing 2 |
| | | thrombospondin type 1 motif 4 | | | |
| 11174 | ADAMTS6 | ADAM metallopeptidase with | 57134 | MAN1C1 | mannosidase alpha class 1C |
| | | thrombospondin type 1 motif 6 | | | member 1 |
| 11095 | ADAMTS8 | ADAM metallopeptidase with | 149175 | MANEAL | mannosidase endo-alpha like |
| | | thrombospondin type 1 motif 8 | | | |
| 56999 | ADAMTS9 | ADAM metallopeptidase with | 4128 | MAOA | monoamine oxidase A |
| | | thrombospondin type 1 motif 9 | | | |
| 92949 | ADAMTSL1 | ADAMTS like 1 | 4129 | MAOB | monoamine oxidase B |
| 57188 | ADAMTSL3 | ADAMTS like 3 | 4130 | MAP1A | microtubule associated protein |
| | | | | | 1A |
| 54507 | ADAMTSL4 | ADAMTS like 4 | 4131 | MAP1B | microtubule associated protein |
| | | | | | 1B |
| 108 | ADCY2 | adenylate cyclase 2 | 11184 | MAP4K1 | mitogen-activated protein kinase |
| | | | | | kinase kinase kinase 1 |
| 111 | ADCY5 | adenylate cyclase 5 | 5602 | MAPK10 | mitogen-activated protein kinase |
| | | | | | 10 |
| 25960 | ADGRA2 | adhesion G protein-coupled | 225689 | MAPK15 | mitogen-activated protein kinase |
| | | receptor A2 | | | 15 |
| 577 | ADGRB3 | adhesion G protein-coupled | 8685 | MARCO | macrophage receptor with |
| | | receptor B3 | | | collagenous structure |
| 283383 | ADGRD1 | adhesion G protein-coupled | 5648 | MASP1 | mannan binding lectin serine |
| | | receptor D1 | | | peptidase 1 |
| 84658 | ADGRE3 | adhesion G protein-coupled | 4147 | MATN2 | matrilin 2 |
| | | receptor E3 | | | |
| 266977 | ADGRF1 | adhesion G protein-coupled | 4148 | MATN3 | matrilin 3 |
| | | receptor F1 | | | |
| 221393 | ADGRF4 | adhesion G protein-coupled | 55388 | MCM10 | minichromosome maintenance |
| | | receptor F4 | | | 10 replication initiation factor |
| 9289 | ADGRG1 | adhesion G protein-coupled | 4171 | MCM2 | minichromosome maintenance |
| | | receptor G1 | | | complex component 2 |
| 57211 | ADGRG6 | adhesion G protein-coupled | 255231 | MCOLN2 | mucolipin 2 |
| | | receptor G6 | | | |
| 23284 | ADGRL3 | adhesion G protein-coupled | 55283 | MCOLN3 | mucolipin 3 |
| | | receptor L3 | | | |
| 84059 | ADGRV1 | adhesion G protein-coupled | 4188 | MDFI | MyoD family inhibitor |
| | | receptor V1 | | | |
| 125 | ADH1B | alcohol dehydrogenase 1B (class | 4192 | MDK | midkine |
| | | I), beta polypeptide | | | |
| 126 | ADH1C | alcohol dehydrogenase 1C (class | 2122 | MECOM | MDS1 and EVI1 complex locus |
| | | I), gamma polypeptide | | | |
| 137872 | ADHFE1 | alcohol dehydrogenase, iron | 84935 | MEDAG | mesenteric estrogen dependent |
| | | containing 1 | | | adipogenesis |
| 10974 | ADIRF | adipogenesis regulatory factor | 4210 | MEFV | MEFV, pyrin innate immunity |
| | | | | | regulator |
| 133 | ADM | adrenomedullin | 4211 | MEIS1 | Meis homeobox 1 |
| 136 | ADORA2B | adenosine A2b receptor | 4212 | MEIS2 | Meis homeobox 2 |
| 154 | ADRB2 | adrenoceptor beta 2 | 9833 | MELK | maternal embryonic leucine |
| | | | | | zipper kinase |
| 122622 | ADSSL1 | adenylosuccinate synthase like 1 | 4223 | MEOX2 | mesenchyme homeobox 2 |
| 84830 | ADTRP | androgen dependent TFPI | 4232 | MEST | mesoderm specific transcript |
| | | regulating protein | | | |
| 165 | AEBP1 | AE binding protein 1 | 4233 | MET | MET proto-oncogene, receptor |
| | | | | | tyrosine kinase |
| 84632 | AFAP1L2 | actin filament associated protein | 728464 | METTL24 | methyltransferase like 24 |
| | | 1 like 2 | | | |
| 3899 | AFF3 | AF4/FMR2 family member 3 | 155368 | METTL27 | methyltransferase like 27 |
| 119385 | AGAP11 | ArfGAP with GTPase domain, | 25840 | METTL7A | methyltransferase like 7A |
| | | ankyrin repeat and PH domain | | | |
| | | 11 | | | |
| 414224 | AGAP12P | ArfGAP with GTPase domain, | 4237 | MFAP2 | microfibril associated protein 2 |
| | | ankyrin repeat and PH domain | | | |
| | | 12, pseudogene | | | |
| 392636 | AGMO | alkylglycerol monooxygenase | 9848 | MFAP3L | microfibril associated protein 3 |
| | | | | | like |
| 10551 | AGR2 | anterior gradient 2, protein | 4239 | MFAP4 | microfibril associated protein 4 |
| | | disulphide isomerase family | | | |
| | | member | | | |
| 155465 | AGR3 | anterior gradient 3, protein | 8076 | MFAP5 | microfibril associated protein 5 |
| | | disulphide isomerase family | | | |
| | | member | | | |
| 183 | AGT | angiotensinogen | 84879 | MFSD2A | major facilitator superfamily |
| | | | | | domain containing 2A |
| 185 | AGTR1 | angiotensin II receptor type 1 | 8972 | MGAM | maltase-glucoamylase |
| 113146 | AHNAK2 | AHNAK nucleoprotein 2 | 4256 | MGP | matrix Gla protein |
| 199 | AIF1 | allograft inflammatory factor 1 | 4257 | MGST1 | microsomal glutathione S- |
| | | | | | transferase 1 |
| 83543 | AIF1L | allograft inflammatory factor 1 | 84953 | MICALCL | MICAL C-terminal like |
| | | like | | | |
| 150209 | AIFM3 | apoptosis inducing factor, | 4277 | MICB | MHC class I polypeptide-related |
| | | mitochondria associated 3 | | | sequence B |
| 9447 | AIM2 | absent in melanoma 2 | 642587 | MIR205HG | MIR205 host gene |
| 9590 | AKAP12 | A-kinase anchoring protein 12 | 100313769 | MIR320B2 | microRNA 320b-2 |
| 9472 | AKAP6 | A-kinase anchoring protein 6 | 100506755 | MIR497HG | mir-497-195 cluster host gene |
| 231 | AKR1B1 | aldo-keto reductase family 1 | 693197 | MIR612 | microRNA 612 |
| | | member B | | | |
| 57016 | AKR1B10 | aldo-keto reductase family 1 | 723778 | MIR650 | microRNA 650 |
| | | member B10 | | | |
| 1645 | AKR1C1 | aldo-keto reductase family 1 | 126353 | MISP | mitotic spindle positioning |
| | | member C1 | | | |
| 1646 | AKR1C2 | aldo-keto reductase family 1 | 4286 | MITF | melanogenesis associated |
| | | member C2 | | | transcription factor |
| 8644 | AKR1C3 | aldo-keto reductase family 1 | 4288 | MKI67 | marker of proliferation Ki-67 |
| | | member C3 | | | |
| 83592 | AKR1E2 | aldo-keto reductase family 1 | 100129480 | MKRN2OS | MKRN2 opposite strand |
| | | member E2 | | | |
| 10000 | AKT3 | AKT serine/threonine kinase 3 | 79083 | MLPH | melanophilin |
| 214 | ALCAM | activated leukocyte cell adhesion | 4311 | MME | membrane metalloendopeptidase |
| | | molecule | | | |
| 216 | ALDH1A1 | aldehyde dehydrogenase 1 | 4312 | MMP1 | matrix metallopeptidase 1 |
| | | family member A1 | | | |
| 8854 | ALDH1A2 | aldehyde dehydrogenase 1 | 4319 | MMP10 | matrix metallopeptidase 10 |
| | | family member A2 | | | |
| 220 | ALDH1A3 | aldehyde dehydrogenase 1 | 4320 | MMP11 | matrix metallopeptidase 11 |
| | | family member A3 | | | |
| 219 | ALDH1B1 | aldehyde dehydrogenase 1 | 4321 | MMP12 | matrix metallopeptidase 12 |
| | | family member B1 | | | |
| 10840 | ALDH1L1 | aldehyde dehydrogenase 1 | 4322 | MMP13 | matrix metallopeptidase 13 |
| | | family member L1 | | | |
| 160428 | ALDH1L2 | aldehyde dehydrogenase 1 | 4323 | MMP14 | matrix metallopeptidase 14 |
| | | family member L2 | | | |
| 217 | ALDH2 | aldehyde dehydrogenase 2 | 4327 | MMP19 | matrix metallopeptidase 19 |
| | | family (mitochondrial) | | | |
| 218 | ALDH3A1 | aldehyde dehydrogenase 3 | 4313 | MMP2 | matrix metallopeptidase 2 |
| | | family member A1 | | | |
| 222 | ALDH3B2 | aldehyde dehydrogenase 3 | 79148 | MMP28 | matrix metallopeptidase 28 |
| | | family member B2 | | | |
| 8659 | ALDH4A1 | aldehyde dehydrogenase 4 | 4314 | MMP3 | matrix metallopeptidase 3 |
| | | family member A1 | | | |
| 501 | ALDH7A1 | aldehyde dehydrogenase 7 | 4316 | MMP7 | matrix metallopeptidase 7 |
| | | family member A1 | | | |
| 200810 | ALG1L | ALG1, | 4318 | MMP9 | matrix metallopeptidase 9 |
| | | chitobiosyldiphosphodolichol | | | |
| | | beta-mannosyltransferase like | | | |
| 389658 | ALKAL1 | ALK and LTK ligand 1 | 22915 | MMRN1 | multimerin 1 |
| 239 | ALOX12 | arachidonate 12-lipoxygenase, | 79812 | MMRN2 | multimerin 2 |
| | | 12S type | | | |
| 246 | ALOX15 | arachidonate 15-lipoxygenase | 4332 | MNDA | myeloid cell nuclear |
| | | | | | differentiation antigen |
| 240 | ALOX5 | arachidonate 5-lipoxygenase | 26002 | MOXD1 | monooxygenase DBH like 1 |
| 241 | ALOX5AP | arachidonate 5-lipoxygenase | 219972 | MPEG1 | macrophage expressed 1 |
| | | activating protein | | | |
| 115701 | ALPK2 | alpha kinase 2 | 744 | MPPED2 | metallophosphoesterase domain |
| | | | | | containing 2 |
| 259173 | ALS2CL | ALS2 C-terminal like | 10205 | MPZL2 | myelin protein zero like 2 |
| 347902 | AMIGO2 | adhesion molecule with lg like | 4360 | MRC1 | mannose receptor C-type 1 |
| | | domain 2 | | | |
| 154796 | AMOT | angiomotin | 9902 | MRC2 | mannose receptor C type 2 |
| 270 | AMPD1 | adenosine monophosphate | 116535 | MRGPRF | MAS related GPR family member |
| | | deaminase 1 | | | F |
| 280 | AMY2B | amylase, alpha 2B (pancreatic) | 10335 | MRVI1 | murine retrovirus integration site |
| | | | | | 1 homolog |
| 9068 | ANGPTL1 | angiopoietin like 1 | 931 | MS4A1 | membrane spanning 4-domains |
| | | | | | A1 |
| 23452 | ANGPTL2 | angiopoietin like 2 | 84689 | MS4A14 | membrane spanning 4-domains |
| | | | | | A14 |
| 51129 | ANGPTL4 | angiopoietin like 4 | 2206 | MS4A2 | membrane spanning 4-domains |
| | | | | | A2 |
| 287 | ANK2 | ankyrin 2 | 51338 | MS4A4A | membrane spanning 4-domains |
| | | | | | A4A |
| 162282 | ANKFN1 | ankyrin repeat and fibronectin | 64231 | MS4A6A | membrane spanning 4-domains |
| | | type III domain containing 1 | | | A6A |
| 27063 | ANKRD1 | ankyrin repeat domain 1 | 9242 | MSC | musculin |
| 253650 | ANKRD18A | ankyrin repeat domain 18A | 4477 | MSMB | microseminoprotein beta |
| 441459 | ANKRD18B | ankyrin repeat domain 18B | 4481 | MSR1 | macrophage scavenger receptor |
| | | | | | 1 |
| 138649 | ANKRD19P | ankyrin repeat domain 19, | 253827 | MSRB3 | methionine sulfoxide reductase |
| | | pseudogene | | | B3 |
| 391267 | ANKRD20A11P | ankyrin repeat domain 20 family | 4486 | MST1R | macrophage stimulating 1 |
| | | member A11, pseudogene | | | receptor |
| 440482 | ANKRD20A5P | ankyrin repeat domain 20 family | 4488 | MSX2 | msh homeobox 2 |
| | | member A5, pseudogene | | | |
| 118932 | ANKRD22 | ankyrin repeat domain 22 | 4489 | MT1A | metallothionein 1A |
| 147463 | ANKRD29 | ankyrin repeat domain 29 | 4493 | MT1E | metallothionein 1E |
| 645784 | ANKRD36BP2 | ankyrin repeat domain 36B | 4494 | MT1F | metallothionein 1F |
| | | pseudogene 2 | | | |
| 57182 | ANKRD50 | ankyrin repeat domain 50 | 4495 | MT1G | metallothionein 1G |
| 441869 | ANKRD65 | ankyrin repeat domain 65 | 4496 | MT1H | metallothionein 1H |
| 56899 | ANKS1B | ankyrin repeat and sterile alpha | 4500 | MT1L | metallothionein 1L, pseudogene |
| | | motif domain containing 1B | | | |
| 54443 | ANLN | anillin actin binding protein | 4499 | MT1M | metallothionein 1M |
| 55107 | ANO1 | anoctamin 1 | 4501 | MT1X | metallothionein 1X |
| 203859 | ANO5 | anoctamin 5 | 4502 | MT2A | metallothionein 2A |
| 3730 | ANOS1 | anosmin 1 | 4507 | MTAP | methylthioadenosine |
| | | | | | phosphorylase |
| 23520 | ANP32C | acidic nuclear phosphoprotein 32 | 23255 | MTCL1 | microtubule crosslinking factor 1 |
| | | family member C | | | |
| 290 | ANPEP | alanyl aminopeptidase, | 4582 | MUC1 | mucin 1, cell surface associated |
| | | membrane | | | |
| 84168 | ANTXR1 | anthrax toxin receptor 1 | 143662 | MUC15 | mucin 15, cell surface associated |
| 118429 | ANTXR2 | anthrax toxin receptor 2 | 94025 | MUC16 | mucin 16, cell surface associated |
| 301 | ANXA1 | annexin A1 | 200958 | MUC20 | mucin 20, cell surface associated |
| 11199 | ANXA10 | annexin A10 | 4584 | MUC3A | mucin 3A, cell surface associated |
| 306 | ANXA3 | annexin A3 | 4585 | MUC4 | mucin 4, cell surface associated |
| 309 | ANXA6 | annexin A6 | 118430 | MUCL1 | mucin like 1 |
| 653145 | ANXA8 | annexin A8 | 4599 | MX1 | MX dynamin like GTPase 1 |
| 728113 | ANXA8L1 | annexin A8 like 1 | 25878 | MXRA5 | matrix remodeling associated 5 |
| 8416 | ANXA9 | annexin A9 | 54587 | MXRA8 | matrix remodeling associated 8 |
| 313 | AOAH | acyloxyacyl hydrolase | 91663 | MYADM | myeloid associated differentiation |
| | | | | | marker |
| 8639 | AOC3 | amine oxidase, copper | 4602 | MYB | MYB proto-oncogene, |
| | | containing 3 | | | transcription factor |
| 316 | AOX1 | aldehyde oxidase 1 | 4605 | MYBL2 | MYB proto-oncogene like 2 |
| 321 | APBA2 | amyloid beta precursor protein | 4604 | MYBPC1 | myosin binding protein C, slow |
| | | binding family A member 2 | | | type |
| 54518 | APBB1IP | amyloid beta precursor protein | 4609 | MYC | MYC proto-oncogene, bHLH |
| | | binding family B member 1 | | | transcription factor |
| | | interacting protein | | | |
| 147495 | APCDD1 | APC down-regulated 1 | 4610 | MYCL | MYCL proto-oncogene, bHLH |
| | | | | | transcription factor |
| 9582 | APOBEC3B | apolipoprotein B mRNA editing | 4613 | MYCN | MYCN proto-oncogene, bHLH |
| | | enzyme catalytic subunit 3B | | | transcription factor |
| 60489 | APOBEC3G | apolipoprotein B mRNA editing | 50804 | MYEF2 | myelin expression factor 2 |
| | | enzyme catalytic subunit 3G | | | |
| 341 | APOC1 | apolipoprotein C1 | 26579 | MYEOV | myeloma overexpressed |
| 347 | APOD | apolipoprotein D | 4629 | MYH11 | myosin heavy chain 11 |
| 348 | APOE | apolipoprotein E | 79784 | MYH14 | myosin heavy chain 14 |
| 8542 | APOL1 | apolipoprotein L1 | 4621 | MYH3 | myosin heavy chain 3 |
| 80833 | APOL3 | apolipoprotein L3 | 10398 | MYL9 | myosin light chain 9 |
| 80832 | APOL4 | apolipoprotein L4 | 4638 | MYLK | myosin light chain kinase |
| 358 | AQP1 | aquaporin 1 (Colton blood group) | 4542 | MYO1F | myosin IF |
| 360 | AQP3 | aquaporin 3 (Gill blood group) | 64005 | MYO1G | myosin IG |
| 364 | AQP7 | aquaporin 7 | 140469 | MYO3B | myosin IIIB |
| 366 | AQP9 | aquaporin 9 | 4645 | MYO5B | myosin VB |
| 367 | AR | androgen receptor | 4647 | MYO7A | myosin VIIA |
| 374 | AREG | amphiregulin | 93649 | MYOCD | myocardin |
| 57221 | ARFGEF3 | ARFGEF family member 3 | 8736 | MYOM1 | myomesin 1 |
| 384 | ARG2 | arginase 2 | 9499 | MYOT | myotilin |
| 55843 | ARHGAP15 | Rho GTPase activating protein | 51237 | MZB1 | marginal zone B and B1 cell |
| | | 15 | | | specific protein |
| 57636 | ARHGAP23 | Rho GTPase activating protein | 100293211 | NA | NA |
| | | 23 | | | |
| 83478 | ARHGAP24 | Rho GTPase activating protein | 100509457 | NA | NA |
| | | 24 | | | |
| 79822 | ARHGAP28 | Rho GTPase activating protein | 100996760 | NA | NA |
| | | 28 | | | |
| 9411 | ARHGAP29 | Rho GTPase activating protein | 100996809 | NA | NA |
| | | 29 | | | |
| 393 | ARHGAP4 | Rho GTPase activating protein 4 | 101060789 | NA | NA |
| 343578 | ARHGAP40 | Rho GTPase activating protein | 101060846 | NA | NA |
| | | 40 | | | |
| 395 | ARHGAP6 | Rho GTPase activating protein 6 | 101927371 | NA | NA |
| 64333 | ARHGAP9 | Rho GTPase activating protein 9 | 101927733 | NA | NA |
| 27237 | ARHGEF16 | Rho guanine nucleotide | 101929206 | NA | NA |
| | | exchange factor 16 | | | |
| 115557 | ARHGEF25 | Rho guanine nucleotide | 101929585 | NA | NA |
| | | exchange factor 25 | | | |
| 26084 | ARHGEF26 | Rho guanine nucleotide | 101930400 | NA | NA |
| | | exchange factor 26 | | | |
| 64283 | ARHGEF28 | Rho guanine nucleotide | 101930662 | NA | NA |
| | | exchange factor 28 | | | |
| 50649 | ARHGEF4 | Rho guanine nucleotide | 102723891 | NA | NA |
| | | exchange factor 4 | | | |
| 10865 | ARID5A | AT-rich interaction domain 5A | 102724424 | NA | NA |
| 80117 | ARL14 | ADP ribosylation factor like | 102724436 | NA | NA |
| | | GTPase 14 | | | |
| 379 | ARL4D | ADP ribosylation factor like | 102725001 | NA | NA |
| | | GTPase 4D | | | |
| 9915 | ARNT2 | aryl hydrocarbon receptor | 102725018 | NA | NA |
| | | nuclear translocator 2 | | | |
| 56938 | ARNTL2 | aryl hydrocarbon receptor | 170063 | NA | NA |
| | | nuclear translocator like 2 | | | |
| 645432 | ARRDC5 | arrestin domain containing 5 | 645090 | NA | NA |
| 415 | ARSE | arylsulfatase E | 80761 | NA | NA |
| | | (chondrodysplasia punctata 1) | | | |
| 340075 | ARSI | arylsulfatase family member I | 10003 | NAALAD2 | N-acetylated alpha-linked acidic |
| | | | | | dipeptidase 2 |
| 51676 | ASB2 | ankyrin repeat and SOCS box | 254827 | NAALADL2 | N-acetylated alpha-linked acidic |
| | | containing 2 | | | dipeptidase like 2 |
| 443 | ASPA | aspartoacylase | 259232 | NALCN | sodium leak channel, non- |
| | | | | | selective |
| 259266 | ASPM | abnormal spindle microtubule | 4675 | NAP1L3 | nucleosome assembly protein 1 |
| | | assembly | | | like 3 |
| 54829 | ASPN | asporin | 256236 | NAPSB | napsin B aspartic peptidase, |
| | | | | | pseudogene |
| 80150 | ASRGL1 | asparaginase like 1 | 89795 | NAV3 | neuron navigator 3 |
| 445 | ASS1 | argininosuccinate synthase 1 | 26960 | NBEA | neurobeachin |
| 467 | ATF3 | activating transcription factor 3 | 4684 | NCAM1 | neural cell adhesion molecule 1 |
| 80063 | ATF7IP2 | activating transcription factor 7 | 4685 | NCAM2 | neural cell adhesion molecule 2 |
| | | interacting protein 2 | | | |
| 23120 | ATP10B | ATPase phospholipid | 64151 | NCAPG | non-SMC condensin I complex |
| | | transporting 10B (putative) | | | subunit G |
| 477 | ATP1A2 | ATPase Na+/K+ transporting | 342897 | NCCRP1 | non-specific cytotoxic cell |
| | | subunit alpha 2 | | | receptor protein 1 homolog |
| | | | | | (zebrafish) |
| 481 | ATP1B1 | ATPase Na+/K+ transporting | 653361 | NCF1 | neutrophil cytosolic factor 1 |
| | | subunit beta 1 | | | |
| 482 | ATP1B2 | ATPase Na+/K+ transporting | 654817 | NCF1C | neutrophil cytosolic factor 1C |
| | | subunit beta 2 | | | pseudogene |
| 489 | ATP2A3 | ATPase | 4688 | NCF2 | neutrophil cytosolic factor 2 |
| | | sarcoplasmic/endoplasmic | | | |
| | | reticulum Ca2+ transporting 3 | | | |
| 9914 | ATP2C2 | ATPase secretory pathway Ca2+ | 3071 | NCKAP1L | NCK associated protein 1 like |
| | | transporting 2 | | | |
| 4508 | ATP6 | ATP synthase F0 subunit 6 | 4535 | ND1 | NADH dehydrogenase, subunit 1 |
| | | | | | (complex I) |
| 50617 | ATP6V0A4 | ATPase H+ transporting V0 | 4536 | ND2 | MTND2 |
| | | subunit a4 | | | |
| 525 | ATP6V1B1 | ATPase H+ transporting V1 | 4537 | ND3 | NADH dehydrogenase, subunit 3 |
| | | subunit B1 | | | (complex I) |
| 4509 | ATP8 | ATP synthase FO subunit 8 | 4538 | ND4 | NADH dehydrogenase, subunit 4 |
| | | | | | (complex I) |
| 10396 | ATP8A1 | ATPase phospholipid | 4539 | ND4L | NADH dehydrogenase, subunit |
| | | transporting 8A1 | | | 4L (complex I) |
| 5205 | ATP8B1 | ATPase phospholipid | 4540 | ND5 | NADH dehydrogenase, subunit 5 |
| | | transporting 8B1 | | | (complex I) |
| 26033 | ATRNL1 | attractin like 1 | 10403 | NDC80 | NDC80, kinetochore complex |
| | | | | | component |
| 79000 | AUNIP | aurora kinase A and ninein | 79625 | NDNF | neuron derived neurotrophic |
| | | interacting protein | | | factor |
| 6790 | AURKA | aurora kinase A | 10397 | NDRG1 | N-myc downstream regulated 1 |
| 9212 | AURKB | aurora kinase B | 57447 | NDRG2 | NDRG family member 2 |
| 8313 | AXIN2 | axin 2 | 283131 | NEAT1 | nuclear paraspeckle assembly |
| | | | | | transcript 1 (non-protein coding) |
| 563 | AZGP1 | alpha-2-glycoprotein 1, zinc- | 4703 | NEB | nebulin |
| | | binding | | | |
| 10331 | B3GNT3 | UDP-GlcNAc:betaGal beta-1,3- | 10529 | NEBL | nebulette |
| | | N-acetylglucosaminyltransferase | | | |
| | | 3 | | | |
| 84002 | B3GNT5 | UDP-GlcNAc:betaGal beta-1,3- | 5818 | NECTIN1 | nectin cell adhesion molecule 1 |
| | | N-acetylglucosaminyltransferase | | | |
| | | 5 | | | |
| 283358 | B4GALNT3 | beta-1,4-N-acetyl- | 81607 | NECTIN4 | nectin cell adhesion molecule 4 |
| | | galactosaminyltransferase 3 | | | |
| 338707 | B4GALNT4 | beta-1,4-N-acetyl- | 23327 | NEDD4L | neural precursor cell expressed, |
| | | galactosaminyltransferase 4 | | | developmentally down-regulated |
| | | | | | 4-like, E3 ubiquitin protein ligase |
| 9532 | BAG2 | BCL2 associated athanogene 2 | 4744 | NEFH | neurofilament heavy |
| 8938 | BAIAP3 | BAI1 associated protein 3 | 4741 | NEFM | neurofilament medium |
| 25805 | BAMBI | BMP and activin membrane | 257194 | NEGR1 | neuronal growth regulator 1 |
| | | bound inhibitor | | | |
| 55024 | BANK1 | B-cell scaffold protein with | 55247 | NEIL3 | nei like DNA glycosylase 3 |
| | | ankyrin repeats 1 | | | |
| 8538 | BARX2 | BARX homeobox 2 | 4751 | NEK2 | NIMA related kinase 2 |
| 10409 | BASP1 | brain abundant membrane | 4753 | NELL2 | neural EGFL like 2 |
| | | attached signal protein 1 | | | |
| 4059 | BCAM | basal cell adhesion molecule | 81831 | NETO2 | neuropilin and tolloid like 2 |
| | | (Lutheran blood group) | | | |
| 8537 | BCAS1 | breast carcinoma amplified | 91624 | NEXN | nexilin F-actin binding protein |
| | | sequence 1 | | | |
| 586 | BCAT1 | branched chain amino acid | 23114 | NFASC | neurofascin |
| | | transaminase 1 | | | |
| 587 | BCAT2 | branched chain amino acid | 4773 | NFATC2 | nuclear factor of activated T-cells |
| | | transaminase 2 | | | 2 |
| 590 | BCHE | butyrylcholinesterase | 9603 | NFE2L3 | nuclear factor, erythroid 2 like 3 |
| 597 | BCL2A1 | BCL2 related protein A1 | 4781 | NFIB | nuclear factor I B |
| 53630 | BCO1 | beta-carotene oxygenase 1 | 4784 | NFIX | nuclear factor I X |
| 623 | BDKRB1 | bradykinin receptor B1 | 4803 | NGF | nerve growth factor |
| 222389 | BEND7 | BEN domain containing 7 | 4804 | NGFR | nerve growth factor receptor |
| 84707 | BEX2 | brain expressed X-linked 2 | 340527 | NHSL2 | NHS like 2 |
| 340542 | BEX5 | brain expressed X-linked 5 | 4811 | NID1 | nidogen 1 |
| 633 | BGN | biglycan | 22795 | NID2 | nidogen 2 |
| 168620 | BHLHA15 | basic helix-loop-helix family | 22981 | NINL | ninein like |
| | | member a15 | | | |
| 8553 | BHLHE40 | basic helix-loop-helix family | 152519 | NIPAL1 | NIPA like domain containing 1 |
| | | member e40 | | | |
| 79365 | BHLHE41 | basic helix-loop-helix family | 348938 | NIPAL4 | NIPA like domain containing 4 |
| | | member e41 | | | |
| 635 | BHMT | betaine--homocysteine S- | 85409 | NKD2 | naked cuticle homolog 2 |
| | | methyltransferase | | | |
| 23743 | BHMT2 | betaine--homocysteine S- | 4818 | NKG7 | natural killer cell granule protein |
| | | methyltransferase 2 | | | 7 |
| 80114 | BICC1 | BicC family RNA binding protein | 57502 | NLGN4X | neuroligin 4, X-linked |
| | | 1 | | | |
| 146439 | BICDL2 | BICD family like cargo adaptor 2 | 22829 | NLGN4Y | neuroligin 4, Y-linked |
| 274 | BIN1 | bridging integrator 1 | 197358 | NLRC3 | NLR family CARD domain |
| | | | | | containing 3 |
| 51411 | BIN2 | bridging integrator 2 | 84166 | NLRC5 | NLR family CARD domain |
| | | | | | containing 5 |
| 330 | BIRC3 | baculoviral IAP repeat containing | 55655 | NLRP2 | NLR family pyrin domain |
| | | 3 | | | containing 2 |
| 332 | BIRC5 | baculoviral IAP repeat containing | 114548 | NLRP3 | NLR family pyrin domain |
| | | 5 | | | containing 3 |
| 640 | BLK | BLK proto-oncogene, Src family | 199713 | NLRP7 | NLR family pyrin domain |
| | | tyrosine kinase | | | containing 7 |
| 29760 | BLNK | B-cell linker | 8382 | NME5 | NME/NM23 family member 5 |
| 650 | BMP2 | bone morphogenetic protein 2 | 10874 | NMU | neuromedin U |
| 651 | BMP3 | bone morphogenetic protein 3 | 4837 | NNMT | nicotinamide N- |
| | | | | | methyltransferase |
| 652 | BMP4 | bone morphogenetic protein 4 | 64127 | NOD2 | nucleotide binding |
| | | | | | oligomerization domain |
| | | | | | containing 2 |
| 653 | BMP5 | bone morphogenetic protein 5 | 115677 | NOSTRIN | nitric oxide synthase trafficking |
| 655 | BMP7 | bone morphogenetic protein 7 | 4856 | NOV | nephroblastoma overexpressed |
| 168667 | BMPER | BMP binding endothelial | 4857 | NOVA1 | NOVA alternative splicing |
| | | regulator | | | regulator 1 |
| 658 | BMPR1B | bone morphogenetic protein | 50507 | NOX4 | NADPH oxidase 4 |
| | | receptor type 1B | | | |
| 660 | BMX | BMX non-receptor tyrosine | 4862 | NPAS2 | neuronal PAS domain protein 2 |
| | | kinase | | | |
| 54796 | BNC2 | basonuclin 2 | 100288332 | NPIPA5 | nuclear pore complex interacting |
| | | | | | protein family member A5 |
| 664 | BNIP3 | BCL2 interacting protein 3 | 440348 | NPIPB15 | nuclear pore complex interacting |
| | | | | | protein family member B15 |
| 149428 | BNIPL | BCL2 interacting protein like | 255743 | NPNT | nephronectin |
| 91653 | BOC | BOC cell adhesion associated, | 4881 | NPR1 | natriuretic peptide receptor 1 |
| | | oncogene regulated | | | |
| 669 | BPGM | bisphosphoglycerate mutase | 4886 | NPY1R | neuropeptide Y receptor Y1 |
| 92747 | BPIFB1 | BPI fold containing family B | 1728 | NQO1 | NAD(P)H quinone |
| | | member 1 | | | dehydrogenase 1 |
| 54836 | BSPRY | B-box and SPRY domain | 9971 | NR1H4 | nuclear receptor subfamily 1 |
| | | containing | | | group H member 4 |
| 684 | BST2 | bone marrow stromal cell antigen | 7025 | NR2F1 | nuclear receptor subfamily 2 |
| | | 2 | | | group F member 1 |
| 121551 | BTBD11 | BTB domain containing 11 | 4306 | NR3C2 | nuclear receptor subfamily 3 |
| | | | | | group C member 2 |
| 118663 | BTBD16 | BTB domain containing 16 | 3164 | NR4A1 | nuclear receptor subfamily 4 |
| | | | | | group A member 1 |
| 685 | BTC | betacellulin | 4929 | NR4A2 | nuclear receptor subfamily 4 |
| | | | | | group A member 2 |
| 7832 | BTG2 | BTG anti-proliferation factor 2 | 8013 | NR4A3 | nuclear receptor subfamily 4 |
| | | | | | group A member 3 |
| 695 | BTK | Bruton tyrosine kinase | 4897 | NRCAM | neuronal cell adhesion molecule |
| 699 | BUB1 | BUB1 mitotic checkpoint | 3084 | NRG1 | neuregulin 1 |
| | | serine/threonine kinase | | | |
| 701 | BUB1B | BUB1 mitotic checkpoint | 145957 | NRG4 | neuregulin 4 |
| | | serine/threonine kinase B | | | |
| 11149 | BVES | blood vessel epicardial | 203447 | NRK | Nik related kinase |
| | | substance | | | |
| 387638 | C10orf113 | chromosome 10 open reading | 8828 | NRP2 | neuropilin 2 |
| | | frame 113 | | | |
| 387695 | C10orf99 | chromosome 10 open reading | 9379 | NRXN2 | neurexin 2 |
| | | frame 99 | | | |
| 387763 | C11orf96 | chromosome 11 open reading | 27065 | NSG1 | neuronal vesicle trafficking |
| | | frame 96 | | | associated 1 |
| 115749 | C12orf56 | chromosome 12 open reading | 79730 | NSUN7 | NOP2/Sun RNA |
| | | frame 56 | | | methyltransferase family member |
| | | | | | 7 |
| 387882 | C12orf75 | chromosome 12 open reading | 4907 | NT5E | 5′-nucleotidase ecto |
| | | frame 75 | | | |
| 145407 | C14orf37 | chromosome 14 open reading | 4908 | NTF3 | neurotrophin 3 |
| | | frame 37 | | | |
| 84419 | C15orf48 | chromosome 15 open reading | 4909 | NTF4 | neurotrophin 4 |
| | | frame 48 | | | |
| 89927 | C16orf45 | chromosome 16 open reading | 59277 | NTN4 | netrin 4 |
| | | frame 45 | | | |
| 146556 | C16orf89 | chromosome 16 open reading | 22854 | NTNG1 | netrin G1 |
| | | frame 89 | | | |
| 400566 | C17orf97 | chromosome 17 open reading | 4915 | NTRK2 | neurotrophic receptor tyrosine |
| | | frame 97 | | | kinase 2 |
| 64073 | C19orf33 | chromosome 19 open reading | 4916 | NTRK3 | neurotrophic receptor tyrosine |
| | | frame 33 | | | kinase 3 |
| 55765 | C1orf106 | chromosome 1 open reading | 83540 | NUF2 | NUF2, NDC80 kinetochore |
| | | frame 106 | | | complex component |
| 79098 | C1orf116 | chromosome 1 open reading | 389643 | NUGGC | nuclear GTPase, germinal center |
| | | frame 116 | | | associated |
| 128346 | C1orf162 | chromosome 1 open reading | 23225 | NUP210 | nucleoporin 210 |
| | | frame 162 | | | |
| 148304 | C1orf74 | chromosome 1 open reading | 54830 | NUP62CL | nucleoporin 62 C-terminal like |
| | | frame 74 | | | |
| 712 | C1QA | complement C1q A chain | 26471 | NUPR1 | nuclear protein 1, transcriptional |
| | | | | | regulator |
| 713 | C1QB | complement C1q B chain | 51203 | NUSAP1 | nucleolar and spindle associated |
| | | | | | protein 1 |
| 714 | C1QC | complement C1q C chain | 57523 | NYNRIN | NYN domain and retroviral |
| | | | | | integrase containing |
| 10882 | C1QL1 | complement C1q like 1 | 4938 | OAS1 | 2′-5′-oligoadenylate synthetase 1 |
| 715 | C1R | complement C1r | 4939 | OAS2 | 2′-5′-oligoadenylate synthetase 2 |
| 716 | C1S | complement C1s | 4940 | OAS3 | 2′-5′-oligoadenylate synthetase 3 |
| 717 | C2 | complement C2 | 8638 | OASL | 2′-5′-oligoadenylate synthetase |
| | | | | | like |
| 84417 | C2orf40 | chromosome 2 open reading | 84033 | OBSCN | obscurin, cytoskeletal calmodulin |
| | | frame 40 | | | and titin-interacting RhoGEF |
| 348738 | C2orf48 | chromosome 2 open reading | 100506658 | OCLN | occludin |
| | | frame 48 | | | |
| 339804 | C2orf74 | chromosome 2 open reading | 266553 | OFCC1 | orofacial cleft 1 candidate 1 |
| | | frame 74 | | | |
| 718 | C3 | complement C3 | 4969 | OGN | osteoglycin |
| 719 | C3AR1 | complement C3a receptor 1 | 10439 | OLFM1 | olfactomedin 1 |
| 57415 | C3orf14 | chromosome 3 open reading | 93145 | OLFM2 | olfactomedin 2 |
| | | frame 14 | | | |
| 79669 | C3orf52 | chromosome 3 open reading | 10562 | OLFM4 | olfactomedin 4 |
| | | frame 52 | | | |
| 200844 | C3orf67 | chromosome 3 open reading | 283298 | OLFML1 | olfactomedin like 1 |
| | | frame 67 | | | |
| 720 | C4A | complement C4A (Rodgers blood | 25903 | OLFML2B | olfactomedin like 2B |
| | | group) | | | |
| 721 | C4B | complement C4B (Chido blood | 56944 | OLFML3 | olfactomedin like 3 |
| | | group) | | | |
| 55286 | C4orf19 | chromosome 4 open reading | 4973 | OLR1 | oxidized low density lipoprotein |
| | | frame 19 | | | receptor 1 |
| 389336 | C5orf46 | chromosome 5 open reading | 4958 | OMD | osteomodulin |
| | | frame 46 | | | |
| 729 | C6 | complement C6 | 4975 | OMP | olfactory marker protein |
| 647024 | C6orf132 | chromosome 6 open reading | 26219 | OR1J4 | olfactory receptor family 1 |
| | | frame 132 | | | subfamily J member 4 |
| 100996634 | C6orf183 | chromosome 6 open reading | 138882 | OR1N2 | olfactory receptor family 1 |
| | | frame 183 | | | subfamily N member 2 |
| 730 | C7 | complement C7 | 401427 | OR2A7 | olfactory receptor family 2 |
| | | | | | subfamily A member 7 |
| 100127983 | C8orf88 | chromosome 8 open reading | 390072 | OR52N4 | olfactory receptor family 52 |
| | | frame 88 | | | subfamily N member 4 |
| | | | | | (gene/pseudogene) |
| 771 | CA12 | carbonic anhydrase 12 | 120065 | OR5P2 | olfactory receptor family 5 |
| | | | | | subfamily P member 2 |
| 760 | CA2 | carbonic anhydrase 2 | 120066 | OR5P3 | olfactory receptor family 5 |
| | | | | | subfamily P member 3 |
| 762 | CA4 | carbonic anhydrase 4 | 10819 | OR7E14P | olfactory receptor family 7 |
| | | | | | subfamily E member 14 |
| | | | | | pseudogene |
| 767 | CA8 | carbonic anhydrase 8 | 26628 | OR7E47P | olfactory receptor family 7 |
| | | | | | subfamily E member 47 |
| | | | | | pseudogene |
| 768 | CA9 | carbonic anhydrase 9 | 79315 | OR7E91P | olfactory receptor family 7 |
| | | | | | subfamily E member 91 |
| | | | | | pseudogene |
| 81617 | CAB39L | calcium binding protein 39 like | 4998 | ORC1 | origin recognition complex |
| | | | | | subunit 1 |
| 26256 | CABYR | calcium binding tyrosine | 23594 | ORC6 | origin recognition complex |
| | | phosphorylation regulated | | | subunit 6 |
| 775 | CACNA1C | calcium voltage-gated channel | 5004 | ORM1 | orosomucoid 1 |
| | | subunit alpha1 C | | | |
| 776 | CACNA1D | calcium voltage-gated channel | 9180 | OSMR | oncostatin M receptor |
| | | subunit alpha1 D | | | |
| 8912 | CACNA1H | calcium voltage-gated channel | 130497 | OSR1 | odd-skipped related transciption |
| | | subunit alpha1 H | | | factor 1 |
| 781 | CACNA2D1 | calcium voltage-gated channel | 116039 | OSR2 | odd-skipped related transciption |
| | | auxiliary subunit alpha2delta 1 | | | factor 2 |
| 55799 | CACNA2D3 | calcium voltage-gated channel | 341277 | OVCH2 | ovochymase 2 |
| | | auxiliary subunit alpha2delta 3 | | | (gene/pseudogene) |
| 783 | CACNB2 | calcium voltage-gated channel | 5017 | OVOL1 | ovo like transcriptional repressor |
| | | auxiliary subunit beta 2 | | | 1 |
| 27092 | CACNG4 | calcium voltage-gated channel | 408186 | OVOS | ovostatin |
| | | auxiliary subunit gamma 4 | | | |
| 23705 | CADM1 | cell adhesion molecule 1 | 5019 | OXCT1 | 3-oxoacid CoA-transferase 1 |
| 57863 | CADM3 | cell adhesion molecule 3 | 165140 | OXER1 | oxoeicosanoid receptor 1 |
| 794 | CALB2 | calbindin 2 | 5023 | P2RX1 | purinergic receptor P2X 1 |
| 800 | CALD1 | caldesmon 1 | 27334 | P2RY10 | P2Y receptor family member 10 |
| 441168 | CALHM6 | calcium homeostasis modulator | 53829 | P2RY13 | purinergic receptor P2Y13 |
| | | family member 6 | | | |
| 55450 | CAMK2N1 | calcium/calmodulin dependent | 9934 | P2RY14 | purinergic receptor P2Y14 |
| | | protein kinase Il inhibitor 1 | | | |
| 814 | CAMK4 | calcium/calmodulin dependent | 55214 | P3H2 | prolyl 3-hydroxylase 2 |
| | | protein kinase IV | | | |
| 57662 | CAMSAP3 | calmodulin regulated spectrin | 10536 | P3H3 | prolyl 3-hydroxylase 3 |
| | | associated protein family | | | |
| | | member 3 | | | |
| 92291 | CAPN13 | calpain 13 | 283208 | P4HA3 | prolyl 4-hydroxylase subunit |
| | | | | | alpha 3 |
| 726 | CAPN5 | calpain 5 | 29943 | PADI1 | peptidyl arginine deiminase 1 |
| 827 | CAPN6 | calpain 6 | 51702 | PADI3 | peptidyl arginine deiminase 3 |
| 388743 | CAPN8 | calpain 8 | 389860 | PAGE2B | PAGE family member 2B |
| 10753 | CAPN9 | calpain 9 | 23022 | PALLD | palladin, cytoskeletal associated |
| | | | | | protein |
| 84290 | CAPNS2 | calpain small subunit 2 | 342979 | PALM3 | paralemmin 3 |
| 84433 | CARD11 | caspase recruitment domain | 54873 | PALMD | palmdelphin |
| | | family member 11 | | | |
| 440068 | CARD17 | caspase recruitment domain | 25891 | PAMR1 | peptidase domain containing |
| | | family member 17 | | | associated with muscle |
| | | | | | regeneration 1 |
| 146206 | CARMIL2 | capping protein regulator and | 5069 | PAPPA | pappalysin 1 |
| | | myosin 1 linker 2 | | | |
| 399726 | CASC10 | cancer susceptibility 10 | 164091 | PAQR7 | progestin and adipoQ receptor |
| | | | | | family member 7 |
| 23581 | CASP14 | caspase 14 | 84612 | PARD6B | par-6 family cell polarity regulator |
| | | | | | beta |
| 844 | CASQ1 | calsequestrin 1 | 25849 | PARM1 | prostate androgen-regulated |
| | | | | | mucin-like protein 1 |
| 845 | CASQ2 | calsequestrin 2 | 165631 | PARP15 | poly(ADP-ribose) polymerase |
| | | | | | family member 15 |
| 79820 | CATSPERB | cation channel sperm associated | 64098 | PARVG | parvin gamma |
| | | auxiliary subunit beta | | | |
| 857 | CAV1 | caveolin 1 | 197135 | PATL2 | PAT1 homolog 2 |
| 284119 | CAVIN1 | caveolae associated protein 1 | 5083 | PAX9 | paired box 9 |
| 8436 | CAVIN2 | caveolae associated protein 2 | 55872 | PBK | PDZ binding kinase |
| 23624 | CBLC | Cbl proto-oncogene C | 5097 | PCDH1 | protocadherin 1 |
| 84733 | CBX2 | chromobox 2 | 54510 | PCDH18 | protocadherin 18 |
| 285025 | CCDC141 | coiled-coil domain containing 141 | 57526 | PCDH19 | protocadherin 19 |
| 9720 | CCDC144A | coiled-coil domain containing | 5099 | PCDH7 | protocadherin 7 |
| | | 144A | | | |
| 284047 | CCDC144B | coiled-coil domain containing | 56126 | PCDHB10 | protocadherin beta 10 |
| | | 144B (pseudogene) | | | |
| 221262 | CCDC162P | coiled-coil domain containing | 56125 | PCDHB11 | protocadherin beta 11 |
| | | 162, pseudogene | | | |
| 728591 | CCDC169 | coiled-coil domain containing 169 | 57717 | PCDHB16 | protocadherin beta 16 |
| 84960 | CCDC183 | coiled-coil domain containing 183 | 54661 | PCDHB17P | protocadherin beta 17 |
| | | | | | pseudogene |
| 26112 | CCDC69 | coiled-coil domain containing 69 | 56133 | PCDHB2 | protocadherin beta 2 |
| 83987 | CCDC8 | coiled-coil domain containing 8 | 56132 | PCDHB3 | protocadherin beta 3 |
| 151887 | CCDC80 | coiled-coil domain containing 80 | 56131 | PCDHB4 | protocadherin beta 4 |
| 6356 | CCL11 | C-C motif chemokine ligand 11 | 26167 | PCDHB5 | protocadherin beta 5 |
| 6357 | CCL13 | C-C motif chemokine ligand 13 | 56130 | PCDHB6 | protocadherin beta 6 |
| 6358 | CCL14 | C-C motif chemokine ligand 14 | 56129 | PCDHB7 | protocadherin beta 7 |
| 6361 | CCL17 | C-C motif chemokine ligand 17 | 56128 | PCDHB8 | protocadherin beta 8 |
| 6362 | CCL18 | C-C motif chemokine ligand 18 | 56127 | PCDHB9 | protocadherin beta 9 |
| 6363 | CCL19 | C-C motif chemokine ligand 19 | 56104 | PCDHGB1 | protocadherin gamma subfamily |
| | | | | | B, 1 |
| 6347 | CCL2 | C-C motif chemokine ligand 2 | 5098 | PCDHGC3 | protocadherin gamma subfamily |
| | | | | | C, 3 |
| 6364 | CCL20 | C-C motif chemokine ligand 20 | 27445 | PCLO | piccolo presynaptic cytomatrix |
| | | | | | protein |
| 6366 | CCL21 | C-C motif chemokine ligand 21 | 5118 | PCOLCE | procollagen C-endopeptidase |
| | | | | | enhancer |
| 6368 | CCL23 | C-C motif chemokine ligand 23 | 26577 | PCOLCE2 | procollagen C-endopeptidase |
| | | | | | enhancer 2 |
| 6369 | CCL24 | C-C motif chemokine ligand 24 | 5121 | PCP4 | Purkinje cell protein 4 |
| 6348 | CCL3 | C-C motif chemokine ligand 3 | 654790 | PCP4L1 | Purkinje cell protein 4 like 1 |
| 6349 | CCL3L1 | C-C motif chemokine ligand 3 | 5122 | PCSK1 | proprotein convertase |
| | | like 1 | | | subtilisin/kexin type 1 |
| 6351 | CCL4 | C-C motif chemokine ligand 4 | 5046 | PCSK6 | proprotein convertase |
| | | | | | subtilisin/kexin type 6 |
| 388372 | CCL4L1 | C-C motif chemokine ligand 4 | 80380 | PDCD1LG2 | programmed cell death 1 ligand 2 |
| | | like 1 | | | |
| 9560 | CCL4L2 | C-C motif chemokine ligand 4 | 10846 | PDE10A | phosphodiesterase 10A |
| | | like 2 | | | |
| 6352 | CCL5 | C-C motif chemokine ligand 5 | 5136 | PDE1A | phosphodiesterase 1A |
| 6355 | CCL8 | C-C motif chemokine ligand 8 | 5137 | PDE1C | phosphodiesterase 1C |
| 890 | CCNA2 | cyclin A2 | 5138 | PDE2A | phosphodiesterase 2A |
| 891 | CCNB1 | cyclin B1 | 5139 | PDE3A | phosphodiesterase 3A |
| 9133 | CCNB2 | cyclin B2 | 5140 | PDE3B | phosphodiesterase 3B |
| 595 | CCND1 | cyclin D1 | 5142 | PDE4B | phosphodiesterase 4B |
| 898 | CCNE1 | cyclin E1 | 8654 | PDE5A | phosphodiesterase 5A |
| 9134 | CCNE2 | cyclin E2 | 27115 | PDE7B | phosphodiesterase 7B |
| 729230 | CCR2 | C-C motif chemokine receptor 2 | 8622 | PDE8B | phosphodiesterase 8B |
| 1233 | CCR4 | C-C motif chemokine receptor 4 | 5152 | PDE9A | phosphodiesterase 9A |
| 1236 | CCR7 | C-C motif chemokine receptor 7 | 56034 | PDGFC | platelet derived growth factor C |
| 1237 | CCR8 | C-C motif chemokine receptor 8 | 80310 | PDGFD | platelet derived growth factor D |
| 401145 | CCSER1 | coiled-coil serine rich protein 1 | 5156 | PDGFRA | platelet derived growth factor |
| | | | | | receptor alpha |
| 135228 | CD109 | CD109 molecule | 5159 | PDGFRB | platelet derived growth factor |
| | | | | | receptor beta |
| 929 | CD14 | CD14 molecule | 5157 | PDGFRL | platelet derived growth factor |
| | | | | | receptor like |
| 9332 | CD163 | CD163 molecule | 5166 | PDK4 | pyruvate dehydrogenase kinase |
| | | | | | 4 |
| 4064 | CD180 | CD180 molecule | 27295 | PDLIM3 | PDZ and LIM domain 3 |
| 930 | CD19 | CD19 molecule | 8572 | PDLIM4 | PDZ and LIM domain 4 |
| 911 | CD1C | CD1c molecule | 9260 | PDLIM7 | PDZ and LIM domain 7 |
| 913 | CD1E | CD1e molecule | 10630 | PDPN | podoplanin |
| 914 | CD2 | CD2 molecule | 10158 | PDZK1IP1 | PDZK1 interacting protein 1 |
| 4345 | CD200 | CD200 molecule | 23024 | PDZRN3 | PDZ domain containing ring |
| | | | | | finger 3 |
| 30835 | CD209 | CD209 molecule | 29951 | PDZRN4 | PDZ domain containing ring |
| | | | | | finger 4 |
| 933 | CD22 | CD22 molecule | 23089 | PEG10 | paternally expressed 10 |
| 100133941 | CD24 | CD24 molecule | 5187 | PER1 | period circadian regulator 1 |
| 919 | CD247 | CD247 molecule | 8863 | PER3 | period circadian regulator 3 |
| 57124 | CD248 | CD248 molecule | 64065 | PERP | PERP, TP53 apoptosis effector |
| 939 | CD27 | CD27 molecule | 5210 | PFKFB4 | 6-phosphofructo-2- |
| | | | | | kinase/fructose-2,6- |
| | | | | | biphosphatase 4 |
| 29126 | CD274 | CD274 molecule | 80162 | PGGHG | protein- |
| | | | | | glucosylgalactosylhydroxylysine |
| | | | | | glucosidase |
| 948 | CD36 | CD36 molecule | 5239 | PGM5 | phosphoglucomutase 5 |
| 951 | CD37 | CD37 molecule | 26227 | PHGDH | phosphoglycerate |
| | | | | | dehydrogenase |
| 952 | CD38 | CD38 molecule | 22822 | PHLDA1 | pleckstrin homology like domain |
| | | | | | family A member 1 |
| 915 | CD3D | CD3d molecule | 7262 | PHLDA2 | pleckstrin homology like domain |
| | | | | | family A member 2 |
| 916 | CD3E | CD3e molecule | 254295 | PHYHD1 | phytanoyl-CoA dioxygenase |
| | | | | | domain containing 1 |
| 917 | CD3G | CD3g molecule | 51050 | PI15 | peptidase inhibitor 15 |
| 959 | CD40LG | CD40 ligand | 221476 | PI16 | peptidase inhibitor 16 |
| 960 | CD44 | CD44 molecule (Indian blood | 5266 | PI3 | peptidase inhibitor 3 |
| | | group) | | | |
| 962 | CD48 | CD48 molecule | 728233 | PI4KAP1 | phosphatidylinositol 4-kinase |
| | | | | | alpha pseudogene 1 |
| 921 | CD5 | CD5 molecule | 375133 | PI4KAP2 | phosphatidylinositol 4-kinase |
| | | | | | alpha pseudogene 2 |
| 1043 | CD52 | CD52 molecule | 63895 | PIEZO2 | piezo type mechanosensitive ion |
| | | | | | channel component 2 |
| 963 | CD53 | CD53 molecule | 5284 | PIGR | polymeric immunoglobulin |
| | | | | | receptor |
| 923 | CD6 | CD6 molecule | 118788 | PIK3AP1 | phosphoinositide-3-kinase |
| | | | | | adaptor protein 1 |
| 969 | CD69 | CD69 molecule | 11040 | PIM2 | Pim-2 proto-oncogene, |
| | | | | | serine/threonine kinase |
| 924 | CD7 | CD7 molecule | 8395 | PIP5K1B | phosphatidylinositol-4-phosphate |
| | | | | | 5-kinase type 1 beta |
| 971 | CD72 | CD72 molecule | 8544 | PIR | pirin |
| 972 | CD74 | CD74 molecule | 5307 | PITX1 | paired like homeodomain 1 |
| 973 | CD79A | CD79a molecule | 5308 | PITX2 | paired like homeodomain 2 |
| 974 | CD79B | CD79b molecule | 168507 | PKD1L1 | polycystin 1 like 1, transient |
| | | | | | receptor potential channel |
| | | | | | interacting |
| 941 | CD80 | CD80 molecule | 91461 | PKDCC | protein kinase domain |
| | | | | | containing, cytoplasmic |
| 942 | CD86 | CD86 molecule | 5314 | PKHD1 | PKHD1, fibrocystin/polyductin |
| 925 | CD8A | CD8a molecule | 5569 | PKIA | cAMP-dependent protein kinase |
| | | | | | inhibitor alpha |
| 22918 | CD93 | CD93 molecule | 11142 | PKIG | cAMP-dependent protein kinase |
| | | | | | inhibitor gamma |
| 10225 | CD96 | CD96 molecule | 5317 | PKP1 | plakophilin 1 |
| 978 | CDA | cytidine deaminase | 5318 | PKP2 | plakophilin 2 |
| 991 | CDC20 | cell division cycle 20 | 11187 | PKP3 | plakophilin 3 |
| 8318 | CDC45 | cell division cycle 45 | 51365 | PLA1A | phospholipase A1 member A |
| 990 | CDC6 | cell division cycle 6 | 11145 | PLA2G16 | phospholipase A2 group XVI |
| 157313 | CDCA2 | cell division cycle associated 2 | 5320 | PLA2G2A | phospholipase A2 group IIA |
| 83879 | CDCA7 | cell division cycle associated 7 | 26279 | PLA2G2D | phospholipase A2 group IID |
| 55536 | CDCA7L | cell division cycle associated 7 | 64600 | PLA2G2F | phospholipase A2 group IIF |
| | | like | | | |
| 55143 | CDCA8 | cell division cycle associated 8 | 255189 | PLA2G4F | phospholipase A2 group IVF |
| 64866 | CDCP1 | CUB domain containing protein 1 | 7941 | PLA2G7 | phospholipase A2 group VII |
| 999 | CDH1 | cadherin 1 | 51316 | PLAC8 | placenta specific 8 |
| 1009 | CDH11 | cadherin 11 | 219348 | PLAC9 | placenta specific 9 |
| 28513 | CDH19 | cadherin 19 | 5325 | PLAGL1 | PLAG1 like zinc finger 1 |
| 1000 | CDH2 | cadherin 2 | 5327 | PLAT | plasminogen activator, tissue |
| | | | | | type |
| 60437 | CDH26 | cadherin 26 | 5328 | PLAU | plasminogen activator, urokinase |
| 1001 | CDH3 | cadherin 3 | 5329 | PLAUR | plasminogen activator, urokinase |
| | | | | | receptor |
| 1006 | CDH8 | cadherin 8 | 79887 | PLBD1 | phospholipase B domain |
| | | | | | containing 1 |
| 983 | CDK1 | cyclin dependent kinase 1 | 5330 | PLCB2 | phospholipase C beta 2 |
| 1029 | CDKN2A | cyclin dependent kinase inhibitor | 5332 | PLCB4 | phospholipase C beta 4 |
| | | 2A | | | |
| 1030 | CDKN2B | cyclin dependent kinase inhibitor | 5333 | PLCD1 | phospholipase C delta 1 |
| | | 2B | | | |
| 1033 | CDKN3 | cyclin dependent kinase inhibitor | 113026 | PLCD3 | phospholipase C delta 3 |
| | | 3 | | | |
| 1036 | CDO1 | cysteine dioxygenase type 1 | 84812 | PLCD4 | phospholipase C delta 4 |
| 50937 | CDON | cell adhesion associated, | 51196 | PLCE1 | phospholipase C epsilon 1 |
| | | oncogene regulated | | | |
| 634 | CEACAM1 | carcinoembryonic antigen related | 23007 | PLCH1 | phospholipase C eta 1 |
| | | cell adhesion molecule 1 | | | |
| 1048 | CEACAM5 | carcinoembryonic antigen related | 9651 | PLCH2 | phospholipase C eta 2 |
| | | cell adhesion molecule 5 | | | |
| 4680 | CEACAM6 | carcinoembryonic antigen related | 122618 | PLD4 | phospholipase D family member |
| | | cell adhesion molecule 6 | | | 4 |
| 1050 | CEBPA | CCAAT/enhancer binding protein | 5341 | PLEK | pleckstrin |
| | | alpha | | | |
| 1052 | CEBPD | CCAAT/enhancer binding protein | 26499 | PLEK2 | pleckstrin 2 |
| | | delta | | | |
| 27443 | CECR2 | CECR2, histone acetyl-lysine | 144100 | PLEKHA7 | pleckstrin homology domain |
| | | reader | | | containing A7 |
| 1056 | CEL | carboxyl ester lipase | 25894 | PLEKHG4 | pleckstrin homology and |
| | | | | | RhoGEF domain containing G4 |
| 10659 | CELF2 | CUGBP Elav-like family member | 55200 | PLEKHG6 | pleckstrin homology and |
| | | 2 | | | RhoGEF domain containing G6 |
| 9620 | CELSR1 | cadherin EGF LAG seven-pass | 57475 | PLEKHH1 | pleckstrin homology, MyTH4 and |
| | | G-type receptor 1 | | | FERM domain containing H1 |
| 1952 | CELSR2 | cadherin EGF LAG seven-pass | 130271 | PLEKHH2 | pleckstrin homology, MyTH4 and |
| | | G-type receptor 2 | | | FERM domain containing H2 |
| 1951 | CELSR3 | cadherin EGF LAG seven-pass | 84069 | PLEKHN1 | pleckstrin homology domain |
| | | G-type receptor 3 | | | containing N1 |
| 57214 | CEMIP | cell migration inducing | 79949 | PLEKHS1 | pleckstrin homology domain |
| | | hyaluronan binding protein | | | containing S1 |
| 1058 | CENPA | centromere protein A | 5346 | PLIN1 | perilipin 1 |
| 1063 | CENPF | centromere protein F | 729359 | PLIN4 | perilipin 4 |
| 79682 | CENPU | centromere protein U | 5347 | PLK1 | polo like kinase 1 |
| 201161 | CENPV | centromere protein V | 51090 | PLLP | plasmolipin |
| 55165 | CEP55 | centrosomal protein 55 | 5350 | PLN | phospholamban |
| 204219 | CERS3 | ceramide synthase 3 | 5352 | PLOD2 | procollagen-lysine,2-oxoglutarate |
| | | | | | 5-dioxygenase 2 |
| 79603 | CERS4 | ceramide synthase 4 | 5354 | PLP1 | proteolipid protein 1 |
| 1066 | CES1 | carboxylesterase 1 | 8611 | PLPP1 | phospholipid phosphatase 1 |
| 286464 | CFAP47 | cilia and flagella associated | 8612 | PLPP2 | phospholipid phosphatase 2 |
| | | protein 47 | | | |
| 629 | CFB | complement factor B | 196051 | PLPP4 | phospholipid phosphatase 4 |
| 1675 | CFD | complement factor D | 5357 | PLS1 | plastin 1 |
| 3075 | CFH | complement factor H | 57088 | PLSCR4 | phospholipid scramblase 4 |
| 3078 | CFHR1 | complement factor H related 1 | 84898 | PLXDC2 | plexin domain containing 2 |
| 3426 | CFI | complement factor I | 5365 | PLXNB3 | plexin B3 |
| 1073 | CFL2 | cofilin 2 | 10154 | PLXNC1 | plexin C1 |
| 5199 | CFP | complement factor properdin | 148811 | PM20D1 | peptidase M20 domain |
| | | | | | containing 1 |
| 57530 | CGN | cingulin | 56937 | PMEPA1 | prostate transmembrane protein, |
| | | | | | androgen induced 1 |
| 84952 | CGNL1 | cingulin like 1 | 83449 | PMFBP1 | polyamine modulated factor 1 |
| | | | | | binding protein 1 |
| 9023 | CH25H | cholesterol 25-hydroxylase | 5376 | PMP22 | peripheral myelin protein 22 |
| 1116 | CHI3L1 | chitinase 3 like 1 | 5380 | PMS2P2 | PMS1 homolog 2, mismatch |
| | | | | | repair system component |
| | | | | | pseudogene 2 |
| 1117 | CHI3L2 | chitinase 3 like 2 | 139728 | PNCK | pregnancy up-regulated |
| | | | | | nonubiquitous CaM kinase |
| 1118 | CHIT1 | chitinase 1 | 80339 | PNPLA3 | patatin like phospholipase |
| | | | | | domain containing 3 |
| 10752 | CHL1 | cell adhesion molecule L1 like | 127435 | PODN | podocan |
| 92421 | CHMP4C | charged multivesicular body | 50512 | PODXL2 | podocalyxin like 2 |
| | | protein 4C | | | |
| 63928 | CHP2 | calcineurin like EF-hand protein 2 | 79983 | POF1B | POF1B, actin binding protein |
| 91851 | CHRDL1 | chordin like 1 | 246721 | POLR2J2 | RNA polymerase Il subunit J2 |
| 25884 | CHRDL2 | chordin like 2 | 5446 | PON3 | paraoxonase 3 |
| 1129 | CHRM2 | cholinergic receptor muscarinic 2 | 64091 | POPDC2 | popeye domain containing 2 |
| 1140 | CHRNB1 | cholinergic receptor nicotinic beta | 10631 | POSTN | periostin |
| | | 1 subunit | | | |
| 148523 | CIART | circadian associated repressor of | 445582 | POTEE | POTE ankyrin domain family |
| | | transcription | | | member E |
| 4261 | CIITA | class II major histocompatibility | 728378 | POTEF | POTE ankyrin domain family |
| | | complex transactivator | | | member F |
| 8483 | CILP | cartilage intermediate layer | 404785 | POTEG | POTE ankyrin domain family |
| | | protein | | | member G |
| 150468 | CKAP2L | cytoskeleton associated protein 2 | 440915 | POTEKP | POTE ankyrin domain family |
| | | like | | | member K, pseudogene |
| 1152 | CKB | creatine kinase B | 5450 | POU2AF1 | POU class 2 associating factor 1 |
| 548596 | CKMT1A | creatine kinase, mitochondrial 1A | 25833 | POU2F3 | POU class 2 homeobox 3 |
| 1159 | CKMT1B | creatine kinase, mitochondrial 1B | 5460 | POU5F1 | POU class 5 homeobox 1 |
| 1163 | CKS1B | CDC28 protein kinase regulatory | 11281 | POU6F2 | POU class 6 homeobox 2 |
| | | subunit 1B | | | |
| 1164 | CKS2 | CDC28 protein kinase regulatory | 5468 | PPARG | peroxisome proliferator activated |
| | | subunit 2 | | | receptor gamma |
| 1178 | CLC | Charcot-Leyden crystal galectin | 10891 | PPARGC1A | PPARG coactivator 1 alpha |
| 9635 | CLCA2 | chloride channel accessory 2 | 5473 | PPBP | pro-platelet basic protein |
| 22802 | CLCA4 | chloride channel accessory 4 | 8499 | PPFIA2 | PTPRF interacting protein alpha |
| | | | | | 2 |
| 9076 | CLDN1 | claudin 1 | 8541 | PPFIA3 | PTPRF interacting protein alpha |
| | | | | | 3 |
| 5010 | CLDN11 | claudin 11 | 8495 | PPFIBP2 | PPFIA binding protein 2 |
| 100288814 | CLDN34 | claudin 34 | 5493 | PPL | periplakin |
| 1364 | CLDN4 | claudin 4 | 57460 | PPM1H | protein phosphatase, |
| | | | | | Mg2+/Mn2+ dependent 1H |
| 10462 | CLEC10A | C-type lectin domain containing | 10848 | PPP1R13L | protein phosphatase 1 regulatory |
| | | 10A | | | subunit 13 like |
| 6320 | CLEC11A | C-type lectin domain containing | 94274 | PPP1R14A | protein phosphatase 1 regulatory |
| | | 11A | | | inhibitor subunit 14A |
| 388512 | CLEC17A | C-type lectin domain containing | 81706 | PPP1R14C | protein phosphatase 1 regulatory |
| | | 17A | | | inhibitor subunit 14C |
| 9976 | CLEC2B | C-type lectin domain family 2 | 84152 | PPP1R1B | protein phosphatase 1 regulatory |
| | | member B | | | inhibitor subunit 1B |
| 7123 | CLEC3B | C-type lectin domain family 3 | 5507 | PPP1R3C | protein phosphatase 1 regulatory |
| | | member B | | | subunit 3C |
| 26253 | CLEC4E | C-type lectin domain family 4 | 55607 | PPP1R9A | protein phosphatase 1 regulatory |
| | | member E | | | subunit 9A |
| 64581 | CLEC7A | C-type lectin domain containing | 5521 | PPP2R2B | protein phosphatase 2 regulatory |
| | | 7A | | | subunit Bbeta |
| 1047 | CLGN | calmegin | 768206 | PRCD | photoreceptor disc component |
| 9022 | CLIC3 | chloride intracellular channel 3 | 5549 | PRELP | proline and arginine rich end |
| | | | | | leucine rich repeat protein |
| 54102 | CLIC6 | chloride intracellular channel 6 | 80243 | PREX2 | phosphatidylinositol-3,4,5- |
| | | | | | trisphosphate dependent Rac |
| | | | | | exchange factor 2 |
| 25999 | CLIP3 | CAP-Gly domain containing | 5551 | PRF1 | perforin 1 |
| | | linker protein 3 | | | |
| 79827 | CLMP | CXADR like membrane protein | 10216 | PRG4 | proteoglycan 4 |
| 116449 | CLNK | cytokine dependent | 144165 | PRICKLE1 | prickle planar cell polarity protein |
| | | hematopoietic cell linker | | | 1 |
| 63967 | CLSPN | claspin | 166336 | PRICKLE2 | prickle planar cell polarity protein |
| | | | | | 2 |
| 64084 | CLSTN2 | calsyntenin 2 | 5563 | PRKAA2 | protein kinase AMP-activated |
| | | | | | catalytic subunit alpha 2 |
| 1191 | CLU | clusterin | 5577 | PRKAR2B | protein kinase cAMP-dependent |
| | | | | | type II regulatory subunit beta |
| 1215 | CMA1 | chymase 1 | 5579 | PRKCB | protein kinase C beta |
| 8418 | CMAHP | cytidine monophospho-N- | 5588 | PRKCQ | protein kinase C theta |
| | | acetylneuraminic acid | | | |
| | | hydroxylase, pseudogene | | | |
| 129607 | CMPK2 | cytidine/uridine monophosphate | 5587 | PRKD1 | protein kinase D1 |
| | | kinase 2 | | | |
| 202333 | CMYA5 | cardiomyopathy associated 5 | 5592 | PRKG1 | protein kinase, cGMP- |
| | | | | | dependent, type I |
| 84518 | CNFN | cornifelin | 5616 | PRKY | protein kinase, Y-linked, |
| | | | | | pseudogene |
| 1259 | CNGA1 | cyclic nucleotide gated channel | 8842 | PROM1 | prominin 1 |
| | | alpha 1 | | | |
| 1264 | CNN1 | calponin 1 | 150696 | PROM2 | prominin 2 |
| 1272 | CNTN1 | contactin 1 | 5627 | PROS1 | protein S |
| 5067 | CNTN3 | contactin 3 | 55771 | PRR11 | proline rich 11 |
| 152330 | CNTN4 | contactin 4 | 79170 | PRR15L | proline rich 15 like |
| 23242 | COBL | cordon-bleu WH2 repeat protein | 80164 | PRR36 | proline rich 36 |
| 1690 | COCH | cochlin | 5396 | PRRX1 | paired related homeobox 1 |
| 1300 | COL10A1 | collagen type X alpha 1 chain | 5644 | PRSS1 | protease, serine 1 |
| 1301 | COL11A1 | collagen type XI alpha 1 chain | 8492 | PRSS12 | protease, serine 12 |
| 1303 | COL12A1 | collagen type XII alpha 1 chain | 10942 | PRSS21 | protease, serine 21 |
| 7373 | COL14A1 | collagen type XIV alpha 1 chain | 64063 | PRSS22 | protease, serine 22 |
| 1306 | COL15A1 | collagen type XV alpha 1 chain | 83886 | PRSS27 | protease, serine 27 |
| 1307 | COL16A1 | collagen type XVI alpha 1 chain | 5646 | PRSS3 | protease, serine 3 |
| 1308 | COL17A1 | collagen type XVII alpha 1 chain | 5652 | PRSS8 | protease, serine 8 |
| 80781 | COL18A1 | collagen type XVIII alpha 1 chain | 158471 | PRUNE2 | prune homolog 2 |
| 1310 | COL19A1 | collagen type XIX alpha 1 chain | 29968 | PSAT1 | phosphoserine aminotransferase |
| | | | | | 1 |
| 1277 | COL1A1 | collagen type I alpha 1 chain | 8000 | PSCA | prostate stem cell antigen |
| 1278 | COL1A2 | collagen type I alpha 2 chain | 100507463 | PSMB8-AS1 | PSMB8 antisense RNA 1 (head |
| | | | | | to head) |
| 81578 | COL21A1 | collagen type XXI alpha 1 chain | 5698 | PSMB9 | proteasome subunit beta 9 |
| 255631 | COL24A1 | collagen type XXIV alpha 1 chain | 9051 | PSTPIP1 | proline-serine-threonine |
| | | | | | phosphatase interacting protein 1 |
| 340267 | COL28A1 | collagen type XXVIII alpha 1 | 139411 | PTCHD1 | patched domain containing 1 |
| | | chain | | | |
| 1281 | COL3A1 | collagen type III alpha 1 chain | 5730 | PTGDS | prostaglandin D2 synthase |
| 1285 | COL4A3 | collagen type IV alpha 3 chain | 9536 | PTGES | prostaglandin E synthase |
| 1286 | COL4A4 | collagen type IV alpha 4 chain | 5740 | PTGIS | prostaglandin 12 synthase |
| 1287 | COL4A5 | collagen type IV alpha 5 chain | 22949 | PTGR1 | prostaglandin reductase 1 |
| 1288 | COL4A6 | collagen type IV alpha 6 chain | 5742 | PTGS1 | prostaglandin-endoperoxide |
| | | | | | synthase 1 |
| 1289 | COL5A1 | collagen type V alpha 1 chain | 5743 | PTGS2 | prostaglandin-endoperoxide |
| | | | | | synthase 2 |
| 1290 | COL5A2 | collagen type V alpha 2 chain | 5745 | PTH1R | parathyroid hormone 1 receptor |
| 50509 | COL5A3 | collagen type V alpha 3 chain | 5746 | PTH2R | parathyroid hormone 2 receptor |
| 1291 | COL6A1 | collagen type VI alpha 1 chain | 5744 | PTHLH | parathyroid hormone like |
| | | | | | hormone |
| 1292 | COL6A2 | collagen type VI alpha 2 chain | 5753 | PTK6 | protein tyrosine kinase 6 |
| 1293 | COL6A3 | collagen type VI alpha 3 chain | 5764 | PTN | pleiotrophin |
| 344875 | COL6A4P1 | collagen type VI alpha 4 | 5783 | PTPN13 | protein tyrosine phosphatase, |
| | | pseudogene 1 | | | non-receptor type 13 |
| 256076 | COL6A5 | collagen type VI alpha 5 chain | 26095 | PTPN20 | protein tyrosine phosphatase, |
| | | | | | non-receptor type 20 |
| 1294 | COL7A1 | collagen type VII alpha 1 chain | 26191 | PTPN22 | protein tyrosine phosphatase, |
| | | | | | non-receptor type 22 |
| 1295 | COL8A1 | collagen type VIII alpha 1 chain | 5788 | PTPRC | protein tyrosine phosphatase, |
| | | | | | receptor type C |
| 1296 | COL8A2 | collagen type VIII alpha 2 chain | 5790 | PTPRCAP | protein tyrosine phosphatase, |
| | | | | | receptor type C associated |
| | | | | | protein |
| 1297 | COL9A1 | collagen type IX alpha 1 chain | 5789 | PTPRD | protein tyrosine phosphatase, |
| | | | | | receptor type D |
| 1298 | COL9A2 | collagen type IX alpha 2 chain | 5794 | PTPRH | protein tyrosine phosphatase, |
| | | | | | receptor type H |
| 78989 | COLEC11 | collectin subfamily member 11 | 5797 | PTPRM | protein tyrosine phosphatase, |
| | | | | | receptor type M |
| 81035 | COLEC12 | collectin subfamily member 12 | 374462 | PTPRQ | protein tyrosine phosphatase, |
| | | | | | receptor type Q |
| 1311 | COMP | cartilage oligomeric matrix | 5801 | PTPRR | protein tyrosine phosphatase, |
| | | protein | | | receptor type R |
| 51226 | COPZ2 | coatomer protein complex | 10076 | PTPRU | protein tyrosine phosphatase, |
| | | subunit zeta 2 | | | receptor type U |
| 10699 | CORIN | corin, serine peptidase | 5803 | PTPRZ1 | protein tyrosine phosphatase, |
| | | | | | receptor type Z1 |
| 11151 | CORO1A | coronin 1A | 9232 | PTTG1 | pituitary tumor-transforming 1 |
| 4512 | COX1 | cytochrome c oxidase subunit I | 10744 | PTTG2 | pituitary tumor-transforming 2 |
| 4513 | COX2 | cytochrome c oxidase subunit II | 5806 | PTX3 | pentraxin 3 |
| 4514 | COX3 | cytochrome c oxidase III | 5816 | PVALB | parvalbumin |
| 1346 | COX7A1 | cytochrome c oxidase subunit | 7837 | PXDN | peroxidasin |
| | | 7A1 | | | |
| 1356 | CP | ceruloplasmin | 5831 | PYCR1 | pyrroline-5-carboxylate reductase |
| | | | | | 1 |
| 1359 | CPA3 | carboxypeptidase A3 | 5836 | PYGL | glycogen phosphorylase L |
| 51200 | CPA4 | carboxypeptidase A4 | 5837 | PYGM | glycogen phosphorylase, muscle |
| | | | | | associated |
| 27151 | CPAMD8 | C3 and PZP like, alpha-2- | 149628 | PYHIN1 | pyrin and HIN domain family |
| | | macroglobulin domain containing | | | member 1 |
| | | 8 | | | |
| 1363 | CPE | carboxypeptidase E | 25797 | QPCT | glutaminyl-peptide |
| | | | | | cyclotransferase |
| 79974 | CPED1 | cadherin like and PC-esterase | 84440 | RAB11FIP4 | RAB11 family interacting protein |
| | | domain containing 1 | | | 4 |
| 1368 | CPM | carboxypeptidase M | 376267 | RAB15 | RAB15, member RAS oncogene |
| | | | | | family |
| 27132 | CPNE7 | copine 7 | 401409 | RAB19 | RAB19, member RAS oncogene |
| | | | | | family |
| 1373 | CPS1 | carbamoyl-phosphate synthase 1 | 51715 | RAB23 | RAB23, member RAS oncogene |
| | | | | | family |
| 54504 | CPVL | carboxypeptidase, vitellogenic | 57111 | RAB25 | RAB25, member RAS oncogene |
| | | like | | | family |
| 56265 | CPXM1 | carboxypeptidase X, M14 family | 5874 | RAB27B | RAB27B, member RAS |
| | | member 1 | | | oncogene family |
| 119587 | CPXM2 | carboxypeptidase X, M14 family | 23682 | RAB38 | RAB38, member RAS oncogene |
| | | member 2 | | | family |
| 8532 | CPZ | carboxypeptidase Z | 5880 | RAC2 | Rac family small GTPase 2 |
| 1378 | CR1 | complement C3b/C4b receptor 1 | 5881 | RAC3 | Rac family small GTPase 3 |
| | | (Knops blood group) | | | |
| 1380 | CR2 | complement C3d receptor 2 | 8438 | RAD54L | RAD54 like |
| 1382 | CRABP2 | cellular retinoic acid binding | 135250 | RAET1E | retinoic acid early transcript 1E |
| | | protein 2 | | | |
| 283229 | CRACR2B | calcium release activated | 353091 | RAET1G | retinoic acid early transcript 1G |
| | | channel regulator 2B | | | |
| 90993 | CREB3L1 | cAMP responsive element | 154064 | RAET1L | retinoic acid early transcript 1L |
| | | binding protein 3 like 1 | | | |
| 1396 | CRIP1 | cysteine rich protein 1 | 5896 | RAG1 | recombination activating 1 |
| 83690 | CRISPLD1 | cysteine rich secretory protein | 10742 | RAI2 | retinoic acid induced 2 |
| | | LCCL domain containing 1 | | | |
| 83716 | CRISPLD2 | cysteine rich secretory protein | 57186 | RALGAPA2 | Ral GTPase activating protein |
| | | LCCL domain containing 2 | | | catalytic alpha subunit 2 |
| 9244 | CRLF1 | cytokine receptor like factor 1 | 10267 | RAMP1 | receptor activity modifying |
| | | | | | protein 1 |
| 54677 | CROT | carnitine O-octanoyltransferase | 202151 | RANBP3L | RAN binding protein 3 like |
| 55118 | CRTAC1 | cartilage acidic protein 1 | 5909 | RAP1GAP | RAP1 GTPase activating protein |
| 1410 | CRYAB | crystallin alpha B | 51195 | RAPGEFL1 | Rap guanine nucleotide |
| | | | | | exchange factor like 1 |
| 55057 | CRYBG2 | crystallin beta-gamma domain | 5915 | RARB | retinoic acid receptor beta |
| | | containing 2 | | | |
| 1428 | CRYM | crystallin mu | 5918 | RARRES1 | retinoic acid receptor responder |
| | | | | | 1 |
| 158511 | CSAG1 | chondrosarcoma associated | 5919 | RARRES2 | retinoic acid receptor responder |
| | | gene 1 | | | 2 |
| 1436 | CSF1R | colony stimulating factor 1 | 5920 | RARRES3 | retinoic acid receptor responder |
| | | receptor | | | 3 |
| 1438 | CSF2RA | colony stimulating factor 2 | 64926 | RASAL3 | RAS protein activator like 3 |
| | | receptor alpha subunit | | | |
| 1439 | CSF2RB | colony stimulating factor 2 | 51655 | RASD1 | ras related dexamethasone |
| | | receptor beta common subunit | | | induced 1 |
| 1441 | CSF3R | colony stimulating factor 3 | 158158 | RASEF | RAS and EF-hand domain |
| | | receptor | | | containing |
| 55790 | CSGALNACT1 | chondroitin sulfate N- | 10125 | RASGRP1 | RAS guanyl releasing protein 1 |
| | | acetylgalactosaminyltransferase | | | |
| | | 1 | | | |
| 114784 | CSMD2 | CUB and Sushi multiple domains | 10235 | RASGRP2 | RAS guanyl releasing protein 2 |
| | | 2 | | | |
| 1464 | CSPG4 | chondroitin sulfate proteoglycan | 387496 | RASL11A | RAS like family 11 member A |
| | | 4 | | | |
| 64651 | CSRNP1 | cysteine and serine rich nuclear | 65997 | RASL11B | RAS like family 11 member B |
| | | protein 1 | | | |
| 1465 | CSRP1 | cysteine and glycine rich protein | 166824 | RASSF6 | Ras association domain family |
| | | 1 | | | member 6 |
| 1469 | CST1 | cystatin SN | 9182 | RASSF9 | Ras association domain family |
| | | | | | member 9 |
| 1470 | CST2 | cystatin SA | 54033 | RBM11 | RNA binding motif protein 11 |
| 1474 | CST6 | cystatin E/M | 27303 | RBMS3 | RNA binding motif single |
| | | | | | stranded interacting protein 3 |
| 8530 | CST7 | cystatin F | 5947 | RBP | retinol binding protein 1 |
| 1475 | CSTA | cystatin A | 5950 | RBP4 | retinol binding protein 4 |
| 1476 | CSTB | cystatin B | 83758 | RBP5 | retinol binding protein 5 |
| 441294 | CTAGE15 | CTAGE family member 15 | 116362 | RBP7 | retinol binding protein 7 |
| 1490 | CTGF | connective tissue growth factor | 348093 | RBPMS2 | RNA binding protein with multiple |
| | | | | | splicing 2 |
| 115908 | CTHRC1 | collagen triple helix repeat | 1827 | RCAN1 | regulator of calcineurin 1 |
| | | containing 1 | | | |
| 1493 | CTLA4 | cytotoxic T-lymphocyte | 10231 | RCAN2 | regulator of calcineurin 2 |
| | | associated protein 4 | | | |
| 1510 | CTSE | cathepsin E | 57333 | RCN3 | reticulocalbin 3 |
| 1511 | CTSG | cathepsin G | 9401 | RECQL4 | RecQ like helicase 4 |
| 1512 | CTSH | cathepsin H | 65055 | REEP1 | receptor accessory protein 1 |
| 1513 | CTSK | cathepsin K | 92840 | REEP6 | receptor accessory protein 6 |
| 1520 | CTSS | cathepsin S | 5649 | RELN | reelin |
| 1515 | CTSV | cathepsin V | 5972 | REN | renin |
| 1521 | CTSW | cathepsin W | 646396 | REREP3 | arginine-glutamic acid dipeptide |
| | | | | | repeats pseudogene 3 |
| 83992 | CTTNBP2 | cortactin binding protein 2 | 85004 | RERG | RAS like estrogen regulated |
| | | | | | growth inhibitor |
| 8029 | CUBN | cubilin | 79785 | RERGL | RERG like |
| 80157 | CWH43 | cell wall biogenesis 43 C-terminal | 57139 | RGL3 | ral guanine nucleotide |
| | | homolog | | | dissociation stimulator like 3 |
| 6376 | CX3CL1 | C-X3-C motif chemokine ligand 1 | 9104 | RGN | regucalcin |
| 1525 | CXADR | CXADR, Ig-like cell adhesion | 5996 | RGS1 | regulator of G protein signaling 1 |
| | | molecule | | | |
| 2919 | CXCL1 | C-X-C motif chemokine ligand 1 | 8786 | RGS11 | regulator of G protein signaling |
| | | | | | 11 |
| 3627 | CXCL10 | C-X-C motif chemokine ligand 10 | 6003 | RGS13 | regulator of G protein signaling |
| | | | | | 13 |
| 6373 | CXCL11 | C-X-C motif chemokine ligand 11 | 6004 | RGS16 | regulator of G protein signaling |
| | | | | | 16 |
| 6387 | CXCL12 | C-X-C motif chemokine ligand 12 | 26575 | RGS17 | regulator of G protein signaling |
| | | | | | 17 |
| 10563 | CXCL13 | C-X-C motif chemokine ligand 13 | 5997 | RGS2 | regulator of G protein signaling 2 |
| 9547 | CXCL14 | C-X-C motif chemokine ligand 14 | 26166 | RGS22 | regulator of G protein signaling |
| | | | | | 22 |
| 284340 | CXCL17 | C-X-C motif chemokine ligand 17 | 5999 | RGS4 | regulator of G protein signaling 4 |
| 2920 | CXCL2 | C-X-C motif chemokine ligand 2 | 8490 | RGS5 | regulator of G protein signaling 5 |
| 6372 | CXCL6 | C-X-C motif chemokine ligand 6 | 9628 | RGS6 | regulator of G protein signaling 6 |
| 3576 | CXCL8 | C-X-C motif chemokine ligand 8 | 8787 | RGS9 | regulator of G protein signaling 9 |
| 4283 | CXCL9 | C-X-C motif chemokine ligand 9 | 54933 | RHBDL2 | rhomboid like 2 |
| 3577 | CXCR1 | C-X-C motif chemokine receptor | 57127 | RHBG | Rh family B glycoprotein |
| | | 1 | | | (gene/pseudogene) |
| 7852 | CXCR4 | C-X-C motif chemokine receptor | 51458 | RHCG | Rh family C glycoprotein |
| | | 4 | | | |
| 10663 | CXCR6 | C-X-C motif chemokine receptor | 440712 | RHEX | regulator of hemoglobinization |
| | | 6 | | | and erythroid cell expansion |
| 55086 | CXorf57 | chromosome X open reading | 388 | RHOB | ras homolog family member B |
| | | frame 57 | | | |
| 1528 | CYB5A | cytochrome b5 type A | 29984 | RHOD | ras homolog family member D |
| 1536 | CYBB | cytochrome b-245 beta chain | 54509 | RHOF | ras homolog family member F, |
| | | | | | filopodia associated |
| 79901 | CYBRD1 | cytochrome b reductase 1 | 58480 | RHOU | ras homolog family member U |
| 1545 | CYP1B1 | cytochrome P450 family 1 | 171177 | RHOV | ras homolog family member V |
| | | subfamily B member 1 | | | |
| 1590 | CYP21A1P | cytochrome P450 family 21 | 114822 | RHPN1 | rhophilin Rho GTPase binding |
| | | subfamily A member 1, | | | protein 1 |
| | | pseudogene | | | |
| 1589 | CYP21A2 | cytochrome P450 family 21 | 85415 | RHPN2 | rhophilin Rho GTPase binding |
| | | subfamily A member 2 | | | protein 2 |
| 1591 | CYP24A1 | cytochrome P450 family 24 | 26150 | RIBC2 | RIB43A domain with coiled-coils |
| | | subfamily A member 1 | | | 2 |
| 1593 | CYP27A1 | cytochrome P450 family 27 | 79608 | RIC3 | RIC3 acetylcholine receptor |
| | | subfamily A member 1 | | | chaperone |
| 1562 | CYP2C18 | cytochrome P450 family 2 | 9699 | RIMS2 | regulating synaptic membrane |
| | | subfamily C member 18 | | | exocytosis 2 |
| 1557 | CYP2C19 | cytochrome P450 family 2 | 54101 | RIPK4 | receptor interacting |
| | | subfamily C member 19 | | | serine/threonine kinase 4 |
| 1558 | CYP2C8 | cytochrome P450 family 2 | 9750 | RIPOR2 | RHO family interacting cell |
| | | subfamily C member 8 | | | polarization regulator 2 |
| 1559 | CYP2C9 | cytochrome P450 family 2 | 125050 | RN7SK | RNA, 7SK small nuclear |
| | | subfamily C member 9 | | | |
| 1573 | CYP2J2 | cytochrome P450 family 2 | 6029 | RN7SL1 | RNA, 7SL, cytoplasmic 1 |
| | | subfamily J member 2 | | | |
| 29785 | CYP2S1 | cytochrome P450 family 2 | 378706 | RN7SL2 | RNA, 7SL, cytoplasmic 2 |
| | | subfamily S member 1 | | | |
| 51302 | CYP39A1 | cytochrome P450 family 39 | 6035 | RNASE1 | ribonuclease A family member 1, |
| | | subfamily A member 1 | | | pancreatic |
| 1577 | CYP3A5 | cytochrome P450 family 3 | 6036 | RNASE2 | ribonuclease A family member 2 |
| | | subfamily A member 5 | | | |
| 1580 | CYP4B1 | cytochrome P450 family 4 | 6038 | RNASE4 | ribonuclease A family member 4 |
| | | subfamily B member 1 | | | |
| 57834 | CYP4F11 | cytochrome P450 family 4 | 6039 | RNASE6 | ribonuclease A family member k6 |
| | | subfamily F member 11 | | | |
| 66002 | CYP4F12 | cytochrome P450 family 4 | 84659 | RNASE7 | ribonuclease A family member 7 |
| | | subfamily F member 12 | | | |
| 126410 | CYP4F22 | cytochrome P450 family 4 | 79589 | RNF128 | ring finger protein 128, E3 |
| | | subfamily F member 22 | | | ubiquitin protein ligase |
| 4051 | CYP4F3 | cytochrome P450 family 4 | 57484 | RNF150 | ring finger protein 150 |
| | | subfamily F member 3 | | | |
| 11283 | CYP4F8 | cytochrome P450 family 4 | 285671 | RNF180 | ring finger protein 180 |
| | | subfamily F member 8 | | | |
| 260293 | CYP4X1 | cytochrome P450 family 4 | 80352 | RNF39 | ring finger protein 39 |
| | | subfamily X member 1 | | | |
| 199974 | CYP4Z1 | cytochrome P450 family 4 | 54894 | RNF43 | ring finger protein 43 |
| | | subfamily Z member 1 | | | |
| 3491 | CYR61 | cysteine rich angiogenic inducer | 84900 | RNFT2 | ring finger protein, |
| | | 61 | | | transmembrane 2 |
| 84418 | CYSTM1 | cysteine rich transmembrane | 26824 | RNU11 | RNA, U11 small nuclear |
| | | module containing 1 | | | |
| 4519 | CYTB | cytochrome b | 267010 | RNU12 | RNA, U12 small nuclear |
| 27128 | CYTH4 | cytohesin 4 | 26855 | RNU2-2P | RNA, U2 small nuclear 2, |
| | | | | | pseudogene |
| 9595 | CYTIP | cytohesin 1 interacting protein | 26835 | RNU4-1 | RNA, U4 small nuclear 1 |
| 54360 | CYTL1 | cytokine like 1 | 26834 | RNU4-2 | RNA, U4 small nuclear 2 |
| 23500 | DAAM2 | dishevelled associated activator | 100151683 | RNU4ATAC | RNA, U4atac small nuclear (U12- |
| | | of morphogenesis 2 | | | dependent splicing) |
| 51339 | DACT1 | dishevelled binding antagonist of | 26831 | RNU5A-1 | RNA, U5A small nuclear 1 |
| | | beta catenin 1 | | | |
| 147906 | DACT3 | dishevelled binding antagonist of | 26832 | RNU5B-1 | RNA, U5B small nuclear 1 |
| | | beta catenin 3 | | | |
| 1612 | DAPK1 | death associated protein kinase | 26829 | RNU5E-1 | RNA, U5E small nuclear 1 |
| | | 1 | | | |
| 92196 | DAPL1 | death associated protein like 1 | 6084 | RNY1 | RNA, Ro-associated Y1 |
| 341019 | DCDC1 | doublecortin domain containing 1 | 6092 | ROBO2 | roundabout guidance receptor 2 |
| 8642 | DCHS1 | dachsous cadherin-related 1 | 4919 | ROR1 | receptor tyrosine kinase like |
| | | | | | orphan receptor 1 |
| 9201 | DCLK1 | doublecortin like kinase 1 | 4920 | ROR2 | receptor tyrosine kinase like |
| | | | | | orphan receptor 2 |
| 166614 | DCLK2 | doublecortin like kinase 2 | 56969 | RPL23AP32 | ribosomal protein L23a |
| | | | | | pseudogene 32 |
| 1634 | DCN | decorin | 6170 | RPL39 | ribosomal protein L39 |
| 54541 | DDIT4 | DNA damage inducible transcript | 116832 | RPL39L | ribosomal protein L39 like |
| | | 4 | | | |
| 4921 | DDR2 | discoidin domain receptor | 6133 | RPL9 | ribosomal protein L9 |
| | | tyrosine kinase 2 | | | |
| 8653 | DDX3Y | DEAD-box helicase 3, Y-linked | 85495 | RPPH1 | ribonuclease P RNA component |
| | | | | | H1 |
| 55510 | DDX43 | DEAD-box helicase 43 | 6232 | RPS27 | ribosomal protein S27 |
| 55601 | DDX60 | DExD/H-box helicase 60 | 6192 | RPS4Y1 | ribosomal protein S4, Y-linked 1 |
| 1672 | DEFB1 | defensin beta 1 | 6236 | RRAD | RRAD, Ras related glycolysis |
| | | | | | inhibitor and calcium channel |
| | | | | | regulator |
| 123099 | DEGS2 | delta 4-desaturase, sphingolipid | 6241 | RRM2 | ribonucleotide reductase |
| | | 2 | | | regulatory subunit M2 |
| 27147 | DENND2A | DENN domain containing 2A | 653390 | RRN3P2 | RRN3 homolog, RNA |
| | | | | | polymerase I transcription factor |
| | | | | | pseudogene 2 |
| 55635 | DEPDC1 | DEP domain containing 1 | 91543 | RSAD2 | radical S-adenosyl methionine |
| | | | | | domain containing 2 |
| 11067 | DEPP1 | DEPP1, autophagy regulator | 89765 | RSPH1 | radial spoke head 1 homolog |
| 64798 | DEPTOR | DEP domain containing MTOR | 84870 | RSPO3 | R-spondin 3 |
| | | interacting protein | | | |
| 1674 | DES | desmin | 65078 | RTN4R | reticulon 4 receptor |
| 84649 | DGAT2 | diacylglycerol O-acyltransferase | 64108 | RTP4 | receptor transporter protein 4 |
| | | 2 | | | |
| 100874048 | DGUOK-AS1 | DGUOK antisense RNA 1 | 80183 | RUBCNL | RUN and cysteine rich domain |
| | | | | | containing beclin 1 interacting |
| | | | | | protein like |
| 1718 | DHCR24 | 24-dehydrocholesterol reductase | 80215 | RUNX1-IT1 | RUNX1 intronic transcript 1 |
| 1717 | DHCR7 | 7-dehydrocholesterol reductase | 862 | RUNX1T1 | RUNX1 translocation partner 1 |
| 147015 | DHRS13 | dehydrogenase/reductase 13 | 6262 | RYR2 | ryanodine receptor 2 |
| 10202 | DHRS2 | dehydrogenase/reductase 2 | 6263 | RYR3 | ryanodine receptor 3 |
| 9249 | DHRS3 | dehydrogenase/reductase 3 | 6281 | S100A10 | S100 calcium binding protein |
| | | | | | A10 |
| 81624 | DIAPH3 | diaphanous related formin 3 | 6283 | S100A12 | S100 calcium binding protein |
| | | | | | A12 |
| 1734 | DIO2 | iodothyronine deiodinase 2 | 57402 | S100A14 | S100 calcium binding protein |
| | | | | | A14 |
| 85458 | DIXDC1 | DIX domain containing 1 | 140576 | S100A16 | S100 calcium binding protein |
| | | | | | A16 |
| 22943 | DKK1 | dickkopf WNT signaling pathway | 6273 | S100A2 | S100 calcium binding protein A2 |
| | | inhibitor 1 | | | |
| 9787 | DLGAP5 | DLG associated protein 5 | 6274 | S100A3 | S100 calcium binding protein A3 |
| 28514 | DLL1 | delta like canonical Notch ligand | 6275 | S100A4 | S100 calcium binding protein A4 |
| | | 1 | | | |
| 1749 | DLX5 | distal-less homeobox 5 | 6277 | S100A6 | S100 calcium binding protein A6 |
| 1755 | DMBT1 | deleted in malignant brain tumors | 6278 | S100A7 | S100 calcium binding protein A7 |
| | | 1 | | | |
| 1756 | DMD | dystrophin | 6279 | S100A8 | S100 calcium binding protein A8 |
| 29958 | DMGDH | dimethylglycine dehydrogenase | 6280 | S100A9 | S100 calcium binding protein A9 |
| 93099 | DMKN | dermokine | 6285 | S100B | S100 calcium binding protein B |
| 63951 | DMRTA1 | DMRT like family A1 | 6286 | S100P | S100 calcium binding protein P |
| 1767 | DNAH5 | dynein axonemal heavy chain 5 | 1901 | S1PR1 | sphingosine-1-phosphate |
| | | | | | receptor 1 |
| 11080 | DNAJB4 | DnaJ heat shock protein family | 6288 | SAA1 | serum amyloid A1 |
| | | (Hsp40) member B4 | | | |
| 25822 | DNAJB5 | DnaJ heat shock protein family | 6299 | SALL1 | spalt like transcription factor 1 |
| | | (Hsp40) member B5 | | | |
| 7802 | DNALI1 | dynein axonemal light | 57167 | SALL4 | spalt like transcription factor 4 |
| | | intermediate chain 1 | | | |
| 1776 | DNASE1L3 | deoxyribonuclease 1 like 3 | 154075 | SAMD3 | sterile alpha motif domain |
| | | | | | containing 3 |
| 1759 | DNM1 | dynamin 1 | 54809 | SAMD9 | sterile alpha motif domain |
| | | | | | containing 9 |
| 1789 | DNMT3B | DNA methyltransferase 3 beta | 219285 | SAMD9L | sterile alpha motif domain |
| | | | | | containing 9 like |
| 8447 | DOC2B | double C2 domain beta | 64092 | SAMSN1 | SAM domain, SH3 domain and |
| | | | | | nuclear localization signals 1 |
| 55619 | DOCK10 | dedicator of cytokinesis 10 | 54440 | SASH3 | SAM and SH3 domain containing |
| | | | | | 3 |
| 1794 | DOCK2 | dedicator of cytokinesis 2 | 374897 | SBSN | suprabasin |
| 1795 | DOCK3 | dedicator of cytokinesis 3 | 157869 | SBSPON | somatomedin B and |
| | | | | | thrombospondin type 1 domain |
| | | | | | containing |
| 1803 | DPP4 | dipeptidyl peptidase 4 | 51435 | SCARA3 | scavenger receptor class A |
| | | | | | member 3 |
| 1805 | DPT | dermatopontin | 286133 | SCARA5 | scavenger receptor class A |
| | | | | | member 5 |
| 283417 | DPY19L2 | dpy-19 like 2 | 91179 | SCARF2 | scavenger receptor class F |
| | | | | | member 2 |
| 1809 | DPYSL3 | dihydropyrimidinase like 3 | 677780 | SCARNA11 | small Cajal body-specific RNA 11 |
| 165545 | DQX1 | DEAQ-box RNA dependent | 692149 | SCARNA14 | small Cajal body-specific RNA 14 |
| | | ATPase 1 | | | |
| 51233 | DRICH1 | aspartate rich 1 | 677778 | SCARNA15 | small Cajal body-specific RNA 15 |
| 1824 | DSC2 | desmocollin 2 | 677765 | SCARNA18 | small Cajal body-specific RNA 18 |
| 1825 | DSC3 | desmocollin 3 | 677681 | SCARNA20 | small Cajal body-specific RNA 20 |
| 101927718 | DSG1-AS1 | DSG1 antisense RNA 1 | 677763 | SCARNA21 | small Cajal body-specific RNA 21 |
| 1829 | DSG2 | desmoglein 2 | 677770 | SCARNA22 | small Cajal body-specific RNA 22 |
| 1830 | DSG3 | desmoglein 3 | 677773 | SCARNA23 | small Cajal body-specific RNA 23 |
| 1832 | DSP | desmoplakin | 677679 | SCARNA3 | small Cajal body-specific RNA 3 |
| 401124 | DTHD1 | death domain containing 1 | 677771 | SCARNA4 | small Cajal body-specific RNA 4 |
| 1837 | DTNA | dystrobrevin alpha | 677772 | SCARNA6 | small Cajal body-specific RNA 6 |
| 23220 | DTX4 | deltex E3 ubiquitin ligase 4 | 677776 | SCARNA8 | small Cajal body-specific RNA 8 |
| 53905 | DUOX1 | dual oxidase 1 | 51097 | SCCPDH | saccharopine dehydrogenase |
| | | | | | (putative) |
| 50506 | DUOX2 | dual oxidase 2 | 6319 | SCD | stearoyl-CoA desaturase |
| 90527 | DUOXA1 | dual oxidase maturation factor 1 | 8796 | SCEL | sciellin |
| 405753 | DUOXA2 | dual oxidase maturation factor 2 | 7857 | SCG2 | secretogranin II |
| 1843 | DUSP1 | dual specificity phosphatase 1 | 6447 | SCG5 | secretogranin V |
| 1844 | DUSP2 | dual specificity phosphatase 2 | 85477 | SCIN | scinderin |
| 1847 | DUSP5 | dual specificity phosphatase 5 | 256380 | SCML4 | Scm polycomb group protein like |
| | | | | | 4 |
| 1852 | DUSP9 | dual specificity phosphatase 9 | 6326 | SCN2A | sodium voltage-gated channel |
| | | | | | alpha subunit 2 |
| 1780 | DYNC111 | dynein cytoplasmic 1 | 6328 | SCN3A | sodium voltage-gated channel |
| | | intermediate chain 1 | | | alpha subunit 3 |
| 1869 | E2F1 | E2F transcription factor 1 | 6330 | SCN4B | sodium voltage-gated channel |
| | | | | | beta subunit 4 |
| 144455 | E2F7 | E2F transcription factor 7 | 6331 | SCN5A | sodium voltage-gated channel |
| | | | | | alpha subunit 5 |
| 79733 | E2F8 | E2F transcription factor 8 | 6332 | SCN7A | sodium voltage-gated channel |
| | | | | | alpha subunit 7 |
| 1879 | EBF1 | early B-cell factor 1 | 6334 | SCN8A | sodium voltage-gated channel |
| | | | | | alpha subunit 8 |
| 79746 | ECHDC3 | enoyl-CoA hydratase domain | 6335 | SCN9A | sodium voltage-gated channel |
| | | containing 3 | | | alpha subunit 9 |
| 1893 | ECM1 | extracellular matrix protein 1 | 6337 | SCNN1A | sodium channel epithelial 1 alpha |
| | | | | | subunit |
| 1842 | ECM2 | extracellular matrix protein 2 | 6338 | SCNN1B | sodium channel epithelial 1 beta |
| | | | | | subunit |
| 1894 | ECT2 | epithelial cell transforming 2 | 6340 | SCNN1G | sodium channel epithelial 1 |
| | | | | | gamma subunit |
| 128178 | EDARADD | EDAR associated death domain | 11341 | SCRG1 | stimulator of chondrogenesis 1 |
| 10085 | EDIL3 | EGF like repeats and discoidin | 57758 | SCUBE2 | signal peptide, CUB domain and |
| | | domains 3 | | | EGF like domain containing 2 |
| 1906 | EDN1 | endothelin 1 | 222663 | SCUBE3 | signal peptide, CUB domain and |
| | | | | | EGF like domain containing 3 |
| 1907 | EDN2 | endothelin 2 | 6382 | SDC1 | syndecan 1 |
| 1909 | EDNRA | endothelin receptor type A | 6383 | SDC2 | syndecan 2 |
| 1910 | EDNRB | endothelin receptor type B | 221935 | SDK1 | sidekick cell adhesion molecule 1 |
| 1917 | EEF1A2 | eukaryotic translation elongation | 93517 | SDR42E1 | short chain |
| | | factor 1 alpha 2 | | | dehydrogenase/reductase family |
| | | | | | 42E, member 1 |
| 2202 | EFEMP1 | EGF containing fibulin | 6398 | SECTM1 | secreted and transmembrane 1 |
| | | extracellular matrix protein 1 | | | |
| 1942 | EFNA1 | ephrin A1 | 6401 | SELE | selectin E |
| 1947 | EFNB1 | ephrin B1 | 8991 | SELENBP1 | selenium binding protein 1 |
| 25975 | EGFL6 | EGF like domain multiple 6 | 140606 | SELENOM | selenoprotein M |
| 112399 | EGLN3 | egl-9 family hypoxia inducible | 6414 | SELENOP | selenoprotein P |
| | | factor 3 | | | |
| 1958 | EGR1 | early growth response 1 | 6402 | SELL | selectin L |
| 1959 | EGR2 | early growth response 2 | 6403 | SELP | selectin P |
| 1960 | EGR3 | early growth response 3 | 6404 | SELPLG | selectin P ligand |
| 30846 | EHD2 | EH domain containing 2 | 10371 | SEMA3A | semaphorin 3A |
| 26298 | EHF | ETS homologous factor | 7869 | SEMA3B | semaphorin 3B |
| 493861 | EID3 | EP300 interacting inhibitor of | 223117 | SEMA3D | semaphorin 3D |
| | | differentiation 3 | | | |
| 9086 | EIF1AY | eukaryotic translation initiation | 9723 | SEMA3E | semaphorin 3E |
| | | factor 1A, Y-linked | | | |
| 8663 | EIF3C | eukaryotic translation initiation | 6405 | SEMA3F | semaphorin 3F |
| | | factor 3 subunit C | | | |
| 1999 | ELF3 | E74 like ETS transcription factor | 10509 | SEMA4B | semaphorin 4B |
| | | 3 | | | |
| 2001 | ELF5 | E74 like ETS transcription factor | 9037 | SEMA5A | semaphorin 5A |
| | | 5 | | | |
| 2003 | ELK2AP | ELK2A, member of ETS | 57556 | SEMA6A | semaphorin 6A |
| | | oncogene family, pseudogene | | | |
| 79767 | ELMO3 | engulfment and cell motility 3 | 8482 | SEMA7A | semaphorin 7A (John Milton |
| | | | | | Hagen blood group) |
| 2006 | ELN | elastin | 347735 | SERINC2 | serine incorporator 2 |
| 79071 | ELOVL6 | ELOVL fatty acid elongase 6 | 5265 | SERPINA1 | serpin family A member 1 |
| 51705 | EMCN | endomucin | 12 | SERPINA3 | serpin family A member 3 |
| 11117 | EMILIN1 | elastin microfibril interfacer 1 | 5275 | SERPINB13 | serpin family B member 13 |
| 2009 | EML1 | echinoderm microtubule | 5055 | SERPINB2 | serpin family B member 2 |
| | | associated protein like 1 | | | |
| 161436 | EML5 | echinoderm microtubule | 6317 | SERPINB3 | serpin family B member 3 |
| | | associated protein like 5 | | | |
| 2012 | EMP1 | epithelial membrane protein 1 | 6318 | SERPINB4 | serpin family B member 4 |
| 2018 | EMX2 | empty spiracles homeobox 2 | 5268 | SERPINB5 | serpin family B member 5 |
| 5167 | ENPP1 | ectonucleotide | 5054 | SERPINE1 | serpin family E member 1 |
| | | pyrophosphatase/phosphodiesterase | | | |
| | | 1 | | | |
| 59084 | ENPP5 | ectonucleotide | 5270 | SERPINE2 | serpin family E member 2 |
| | | pyrophosphatase/phosphodiesterase | | | |
| | | 5 (putative) | | | |
| 956 | ENTPD3 | ectonucleoside triphosphate | 5176 | SERPINF1 | serpin family F member 1 |
| | | diphosphohydrolase 3 | | | |
| 2036 | EPB41L1 | erythrocyte membrane protein | 710 | SERPING1 | serpin family G member 1 |
| | | band 4.1 like 1 | | | |
| 54566 | EPB41L4B | erythrocyte membrane protein | 26040 | SETBP1 | SET binding protein 1 |
| | | band 4.1 like 4B | | | |
| 4072 | EPCAM | epithelial cell adhesion molecule | 26470 | SEZ6L2 | seizure related 6 homolog like 2 |
| 2041 | EPHA1 | EPH receptor A1 | 2810 | SFN | stratifin |
| 1969 | EPHA2 | EPH receptor A2 | 6422 | SFRP1 | secreted frizzled related protein 1 |
| 2042 | EPHA3 | EPH receptor A3 | 6423 | SFRP2 | secreted frizzled related protein 2 |
| 285220 | EPHA6 | EPH receptor A6 | 6424 | SFRP4 | secreted frizzled related protein 4 |
| 2045 | EPHA7 | EPH receptor A7 | 6442 | SGCA | sarcoglycan alpha |
| 2048 | EPHB2 | EPH receptor B2 | 8910 | SGCE | sarcoglycan epsilon |
| 2051 | EPHB6 | EPH receptor B6 | 6446 | SGK1 | serum/glucocorticoid regulated |
| | | | | | kinase 1 |
| 2053 | EPHX2 | epoxide hydrolase 2 | 10110 | SGK2 | SGK2, serine/threonine kinase 2 |
| 79852 | EPHX3 | epoxide hydrolase 3 | 151648 | SGO1 | shugoshin 1 |
| 253152 | EPHX4 | epoxide hydrolase 4 | 130367 | SGPP2 | sphingosine-1-phosphate |
| | | | | | phosphatase 2 |
| 55040 | EPN3 | epsin 3 | 129049 | SGSM1 | small G protein signaling |
| | | | | | modulator 1 |
| 83481 | EPPK1 | epiplakin 1 | 4068 | SH2D1A | SH2 domain containing 1A |
| 54869 | EPS8L1 | EPS8 like 1 | 63898 | SH2D4A | SH2 domain containing 4A |
| 94240 | EPSTI1 | epithelial stromal interaction 1 | 6450 | SH3BGR | SH3 domain binding glutamate |
| | | | | | rich protein |
| 1833 | EPYC | epiphycan | 153769 | SH3RF2 | SH3 domain containing ring |
| | | | | | finger 2 |
| 64167 | ERAP2 | endoplasmic reticulum | 79628 | SH3TC2 | SH3 domain and |
| | | aminopeptidase 2 | | | tetratricopeptide repeats 2 |
| 2064 | ERBB2 | erb-b2 receptor tyrosine kinase 2 | 22941 | SHANK2 | SH3 and multiple ankyrin repeat |
| | | | | | domains 2 |
| 2065 | ERBB3 | erb-b2 receptor tyrosine kinase 3 | 6469 | SHH | sonic hedgehog |
| 203111 | ERICH5 | glutamate rich 5 | 134549 | SHROOM1 | shroom family member 1 |
| 79956 | ERMP1 | endoplasmic reticulum | 57619 | SHROOM3 | shroom family member 3 |
| | | metallopeptidase 1 | | | |
| 10595 | ERN2 | endoplasmic reticulum to nucleus | 54847 | SIDT1 | SID1 transmembrane family |
| | | signaling 2 | | | member 1 |
| 121506 | ERP27 | endoplasmic reticulum protein 27 | 6614 | SIGLEC1 | sialic acid binding Ig like lectin 1 |
| 54206 | ERRFI1 | ERBB receptor feedback inhibitor | 89790 | SIGLEC10 | sialic acid binding Ig like lectin 10 |
| | | 1 | | | |
| 2086 | ERV3-1 | endogenous retrovirus group 3 | 89858 | SIGLEC12 | sialic acid binding Ig like lectin 12 |
| | | member 1, envelope | | | (gene/pseudogene) |
| 100288413 | ERVMER34-1 | endogenous retrovirus group | 100049587 | SIGLEC14 | sialic acid binding Ig like lectin 14 |
| | | MER34 member 1, envelope | | | |
| 83715 | ESPN | espin | 946 | SIGLEC6 | sialic acid binding Ig like lectin 6 |
| 80004 | ESRP2 | epithelial splicing regulatory | 6493 | SIM2 | single-minded family bHLH |
| | | protein 2 | | | transcription factor 2 |
| 2118 | ETV4 | ETS variant 4 | 10326 | SIRPB1 | signal regulatory protein beta 1 |
| 59271 | EVA1C | eva-1 homolog C | 55423 | SIRPG | signal regulatory protein gamma |
| 2121 | EVC | EvC ciliary complex subunit 1 | 10736 | SIX2 | SIX homeobox 2 |
| 2123 | EVI2A | ecotropic viral integration site 2A | 221150 | SKA3 | spindle and kinetochore |
| | | | | | associated complex subunit 3 |
| 2124 | EVI2B | ecotropic viral integration site 2B | 8631 | SKAP1 | src kinase associated |
| | | | | | phosphoprotein 1 |
| 2125 | EVPL | envoplakin | 6503 | SLA | Src like adaptor |
| 645027 | EVPLL | envoplakin like | 122060 | SLAIN1 | SLAIN motif family member 1 |
| 9156 | EXO1 | exonuclease 1 | 6504 | SLAMF1 | signaling lymphocytic activation |
| | | | | | molecule family member 1 |
| 23086 | EXPH5 | exophilin 5 | 114836 | SLAMF6 | SLAM family member 6 |
| 2138 | EYA1 | EYA transcriptional coactivator | 57823 | SLAMF7 | SLAM family member 7 |
| | | and phosphatase 1 | | | |
| 2139 | EYA2 | EYA transcriptional coactivator | 56833 | SLAMF8 | SLAM family member 8 |
| | | and phosphatase 2 | | | |
| 2070 | EYA4 | EYA transcriptional coactivator | 6556 | SLC11A1 | solute carrier family 11 member 1 |
| | | and phosphatase 4 | | | |
| 2159 | F10 | coagulation factor X | 6563 | SLC14A1 | solute carrier family 14 member 1 |
| | | | | | (Kidd blood group) |
| 2162 | F13A1 | coagulation factor XIII A chain | 6564 | SLC15A1 | solute carrier family 15 member 1 |
| 2150 | F2RL1 | F2R like trypsin receptor 1 | 6566 | SLC16A1 | solute carrier family 16 member 1 |
| 2151 | F2RL2 | coagulation factor II thrombin | 117247 | SLC16A10 | solute carrier family 16 member |
| | | receptor like 2 | | | 10 |
| 2152 | F3 | coagulation factor III, tissue | 6567 | SLC16A2 | solute carrier family 16 member 2 |
| | | factor | | | |
| 2153 | F5 | coagulation factor V | 9123 | SLC16A3 | solute carrier family 16 member 3 |
| 2157 | F8 | coagulation factor VIII | 9122 | SLC16A4 | solute carrier family 16 member 4 |
| 8263 | F8A1 | coagulation factor VIII associated | 9121 | SLC16A5 | solute carrier family 16 member 5 |
| | | 1 | | | |
| 79152 | FA2H | fatty acid 2-hydroxylase | 220963 | SLC16A9 | solute carrier family 16 member 9 |
| 2166 | FAAH | fatty acid amide hydrolase | 6571 | SLC18A2 | solute carrier family 18 member |
| | | | | | A2 |
| 2170 | FABP3 | fatty acid binding protein 3 | 10560 | SLC19A2 | solute carrier family 19 member 2 |
| 2167 | FABP4 | fatty acid binding protein 4 | 6507 | SLC1A3 | solute carrier family 1 member 3 |
| 2171 | FABP5 | fatty acid binding protein 5 | 6581 | SLC22A3 | solute carrier family 22 member 3 |
| 2172 | FABP6 | fatty acid binding protein 6 | 57419 | SLC24A3 | solute carrier family 24 member 3 |
| 9415 | FADS2 | fatty acid desaturase 2 | 1468 | SLC25A10 | solute carrier family 25 member |
| | | | | | 10 |
| 374393 | FAM111B | family with sequence similarity | 9481 | SLC25A27 | solute carrier family 25 member |
| | | 111 member B | | | 27 |
| 116496 | FAM129A | family with sequence similarity | 375611 | SLC26A5 | solute carrier family 26 member 5 |
| | | 129 member A | | | |
| 728640 | FAM133CP | family with sequence similarity | 11001 | SLC27A2 | solute carrier family 27 member 2 |
| | | 133, member A pseudogene | | | |
| 220965 | FAM13C | family with sequence similarity 13 | 28965 | SLC27A6 | solute carrier family 27 member 6 |
| | | member C | | | |
| 25854 | FAM149A | family with sequence similarity | 64078 | SLC28A3 | solute carrier family 28 member 3 |
| | | 149 member A | | | |
| 728262 | FAM157A | family with sequence similarity | 3177 | SLC29A2 | solute carrier family 29 member 2 |
| | | 157 member A (non-protein | | | |
| | | coding) | | | |
| 221061 | FAM171A1 | family with sequence similarity | 55315 | SLC29A3 | solute carrier family 29 member 3 |
| | | 171 member A1 | | | |
| 400451 | FAM174B | family with sequence similarity | 6513 | SLC2A1 | solute carrier family 2 member 1 |
| | | 174 member B | | | |
| 440585 | FAM183A | family with sequence similarity | 6515 | SLC2A3 | solute carrier family 2 member 3 |
| | | 183 member A | | | |
| 79632 | FAM184A | family with sequence similarity | 6517 | SLC2A4 | solute carrier family 2 member 4 |
| | | 184 member A | | | |
| 51313 | FAM198B | family with sequence similarity | 6518 | SLC2A5 | solute carrier family 2 member 5 |
| | | 198 member B | | | |
| 54757 | FAM20A | FAM20A, golgi associated | 56606 | SLC2A9 | solute carrier family 2 member 9 |
| | | secretory pathway pseudokinase | | | |
| 56975 | FAM20C | FAM20C, golgi associated | 7780 | SLC30A2 | solute carrier family 30 member 2 |
| | | secretory pathway kinase | | | |
| 84293 | FAM213A | family with sequence similarity | 151258 | SLC38A11 | solute carrier family 38 member |
| | | 213 member A | | | 11 |
| 643161 | FAM25A | family with sequence similarity 25 | 55089 | SLC38A4 | solute carrier family 38 member 4 |
| | | member A | | | |
| 54097 | FAM3B | family with sequence similarity 3 | 92745 | SLC38A5 | solute carrier family 38 member 5 |
| | | member B | | | |
| 131177 | FAM3D | family with sequence similarity 3 | 30061 | SLC40A1 | solute carrier family 40 member 1 |
| | | member D | | | |
| 54855 | FAM46C | family with sequence similarity 46 | 126969 | SLC44A3 | solute carrier family 44 member 3 |
| | | member C | | | |
| 84985 | FAM83A | family with sequence similarity 83 | 80736 | SLC44A4 | solute carrier family 44 member 4 |
| | | member A | | | |
| 222584 | FAM83B | family with sequence similarity 83 | 204962 | SLC44A5 | solute carrier family 44 member 5 |
| | | member B | | | |
| 128876 | FAM83C | family with sequence similarity 83 | 83959 | SLC4A11 | solute carrier family 4 member 11 |
| | | member C | | | |
| 113828 | FAM83F | family with sequence similarity 83 | 6508 | SLC4A3 | solute carrier family 4 member 3 |
| | | member F | | | |
| 286077 | FAM83H | family with sequence similarity 83 | 8671 | SLC4A4 | solute carrier family 4 member 4 |
| | | member H | | | |
| 151354 | FAM84A | family with sequence similarity 84 | 113278 | SLC52A3 | solute carrier family 52 member 3 |
| | | member A | | | |
| 55138 | FAM90A1 | family with sequence similarity 90 | 6538 | SLC6A11 | solute carrier family 6 member 11 |
| | | member A1 | | | |
| 2191 | FAP | fibroblast activation protein alpha | 11254 | SLC6A14 | solute carrier family 6 member 14 |
| 55711 | FAR2 | fatty acyl-CoA reductase 2 | 28968 | SLC6A16 | solute carrier family 6 member 16 |
| 2194 | FASN | fatty acid synthase | 6535 | SLC6A8 | solute carrier family 6 member 8 |
| 2196 | FAT2 | FAT atypical cadherin 2 | 6542 | SLC7A2 | solute carrier family 7 member 2 |
| 120114 | FAT3 | FAT atypical cadherin 3 | 8140 | SLC7A5 | solute carrier family 7 member 5 |
| 79633 | FAT4 | FAT atypical cadherin 4 | 9056 | SLC7A7 | solute carrier family 7 member 7 |
| 2192 | FBLN1 | fibulin 1 | 6546 | SLC8A1 | solute carrier family 8 member |
| | | | | | A1 |
| 2199 | FBLN2 | fibulin 2 | 6549 | SLC9A2 | solute carrier family 9 member |
| | | | | | A2 |
| 10516 | FBLN5 | fibulin 5 | 389015 | SLC9A4 | solute carrier family 9 member |
| | | | | | A4 |
| 2200 | FBN1 | fibrillin 1 | 28231 | SLCO4A1 | solute carrier organic anion |
| | | | | | transporter family member 4A1 |
| 2201 | FBN2 | fibrillin 2 | 100506736 | SLFN12L | schlafen family member 12 like |
| 2203 | FBP1 | fructose-bisphosphatase 1 | 146857 | SLFN13 | schlafen family member 13 |
| 126433 | FBXO27 | F-box protein 27 | 9353 | SLIT2 | slit guidance ligand 2 |
| 114907 | FBXO32 | F-box protein 32 | 6586 | SLIT3 | slit guidance ligand 3 |
| 2204 | FCAR | Fc fragment of IgA receptor | 84189 | SLITRK6 | SLIT and NTRK like family |
| | | | | | member 6 |
| 2205 | FCER1A | Fc fragment of IgE receptor Ia | 6590 | SLPI | secretory leukocyte peptidase |
| | | | | | inhibitor |
| 2207 | FCER1G | Fc fragment of IgE receptor Ig | 4091 | SMAD6 | SMAD family member 6 |
| 8857 | FCGBP | Fc fragment of IgG binding | 4093 | SMAD9 | SMAD family member 9 |
| | | protein | | | |
| 2209 | FCGR1A | Fc fragment of IgG receptor Ia | 6604 | SMARCD3 | SWI/SNF related, matrix |
| | | | | | associated, actin dependent |
| | | | | | regulator of chromatin, subfamily |
| | | | | | d, member 3 |
| 2212 | FCGR2A | Fc fragment of IgG receptor IIa | 27127 | SMC1B | structural maintenance of |
| | | | | | chromosomes 1B |
| 2213 | FCGR2B | Fc fragment of IgG receptor IIb | 595101 | SMG1P5 | SMG1 pseudogene 5 |
| 9103 | FCGR2C | Fc fragment of IgG receptor IIc | 440335 | SMIM22 | small integral membrane protein |
| | | (gene/pseudogene) | | | 22 |
| 2214 | FCGR3A | Fc fragment of IgG receptor IIIa | 64093 | SMOC1 | SPARC related modular calcium |
| | | | | | binding 1 |
| 2215 | FCGR3B | Fc fragment of IgG receptor IIIb | 64094 | SMOC2 | SPARC related modular calcium |
| | | | | | binding 2 |
| 9214 | FCMR | Fc fragment of IgM receptor | 27293 | SMPDL3B | sphingomyelin |
| | | | | | phosphodiesterase acid like 3B |
| 2219 | FCN1 | ficolin 1 | 23676 | SMPX | small muscle protein, X-linked |
| 115350 | FCRL1 | Fc receptor like 1 | 6525 | SMTN | smoothelin |
| 79368 | FCRL2 | Fc receptor like 2 | 6591 | SNAI2 | snail family transcriptional |
| | | | | | repressor 2 |
| 115352 | FCRL3 | Fc receptor like 3 | 6622 | SNCA | synuclein alpha |
| 83416 | FCRL5 | Fc receptor like 5 | 9627 | SNCAIP | synuclein alpha interacting |
| | | | | | protein |
| 260436 | FDCSP | follicular dendritic cell secreted | 6623 | SNCG | synuclein gamma |
| | | protein | | | |
| 80307 | FER1L4 | fer-1 like family member 4, | 25992 | SNED1 | sushi, nidogen and EGF like |
| | | pseudogene | | | domains 1 |
| 55612 | FERMT1 | fermitin family member 1 | 677792 | SNORA1 | small nucleolar RNA, H/ACA box |
| | | | | | 1 |
| 10979 | FERMT2 | fermitin family member 2 | 574042 | SNORA10 | small nucleolar RNA, H/ACA box |
| | | | | | 10 |
| 89846 | FGD3 | FYVE, RhoGEF and PH domain | 677799 | SNORA11 | small nucleolar RNA, H/ACA box |
| | | containing 3 | | | 11 |
| 2255 | FGF10 | fibroblast growth factor 10 | 100124539 | SNORA11B | small nucleolar RNA, H/ACA box |
| | | | | | 11B |
| 2247 | FGF2 | fibroblast growth factor 2 | 654322 | SNORA13 | small nucleolar RNA, H/ACA box |
| | | | | | 13 |
| 2252 | FGF7 | fibroblast growth factor 7 | 677801 | SNORA14A | small nucleolar RNA, H/ACA box |
| | | | | | 14A |
| 9982 | FGFBP1 | fibroblast growth factor binding | 677802 | SNORA14B | small nucleolar RNA, H/ACA box |
| | | protein 1 | | | 14B |
| 2260 | FGFR1 | fibroblast growth factor receptor | 677803 | SNORA15 | small nucleolar RNA, H/ACA box |
| | | 1 | | | 15 |
| 2263 | FGFR2 | fibroblast growth factor receptor | 692073 | SNORA16A | small nucleolar RNA, H/ACA box |
| | | 2 | | | 16A |
| 2261 | FGFR3 | fibroblast growth factor receptor | 692157 | SNORA16B | small nucleolar RNA, H/ACA box |
| | | 3 | | | 16B |
| 10875 | FGL2 | fibrinogen like 2 | 677805 | SNORA18 | small nucleolar RNA, H/ACA box |
| | | | | | 18 |
| 114827 | FHAD1 | forkhead associated | 641451 | SNORA19 | small nucleolar RNA, H/ACA box |
| | | phosphopeptide binding domain | | | 19 |
| | | 1 | | | |
| 2273 | FHL1 | four and a half LIM domains 1 | 677806 | SNORA20 | small nucleolar RNA, H/ACA box |
| | | | | | 20 |
| 80206 | FHOD3 | formin homology 2 domain | 619505 | SNORA21 | small nucleolar RNA, H/ACA box |
| | | containing 3 | | | 21 |
| 387758 | FIBIN | fin bud initiation factor homolog | 677809 | SNORA24 | small nucleolar RNA, H/ACA box |
| | | (zebrafish) | | | 24 |
| 27145 | FILIP1 | filamin A interacting protein 1 | 684959 | SNORA25 | small nucleolar RNA, H/ACA box |
| | | | | | 25 |
| 11259 | FILIP1L | filamin A interacting protein 1 like | 677810 | SNORA26 | small nucleolar RNA, H/ACA box |
| | | | | | 26 |
| 24147 | FJX1 | four jointed box 1 | 619499 | SNORA27 | small nucleolar RNA, H/ACA box |
| | | | | | 27 |
| 60681 | FKBP10 | FK506 binding protein 10 | 677811 | SNORA28 | small nucleolar RNA, H/ACA box |
| | | | | | 28 |
| 2289 | FKBP5 | FK506 binding protein 5 | 677812 | SNORA29 | small nucleolar RNA, H/ACA box |
| | | | | | 29 |
| 339400 | FLG-AS1 | FLG antisense RNA 1 | 677793 | SNORA2A | small nucleolar RNA, H/ACA box |
| | | | | | 2A |
| 200058 | FLJ23867 | uncharacterized protein | 677794 | SNORA2B | small nucleolar RNA, H/ACA box |
| | | FLJ23867 | | | 2B |
| 2316 | FLNA | filamin A | 677813 | SNORA30 | small nucleolar RNA, H/ACA box |
| | | | | | 30 |
| 2318 | FLNC | filamin C | 677814 | SNORA31 | small nucleolar RNA, H/ACA box |
| | | | | | 31 |
| 23767 | FLRT3 | fibronectin leucine rich | 692063 | SNORA32 | small nucleolar RNA, H/ACA box |
| | | transmembrane protein 3 | | | 32 |
| 2322 | FLT3 | fms related tyrosine kinase 3 | 594839 | SNORA33 | small nucleolar RNA, H/ACA box |
| | | | | | 33 |
| 2326 | FMO1 | flavin containing monooxygenase | 677818 | SNORA36B | small nucleolar RNA, H/ACA box |
| | | 1 | | | 36B |
| 2327 | FMO2 | flavin containing monooxygenase | 677820 | SNORA38 | small nucleolar RNA, H/ACA box |
| | | 2 | | | 38 |
| 2328 | FMO3 | flavin containing monooxygenase | 100124536 | SNORA38B | small nucleolar RNA, H/ACA box |
| | | 3 | | | 38B |
| 2330 | FMO5 | flavin containing monooxygenase | 619562 | SNORA3A | small nucleolar RNA, H/ACA box |
| | | 5 | | | 3A |
| 116123 | FMO9P | flavin containing monooxygenase | 677826 | SNORA3B | small nucleolar RNA, H/ACA box |
| | | 9 pseudogene | | | 3B |
| 2331 | FMOD | fibromodulin | 619568 | SNORA4 | small nucleolar RNA, H/ACA box |
| | | | | | 4 |
| 2335 | FN1 | fibronectin 1 | 677822 | SNORA40 | small nucleolar RNA, H/ACA box |
| | | | | | 40 |
| 84624 | FNDC1 | fibronectin type III domain | 619569 | SNORA41 | small nucleolar RNA, H/ACA box |
| | | containing 1 | | | 41 |
| 2350 | FOLR2 | folate receptor beta | 677825 | SNORA44 | small nucleolar RNA, H/ACA box |
| | | | | | 44 |
| 2353 | FOS | Fos proto-oncogene, AP-1 | 677827 | SNORA46 | small nucleolar RNA, H/ACA box |
| | | transcription factor subunit | | | 46 |
| 2354 | FOSB | FosB proto-oncogene, AP-1 | 677828 | SNORA47 | small nucleolar RNA, H/ACA box |
| | | transcription factor subunit | | | 47 |
| 8061 | FOSL1 | FOS like 1, AP-1 transcription | 677830 | SNORA50A | small nucleolar RNA, H/ACA box |
| | | factor subunit | | | 50A |
| 3169 | FOXA1 | forkhead box A1 | 677842 | SNORA50C | small nucleolar RNA, H/ACA box |
| | | | | | 50C |
| 2296 | FOXC1 | forkhead box C1 | 677831 | SNORA51 | small nucleolar RNA, H/ACA box |
| | | | | | 51 |
| 2303 | FOXC2 | forkhead box C2 | 677833 | SNORA54 | small nucleolar RNA, H/ACA box |
| | | | | | 54 |
| 2297 | FOXD1 | forkhead box D1 | 677834 | SNORA55 | small nucleolar RNA, H/ACA box |
| | | | | | 55 |
| 2294 | FOXF1 | forkhead box F1 | 677836 | SNORA58 | small nucleolar RNA, H/ACA box |
| | | | | | 58 |
| 2295 | FOXF2 | forkhead box F2 | 677796 | SNORA5C | small nucleolar RNA, H/ACA box |
| | | | | | 5C |
| 2300 | FOXL1 | forkhead box L1 | 574040 | SNORA6 | small nucleolar RNA, H/ACA box |
| | | | | | 6 |
| 2305 | FOXM1 | forkhead box M1 | 677837 | SNORA60 | small nucleolar RNA, H/ACA box |
| | | | | | 60 |
| 8456 | FOXN1 | forkhead box N1 | 677838 | SNORA61 | small nucleolar RNA, H/ACA box |
| | | | | | 61 |
| 100132074 | FOXO6 | forkhead box O6 | 6044 | SNORA62 | small nucleolar RNA, H/ACA box |
| | | | | | 62 |
| 93986 | FOXP2 | forkhead box P2 | 26783 | SNORA65 | small nucleolar RNA, H/ACA box |
| | | | | | 65 |
| 94234 | FOXQ1 | forkhead box Q1 | 26782 | SNORA66 | small nucleolar RNA, H/ACA box |
| | | | | | 66 |
| 2357 | FPR1 | formyl peptide receptor 1 | 26781 | SNORA67 | small nucleolar RNA, H/ACA box |
| | | | | | 67 |
| 2358 | FPR2 | formyl peptide receptor 2 | 26779 | SNORA69 | small nucleolar RNA, H/ACA box |
| | | | | | 69 |
| 2359 | FPR3 | formyl peptide receptor 3 | 26777 | SNORA71A | small nucleolar RNA, H/ACA box |
| | | | | | 71A |
| 80144 | FRAS1 | Fraser extracellular matrix | 26776 | SNORA71B | small nucleolar RNA, H/ACA box |
| | | complex subunit 1 | | | 71B |
| 158326 | FREM1 | FRAS1 related extracellular | 677839 | SNORA71C | small nucleolar RNA, H/ACA box |
| | | matrix 1 | | | 71C |
| 341640 | FREM2 | FRAS1 related extracellular | 677840 | SNORA71D | small nucleolar RNA, H/ACA box |
| | | matrix protein 2 | | | 71D |
| 642236 | FRG1JP | FSHD region gene 1 family | 677821 | SNORA71E | small nucleolar RNA, H/ACA box |
| | | member J, pseudogene | | | 71E |
| 391059 | FRRS1 | ferric chelate reductase 1 | 26775 | SNORA72 | small nucleolar RNA, H/ACA box |
| | | | | | 72 |
| 10129 | FRY | FRY microtubule binding protein | 654321 | SNORA75 | small nucleolar RNA, H/ACA box |
| | | | | | 75 |
| 2487 | FRZB | frizzled related protein | 677843 | SNORA77 | small nucleolar RNA, H/ACA box |
| | | | | | 77 |
| 6624 | FSCN1 | fascin actin-bundling protein 1 | 677844 | SNORA78 | small nucleolar RNA, H/ACA box |
| | | | | | 78 |
| 401024 | FSIP2 | fibrous sheath interacting protein | 677845 | SNORA79 | small nucleolar RNA, H/ACA box |
| | | 2 | | | 79 |
| 10468 | FST | follistatin | 677797 | SNORA7B | small nucleolar RNA, H/ACA box |
| | | | | | 7B |
| 10272 | FSTL3 | follistatin like 3 | 677846 | SNORA80A | small nucleolar RNA, H/ACA box |
| | | | | | 80A |
| 23105 | FSTL4 | follistatin like 4 | 100302743 | SNORA80B | small nucleolar RNA, H/ACA box |
| | | | | | 80B |
| 2524 | FUT2 | fucosyltransferase 2 | 677823 | SNORA80E | small nucleolar RNA, H/ACA box |
| | | | | | 80E |
| 2525 | FUT3 | fucosyltransferase 3 (Lewis blood | 677798 | SNORA9 | small nucleolar RNA, H/ACA box |
| | | group) | | | 9 |
| 2528 | FUT6 | fucosyltransferase 6 | 594838 | SNORD100 | small nucleolar RNA, C/D box |
| | | | | | 100 |
| 5348 | FXYD1 | FXYD domain containing ion | 692200 | SNORD103C | small nucleolar RNA, C/D box |
| | | transport regulator 1 | | | 103C |
| 5349 | FXYD3 | FXYD domain containing ion | 692227 | SNORD104 | small nucleolar RNA, C/D box |
| | | transport regulator 3 | | | 104 |
| 53828 | FXYD4 | FXYD domain containing ion | 692229 | SNORD105 | small nucleolar RNA, C/D box |
| | | transport regulator 4 | | | 105 |
| 53826 | FXYD6 | FXYD domain containing ion | 692058 | SNORD11 | small nucleolar RNA, C/D box 11 |
| | | transport regulator 6 | | | |
| 2533 | FYB1 | FYN binding protein 1 | 692213 | SNORD110 | small nucleolar RNA, C/D box |
| | | | | | 110 |
| 50486 | GOS2 | G0/G1 switch 2 | 767592 | SNORD114-14 | small nucleolar RNA, C/D box |
| | | | | | 114-14 |
| 9568 | GABBR2 | gamma-aminobutyric acid type B | 100033807 | SNORD115-33 | small nucleolar RNA, C/D box |
| | | receptor subunit 2 | | | 115-33 |
| 2556 | GABRA3 | gamma-aminobutyric acid type A | 100033413 | SNORD116-1 | small nucleolar RNA, C/D box |
| | | receptor alpha3 subunit | | | 116-1 |
| 2562 | GABRB3 | gamma-aminobutyric acid type A | 100033426 | SNORD116-14 | small nucleolar RNA, C/D box |
| | | receptor beta3 subunit | | | 116-14 |
| 2564 | GABRE | gamma-aminobutyric acid type A | 100033427 | SNORD116-15 | small nucleolar RNA, C/D box |
| | | receptor epsilon subunit | | | 116-15 |
| 2568 | GABRP | gamma-aminobutyric acid type A | 100033428 | SNORD116-16 | small nucleolar RNA, C/D box |
| | | receptor pi subunit | | | 116-16 |
| 55879 | GABRQ | gamma-aminobutyric acid type A | 100033414 | SNORD116-2 | small nucleolar RNA, C/D box |
| | | receptor theta subunit | | | 116-2 |
| 2571 | GAD1 | glutamate decarboxylase 1 | 100033431 | SNORD116-20 | small nucleolar RNA, C/D box |
| | | | | | 116-20 |
| 4616 | GADD45B | growth arrest and DNA damage | 100033432 | SNORD116-21 | small nucleolar RNA, C/D box |
| | | inducible beta | | | 116-21 |
| 51083 | GAL | galanin and GMAP prepropeptide | 100033433 | SNORD116-22 | small nucleolar RNA, C/D box |
| | | | | | 116-22 |
| 2589 | GALNT1 | polypeptide N- | 100033434 | SNORD116-23 | small nucleolar RNA, C/D box |
| | | acetylgalactosaminyltransferase | | | 116-23 |
| | | 1 | | | |
| 79695 | GALNT12 | polypeptide N- | 100033435 | SNORD116-24 | small nucleolar RNA, C/D box |
| | | acetylgalactosaminyltransferase | | | 116-24 |
| | | 12 | | | |
| 79623 | GALNT14 | polypeptide N- | 100033436 | SNORD116-25 | small nucleolar RNA, C/D box |
| | | acetylgalactosaminyltransferase | | | 116-25 |
| | | 14 | | | |
| 57452 | GALNT16 | polypeptide N- | 100033438 | SNORD116-26 | small nucleolar RNA, C/D box |
| | | acetylgalactosaminyltransferase | | | 116-26 |
| | | 16 | | | |
| 64409 | GALNT17 | polypeptide N- | 100033439 | SNORD116-27 | small nucleolar RNA, C/D box |
| | | acetylgalactosaminyltransferase | | | 116-27 |
| | | 17 | | | |
| 11227 | GALNT5 | polypeptide N- | 100033821 | SNORD116-29 | small nucleolar RNA, C/D box |
| | | acetylgalactosaminyltransferase | | | 116-29 |
| | | 5 | | | |
| 54433 | GAR1 | GAR1 ribonucleoprotein | 100033418 | SNORD116-6 | small nucleolar RNA, C/D box |
| | | | | | 116-6 |
| 64762 | GAREM1 | GRB2 associated regulator of | 100033420 | SNORD116-8 | small nucleolar RNA, C/D box |
| | | MAPK1 subtype 1 | | | 116-8 |
| 2619 | GAS1 | growth arrest specific 1 | 692233 | SNORD117 | small nucleolar RNA, C/D box |
| | | | | | 117 |
| 2621 | GAS6 | growth arrest specific 6 | 692057 | SNORD12 | small nucleolar RNA, C/D box 12 |
| 8522 | GAS7 | growth arrest specific 7 | 100113393 | SNORD12B | small nucleolar RNA, C/D box |
| | | | | | 12B |
| 2624 | GATA2 | GATA binding protein 2 | 26765 | SNORD12C | small nucleolar RNA, C/D box |
| | | | | | 12C |
| 2625 | GATA3 | GATA binding protein 3 | 692084 | SNORD13 | small nucleolar RNA, C/D box 13 |
| 2627 | GATA6 | GATA binding protein 6 | 26822 | SNORD14A | small nucleolar RNA, C/D box |
| | | | | | 14A |
| 2628 | GATM | glycine amidinotransferase | 85388 | SNORD14B | small nucleolar RNA, C/D box |
| | | | | | 14B |
| 2633 | GBP1 | guanylate binding protein 1 | 6079 | SNORD15A | small nucleolar RNA, C/D box |
| | | | | | 15A |
| 400759 | GBP1P1 | guanylate binding protein 1 | 114599 | SNORD15B | small nucleolar RNA, C/D box |
| | | pseudogene 1 | | | 15B |
| 2635 | GBP3 | guanylate binding protein 3 | 595097 | SNORD16 | small nucleolar RNA, C/D box 16 |
| 115361 | GBP4 | guanylate binding protein 4 | 677850 | SNORD1C | small nucleolar RNA, C/D box 1C |
| 115362 | GBP5 | guanylate binding protein 5 | 6083 | SNORD21 | small nucleolar RNA, C/D box 21 |
| 163351 | GBP6 | guanylate binding protein family | 9304 | SNORD22 | small nucleolar RNA, C/D box 22 |
| | | member 6 | | | |
| 2729 | GCLC | glutamate-cysteine ligase | 26820 | SNORD24 | small nucleolar RNA, C/D box 24 |
| | | catalytic subunit | | | |
| 2650 | GCNT1 | glucosaminyl (N-acetyl) | 9302 | SNORD26 | small nucleolar RNA, C/D box 26 |
| | | transferase 1, core 2 | | | |
| 2651 | GCNT2 | glucosaminyl (N-acetyl) | 9301 | SNORD27 | small nucleolar RNA, C/D box 27 |
| | | transferase 2, I-branching | | | |
| | | enzyme (I blood group) | | | |
| 51301 | GCNT4 | glucosaminyl (N-acetyl) | 9300 | SNORD28 | small nucleolar RNA, C/D box 28 |
| | | transferase 4, core 2 | | | |
| 9615 | GDA | guanine deaminase | 9297 | SNORD29 | small nucleolar RNA, C/D box 29 |
| 9518 | GDF15 | growth differentiation factor 15 | 9298 | SNORD31 | small nucleolar RNA, C/D box 31 |
| 151449 | GDF7 | growth differentiation factor 7 | 26819 | SNORD32A | small nucleolar RNA, C/D box |
| | | | | | 32A |
| 54857 | GDPD2 | glycerophosphodiester | 26818 | SNORD33 | small nucleolar RNA, C/D box 33 |
| | | phosphodiesterase domain | | | |
| | | containing 2 | | | |
| 79153 | GDPD3 | glycerophosphodiester | 26817 | SNORD34 | small nucleolar RNA, C/D box 34 |
| | | phosphodiesterase domain | | | |
| | | containing 3 | | | |
| 2669 | GEM | GTP binding protein | 26816 | SNORD35A | small nucleolar RNA, C/D box |
| | | overexpressed in skeletal muscle | | | 35A |
| 9945 | GFPT2 | glutamine-fructose-6-phosphate | 84546 | SNORD35B | small nucleolar RNA, C/D box |
| | | transaminase 2 | | | 35B |
| 2676 | GFRA3 | GDNF family receptor alpha 3 | 26815 | SNORD36A | small nucleolar RNA, C/D box |
| | | | | | 36A |
| 8836 | GGH | gamma-glutamyl hydrolase | 26813 | SNORD36C | small nucleolar RNA, C/D box |
| | | | | | 36C |
| 2687 | GGT5 | gamma-glutamyltransferase 5 | 94162 | SNORD38A | small nucleolar RNA, C/D box |
| | | | | | 38A |
| 124975 | GGT6 | gamma-glutamyltransferase 6 | 94163 | SNORD38B | small nucleolar RNA, C/D box |
| | | | | | 38B |
| 2681 | GGTA1P | glycoprotein, alpha- | 780851 | SNORD3A | small nucleolar RNA, C/D box 3A |
| | | galactosyltransferase 1 | | | |
| | | pseudogene | | | |
| 2690 | GHR | growth hormone receptor | 26851 | SNORD3B-1 | small nucleolar RNA, C/D box |
| | | | | | 3B-1 |
| 55303 | GIMAP4 | GTPase, IMAP family member 4 | 780852 | SNORD3B-2 | small nucleolar RNA, C/D box |
| | | | | | 3B-2 |
| 168537 | GIMAP7 | GTPase, IMAP family member 7 | 780853 | SNORD3C | small nucleolar RNA, C/D box 3C |
| 51659 | GINS2 | GINS complex subunit 2 | 780854 | SNORD3D | small nucleolar RNA, C/D box 3D |
| 54810 | GIPC2 | GIPC PDZ domain containing | 26810 | SNORD41 | small nucleolar RNA, C/D box 41 |
| | | family member 2 | | | |
| 2697 | GJA1 | gap junction protein alpha 1 | 26805 | SNORD45A | small nucleolar RNA, C/D box |
| | | | | | 45A |
| 2702 | GJA5 | gap junction protein alpha 5 | 692085 | SNORD45C | small nucleolar RNA, C/D box |
| | | | | | 45C |
| 2706 | GJB2 | gap junction protein beta 2 | 94161 | SNORD46 | small nucleolar RNA, C/D box 46 |
| 2707 | GJB3 | gap junction protein beta 3 | 26802 | SNORD47 | small nucleolar RNA, C/D box 47 |
| 127534 | GJB4 | gap junction protein beta 4 | 26801 | SNORD48 | small nucleolar RNA, C/D box 48 |
| 2709 | GJB5 | gap junction protein beta 5 | 26773 | SNORD4A | small nucleolar RNA, C/D box 4A |
| 10804 | GJB6 | gap junction protein beta 6 | 26799 | SNORD50A | small nucleolar RNA, C/D box |
| | | | | | 50A |
| 375519 | GJB7 | gap junction protein beta 7 | 26796 | SNORD53 | small nucleolar RNA, C/D box 53 |
| 89944 | GLB1L2 | galactosidase beta 1 like 2 | 26811 | SNORD55 | small nucleolar RNA, C/D box 55 |
| 2731 | GLDC | glycine decarboxylase | 26793 | SNORD56 | small nucleolar RNA, C/D box 56 |
| 2735 | GLI1 | GLI family zinc finger 1 | 26792 | SNORD57 | small nucleolar RNA, C/D box 57 |
| 2736 | GLI2 | GLI family zinc finger 2 | 26791 | SNORD58A | small nucleolar RNA, C/D box |
| | | | | | 58A |
| 2737 | GLI3 | GLI family zinc finger 3 | 26788 | SNORD60 | small nucleolar RNA, C/D box 60 |
| 84662 | GLIS2 | GLIS family zinc finger 2 | 26787 | SNORD61 | small nucleolar RNA, C/D box 61 |
| 169792 | GLIS3 | GLIS family zinc finger 3 | 26785 | SNORD63 | small nucleolar RNA, C/D box 63 |
| 2743 | GLRB | glycine receptor beta | 692106 | SNORD65 | small nucleolar RNA, C/D box 65 |
| 83468 | GLT8D2 | glycosyltransferase 8 domain | 692107 | SNORD66 | small nucleolar RNA, C/D box 66 |
| | | containing 2 | | | |
| 9630 | GNA14 | G protein subunit alpha 14 | 692108 | SNORD67 | small nucleolar RNA, C/D box 67 |
| 2774 | GNAL | G protein subunit alpha L | 606500 | SNORD68 | small nucleolar RNA, C/D box 68 |
| 10578 | GNLY | granulysin | 692109 | SNORD69 | small nucleolar RNA, C/D box 69 |
| 440270 | GOLGA8B | golgin A8 family member B | 692111 | SNORD71 | small nucleolar RNA, C/D box 71 |
| 127845 | GOLT1A | golgi transport 1A | 619498 | SNORD74 | small nucleolar RNA, C/D box 74 |
| 2811 | GP1BA | glycoprotein Ib platelet alpha | 692196 | SNORD76 | small nucleolar RNA, C/D box 76 |
| | | subunit | | | |
| 51206 | GP6 | glycoprotein VI platelet | 26774 | SNORD80 | small nucleolar RNA, C/D box 80 |
| 2719 | GPC3 | glypican 3 | 25826 | SNORD82 | small nucleolar RNA, C/D box 82 |
| 2239 | GPC4 | glypican 4 | 116937 | SNORD83A | small nucleolar RNA, C/D box |
| | | | | | 83A |
| 10082 | GPC6 | glypican 6 | 116938 | SNORD83B | small nucleolar RNA, C/D box |
| | | | | | 83B |
| 23171 | GPD1L | glycerol-3-phosphate | 692199 | SNORD84 | small nucleolar RNA, C/D box 84 |
| | | dehydrogenase 1 like | | | |
| 10457 | GPNMB | glycoprotein nmb | 641648 | SNORD87 | small nucleolar RNA, C/D box 87 |
| 4935 | GPR143 | G protein-coupled receptor 143 | 692204 | SNORD88C | small nucleolar RNA, C/D box |
| | | | | | 88C |
| 2838 | GPR15 | G protein-coupled receptor 15 | 692205 | SNORD89 | small nucleolar RNA, C/D box 89 |
| 26996 | GPR160 | G protein-coupled receptor 160 | 692053 | SNORD9 | small nucleolar RNA, C/D box 9 |
| 29909 | GPR171 | G protein-coupled receptor 171 | 692206 | SNORD90 | small nucleolar RNA, C/D box 90 |
| 84636 | GPR174 | G protein-coupled receptor 174 | 692208 | SNORD91B | small nucleolar RNA, C/D box |
| | | | | | 91B |
| 1880 | GPR183 | G protein-coupled receptor 183 | 692209 | SNORD92 | small nucleolar RNA, C/D box 92 |
| 2863 | GPR39 | G protein-coupled receptor 39 | 692225 | SNORD94 | small nucleolar RNA, C/D box 94 |
| 53836 | GPR87 | G protein-coupled receptor 87 | 619570 | SNORD95 | small nucleolar RNA, C/D box 95 |
| 9052 | GPRC5A | G protein-coupled receptor class | 619571 | SNORD96A | small nucleolar RNA, C/D box |
| | | C group 5 member A | | | 96A |
| 55890 | GPRC5C | G protein-coupled receptor class | 692223 | SNORD97 | small nucleolar RNA, C/D box 97 |
| | | C group 5 member C | | | |
| 84706 | GPT2 | glutamic -- pyruvic transaminase 2 | 692212 | SNORD99 | small nucleolar RNA, C/D box 99 |
| 2877 | GPX2 | glutathione peroxidase 2 | 29887 | SNX10 | sorting nexin 10 |
| 2878 | GPX3 | glutathione peroxidase 3 | 100652781 | SNX29P1 | sorting nexin 29 pseudogene 1 |
| 493869 | GPX8 | glutathione peroxidase 8 | 440352 | SNX29P2 | sorting nexin 29 pseudogene 2 |
| | | (putative) | | | |
| 2888 | GRB14 | growth factor receptor bound | 169166 | SNX31 | sorting nexin 31 |
| | | protein 14 | | | |
| 2886 | GRB7 | growth factor receptor bound | 55084 | SOBP | sine oculis binding protein |
| | | protein 7 | | | homolog |
| 9687 | GREB1 | growth regulation by estrogen in | 8835 | SOCS2 | suppressor of cytokine signaling |
| | | breast cancer 1 | | | 2 |
| 80000 | GREB1L | growth regulation by estrogen in | 9021 | SOCS3 | suppressor of cytokine signaling |
| | | breast cancer 1 like | | | 3 |
| 26585 | GREM1 | gremlin 1, DAN family BMP | 6648 | SOD2 | superoxide dismutase 2 |
| | | antagonist | | | |
| 29841 | GRHL1 | grainyhead like transcription | 6649 | SOD3 | superoxide dismutase 3 |
| | | factor 1 | | | |
| 79977 | GRHL2 | grainyhead like transcription | 10580 | SORBS1 | sorbin and SH3 domain |
| | | factor 2 | | | containing 1 |
| 57822 | GRHL3 | grainyhead like transcription | 8470 | SORBS2 | sorbin and SH3 domain |
| | | factor 3 | | | containing 2 |
| 2898 | GRIK2 | glutamate ionotropic receptor | 57537 | SORCS2 | sortilin related VPS10 domain |
| | | kainate type subunit 2 | | | containing receptor 2 |
| 2906 | GRIN2D | glutamate ionotropic receptor | 6652 | SORD | sorbitol dehydrogenase |
| | | NMDA type subunit 2D | | | |
| 56169 | GSDMC | gasdermin C | 6653 | SORL1 | sortilin related receptor 1 |
| 1687 | GSDME | gasdermin E | 345079 | SOWAHB | sosondowah ankyrin repeat |
| | | | | | domain family member B |
| 2938 | GSTA1 | glutathione S-transferase alpha 1 | 6657 | SOX2 | SRY-box 2 |
| 2941 | GSTA4 | glutathione S-transferase alpha 4 | 6662 | SOX9 | SRY-box 9 |
| 2944 | GSTM1 | glutathione S-transferase mu 1 | 11262 | SP140 | SP140 nuclear body protein |
| 2946 | GSTM2 | glutathione S-transferase mu 2 | 200162 | SPAG17 | sperm associated antigen 17 |
| 442245 | GSTM2P1 | glutathione S-transferase mu 2 | 6676 | SPAG4 | sperm associated antigen 4 |
| | | pseudogene 1 | | | |
| 2947 | GSTM3 | glutathione S-transferase mu 3 | 6678 | SPARC | secreted protein acidic and |
| | | | | | cysteine rich |
| 2948 | GSTM4 | glutathione S-transferase mu 4 | 8404 | SPARCL1 | SPARC like 1 |
| 2949 | GSTM5 | glutathione S-transferase mu 5 | 128153 | SPATA17 | spermatogenesis associated 17 |
| 653689 | GSTT2B | glutathione S-transferase theta | 57405 | SPC25 | SPC25, NDC80 kinetochore |
| | | 2B (gene/pseudogene) | | | complex component |
| 441124 | GTF2IP20 | general transcription factor IIi | 10290 | SPEG | SPEG complex locus |
| | | pseudogene 20 | | | |
| 121355 | GTSF1 | gametocyte specific factor 1 | 246777 | SPESP1 | sperm equatorial segment |
| | | | | | protein 1 |
| 51454 | GULP1 | GULP, engulfment adaptor PTB | 6688 | SPI1 | Spi-1 proto-oncogene |
| | | domain containing 1 | | | |
| 387751 | GVINP1 | GTPase, very large interferon | 6690 | SPINK1 | serine peptidase inhibitor, Kazal |
| | | inducible pseudogene 1 | | | type 1 |
| 727936 | GXYLT2 | glucoside xylosyltransferase 2 | 11005 | SPINK5 | serine peptidase inhibitor, Kazal |
| | | | | | type 5 |
| 2995 | GYPC | glycophorin C (Gerbich blood | 6692 | SPINT1 | serine peptidase inhibitor, Kunitz |
| | | group) | | | type 1 |
| 3001 | GZMA | granzyme A | 10653 | SPINT2 | serine peptidase inhibitor, Kunitz |
| | | | | | type 2 |
| 3002 | GZMB | granzyme B | 84501 | SPIRE2 | spire type actin nucleation factor |
| | | | | | 2 |
| 2999 | GZMH | granzyme H | 124976 | SPNS2 | sphingolipid transporter 2 |
| 3003 | GZMK | granzyme K | 90853 | SPOCD1 | SPOC domain containing 1 |
| 283120 | H19 | H19, imprinted maternally | 6695 | SPOCK1 | SPARC/osteonectin, cwcv and |
| | | expressed transcript (non-protein | | | kazal like domains proteoglycan |
| | | coding) | | | 1 |
| 23498 | HAAO | 3-hydroxyanthranilate 3,4- | 9806 | SPOCK2 | SPARC/osteonectin, cwcv and |
| | | dioxygenase | | | kazal like domains proteoglycan |
| | | | | | 2 |
| 9200 | HACD1 | 3-hydroxyacyl-CoA dehydratase | 50859 | SPOCK3 | SPARC/osteonectin, cwcv and |
| | | 1 | | | kazal like domains proteoglycan |
| | | | | | 3 |
| 57817 | HAMP | hepcidin antimicrobial peptide | 10418 | SPON1 | spondin 1 |
| 9464 | HAND2 | heart and neural crest derivatives | 1041 | SPON2 | spondin 2 |
| | | expressed 2 | | | |
| 145864 | HAPLN3 | hyaluronan and proteoglycan link | 6696 | SPP1 | secreted phosphoprotein 1 |
| | | protein 3 | | | |
| 3037 | HAS2 | hyaluronan synthase 2 | 6698 | SPRR1A | small proline rich protein 1A |
| 3038 | HAS3 | hyaluronan synthase 3 | 6699 | SPRR1B | small proline rich protein 1B |
| 26762 | HAVCR1 | hepatitis A virus cellular receptor | 6707 | SPRR3 | small proline rich protein 3 |
| | | 1 | | | |
| 84868 | HAVCR2 | hepatitis A virus cellular receptor | 10252 | SPRY1 | sprouty RTK signaling antagonist |
| | | 2 | | | 1 |
| 3039 | HBA1 | hemoglobin subunit alpha 1 | 6712 | SPTBN2 | spectrin beta, non-erythrocytic 2 |
| 3040 | HBA2 | hemoglobin subunit alpha 2 | 55304 | SPTLC3 | serine palmitoyltransferase long |
| | | | | | chain base subunit 3 |
| 3043 | HBB | hemoglobin subunit beta | 165679 | SPTSSB | serine palmitoyltransferase small |
| | | | | | subunit B |
| 1839 | HBEGF | heparin binding EGF like growth | 5552 | SRGN | serglycin |
| | | factor | | | |
| 3048 | HBG2 | hemoglobin subunit gamma 2 | 8406 | SRPX | sushi repeat containing protein, |
| | | | | | X-linked |
| 27198 | HCAR1 | hydroxycarboxylic acid receptor 1 | 27286 | SRPX2 | sushi repeat containing protein, |
| | | | | | X-linked 2 |
| 338442 | HCAR2 | hydroxycarboxylic acid receptor 2 | 284297 | SSC5D | scavenger receptor cysteine rich |
| | | | | | family member with 5 domains |
| 8843 | HCAR3 | hydroxycarboxylic acid receptor 3 | 54961 | SSH3 | slingshot protein phosphatase 3 |
| 54435 | HCG4 | HLA complex group 4 (non- | 6484 | ST3GAL4 | ST3 beta-galactoside alpha-2,3- |
| | | protein coding) | | | sialyltransferase 4 |
| 3055 | HCK | HCK proto-oncogene, Src family | 8869 | ST3GAL5 | ST3 beta-galactoside alpha-2,3- |
| | | tyrosine kinase | | | sialyltransferase 5 |
| 3059 | HCLS1 | hematopoietic cell-specific Lyn | 6480 | ST6GAL1 | ST6 beta-galactoside alpha-2,6- |
| | | substrate 1 | | | sialyltransferase 1 |
| 10870 | HCST | hematopoietic cell signal | 55808 | ST6GALNAC1 | ST6 N-acetylgalactosaminide |
| | | transducer | | | alpha-2,6-sialyltransferase 1 |
| 9734 | HDAC9 | histone deacetylase 9 | 10610 | ST6GALNAC2 | ST6 N-acetylgalactosaminide |
| | | | | | alpha-2,6-sialyltransferase 2 |
| 3067 | HDC | histidine decarboxylase | 55576 | STAB2 | stabilin 2 |
| 9843 | HEPH | hephaestin | 10734 | STAG3 | stromal antigen 3 |
| 283755 | HERC2P3 | hect domain and RLD 2 | 26228 | STAP1 | signal transducing adaptor family |
| | | pseudogene 3 | | | member 1 |
| 100289574 | HERC2P4 | hect domain and RLD 2 | 55620 | STAP2 | signal transducing adaptor family |
| | | pseudogene 4 | | | member 2 |
| 51191 | HERC5 | HECT and RLD domain | 57519 | STARD9 | StAR related lipid transfer |
| | | containing E3 ubiquitin protein | | | domain containing 9 |
| | | ligase 5 | | | |
| 55008 | HERC6 | HECT and RLD domain | 6772 | STAT1 | signal transducer and activator of |
| | | containing E3 ubiquitin protein | | | transcription 1 |
| | | ligase family member 6 | | | |
| 57801 | HES4 | hes family bHLH transcription | 6775 | STAT4 | signal transducer and activator of |
| | | factor 4 | | | transcription 4 |
| 3082 | HGF | hepatocyte growth factor | 6781 | STC1 | stanniocalcin 1 |
| 64399 | HHIP | hedgehog interacting protein | 26872 | STEAP1 | STEAP family member 1 |
| 283987 | HID1 | HID1 domain containing | 261729 | STEAP2 | STEAP2 metalloreductase |
| 29923 | HILPDA | hypoxia inducible lipid droplet | 55240 | STEAP3 | STEAP3 metalloreductase |
| | | associated | | | |
| 3024 | HIST1H1A | histone cluster 1 H1 family | 79689 | STEAP4 | STEAP4 metalloreductase |
| | | member a | | | |
| 3009 | HIST1H1B | histone cluster 1 H1 family | 202374 | STK32A | serine/threonine kinase 32A |
| | | member b | | | |
| 8335 | HIST1H2AB | histone cluster 1 H2A family | 65975 | STK33 | serine/threonine kinase 33 |
| | | member b | | | |
| 3013 | HIST1H2AD | histone cluster 1 H2A family | 3925 | STMN1 | stathmin 1 |
| | | member d | | | |
| 3012 | HIST1H2AE | histone cluster 1 H2A family | 219736 | STOX1 | storkhead box 1 |
| | | member e | | | |
| 85235 | HIST1H2AH | histone cluster 1 H2A family | 64220 | STRA6 | stimulated by retinoic acid 6 |
| | | member h | | | |
| 8329 | HIST1H2AI | histone cluster 1 H2A family | 412 | STS | steroid sulfatase |
| | | member i | | | |
| 8331 | HIST1H2AJ | histone cluster 1 H2A family | 56670 | SUCNR1 | succinate receptor 1 |
| | | member j | | | |
| 8332 | HIST1H2AL | histone cluster 1 H2A family | 79783 | SUGCT | succinyl-CoA:glutarate-CoA |
| | | member l | | | transferase |
| 8336 | HIST1H2AM | histone cluster 1 H2A family | 23213 | SULF1 | sulfatase 1 |
| | | member m | | | |
| 3018 | HIST1H2BB | histone cluster 1 H2B family | 55959 | SULF2 | sulfatase 2 |
| | | member b | | | |
| 8344 | HIST1H2BE | histone cluster 1 H2B family | 6799 | SULT1A2 | sulfotransferase family 1A |
| | | member e | | | member 2 |
| 8345 | HIST1H2BH | histone cluster 1 H2B family | 6783 | SULT1E1 | sulfotransferase family 1E |
| | | member h | | | member 1 |
| 8346 | HIST1H2BI | histone cluster 1 H2B family | 6820 | SULT2B1 | sulfotransferase family 2B |
| | | member i | | | member 1 |
| 8970 | HIST1H2BJ | histone cluster 1 H2B family | 55061 | SUSD4 | sushi domain containing 4 |
| | | member j | | | |
| 8340 | HIST1H2BL | histone cluster 1 H2B family | 79987 | SVEP1 | sushi, von Willebrand factor type |
| | | member l | | | A, EGF and pentraxin domain |
| | | | | | containing 1 |
| 8342 | HIST1H2BM | histone cluster 1 H2B family | 136306 | SVOPL | SVOP like |
| | | member m | | | |
| 8348 | HIST1H2BO | histone cluster 1 H2B family | 55638 | SYBU | syntabulin |
| | | member o | | | |
| 8350 | HIST1H3A | histone cluster 1 H3 family | 10388 | SYCP2 | synaptonemal complex protein 2 |
| | | member a | | | |
| 8358 | HIST1H3B | histone cluster 1 H3 family | 81493 | SYNC | syncoilin, intermediate filament |
| | | member b | | | protein |
| 8352 | HIST1H3C | histone cluster 1 H3 family | 23345 | SYNE1 | spectrin repeat containing |
| | | member c | | | nuclear envelope protein 1 |
| 8353 | HIST1H3E | histone cluster 1 H3 family | 163183 | SYNE4 | spectrin repeat containing |
| | | member e | | | nuclear envelope family member |
| | | | | | 4 |
| 8968 | HIST1H3F | histone cluster 1 H3 family | 23336 | SYNM | synemin |
| | | member f | | | |
| 8355 | HIST1H3G | histone cluster 1 H3 family | 171024 | SYNPO2 | synaptopodin 2 |
| | | member g | | | |
| 8357 | HIST1H3H | histone cluster 1 H3 family | 6857 | SYT1 | synaptotagmin 1 |
| | | member h | | | |
| 8354 | HIST1H3I | histone cluster 1 H3 family | 255928 | SYT14 | synaptotagmin 14 |
| | | member i | | | |
| 8356 | HIST1H3J | histone cluster 1 H3 family | 83851 | SYT16 | synaptotagmin 16 |
| | | member j | | | |
| 8360 | HIST1H4D | histone cluster 1 H4 family | 51760 | SYT17 | synaptotagmin 17 |
| | | member d | | | |
| 8361 | HIST1H4F | histone cluster 1 H4 family | 9066 | SYT7 | synaptotagmin 7 |
| | | member f | | | |
| 8294 | HIST1H4I | histone cluster 1 H4 family | 90019 | SYT8 | synaptotagmin 8 |
| | | member i | | | |
| 8362 | HIST1H4K | histone cluster 1 H4 family | 84958 | SYTL1 | synaptotagmin like 1 |
| | | member k | | | |
| 8368 | HIST1H4L | histone cluster 1 H4 family | 54843 | SYTL2 | synaptotagmin like 2 |
| | | member l | | | |
| 653604 | HIST2H3D | histone cluster 2 H3 family | 94122 | SYTL5 | synaptotagmin like 5 |
| | | member d | | | |
| 92815 | HIST3H2A | histone cluster 3 H2A | 6866 | TAC3 | tachykinin 3 |
| 128312 | HIST3H2BB | histone cluster 3 H2B family | 10579 | TACC2 | transforming acidic coiled-coil |
| | | member b | | | containing protein 2 |
| 55355 | HJURP | Holliday junction recognition | 4070 | TACSTD2 | tumor associated calcium signal |
| | | protein | | | transducer 2 |
| 3099 | HK2 | hexokinase 2 | 117289 | TAGAP | T-cell activation RhoGTPase |
| | | | | | activating protein |
| 3101 | HK3 | hexokinase 3 | 6876 | TAGLN | transgelin |
| 3105 | HLA-A | major histocompatibility complex, | 6890 | TAP1 | transporter 1, ATP binding |
| | | class I, A | | | cassette subfamily B member |
| 3107 | HLA-C | major histocompatibility complex, | 445347 | TARP | TCR gamma alternate reading |
| | | class I, C | | | frame protein |
| 3108 | HLA-DMA | major histocompatibility complex, | 259289 | TAS2R43 | taste 2 receptor member 43 |
| | | class II, DM alpha | | | |
| 3109 | HLA-DMB | major histocompatibility complex, | 374403 | TBC1D10C | TBC1 domain family member |
| | | class II, DM beta | | | 10C |
| 3112 | HLA-DOB | major histocompatibility complex, | 101060376 | TBC1D3L | TBC1 domain family member 3L |
| | | class II, DO beta | | | |
| 3119 | HLA-DQB1 | major histocompatibility complex, | 90665 | TBL1Y | transducin beta like 1 Y-linked |
| | | class II, DQ beta 1 | | | |
| 3120 | HLA-DQB2 | major histocompatibility complex, | 6899 | TBX1 | T-box 1 |
| | | class II, DQ beta 2 | | | |
| 3123 | HLA-DRB1 | major histocompatibility complex, | 6909 | TBX2 | T-box 2 |
| | | class II, DR beta 1 | | | |
| 3125 | HLA-DRB3 | major histocompatibility complex, | 57057 | TBX20 | T-box 20 |
| | | class II, DR beta 3 | | | |
| 3126 | HLA-DRB4 | major histocompatibility complex, | 6926 | TBX3 | T-box 3 |
| | | class II, DR beta 4 | | | |
| 3128 | HLA-DRB6 | major histocompatibility complex, | 9496 | TBX4 | T-box 4 |
| | | class II, DR beta 6 (pseudogene) | | | |
| 3134 | HLA-F | major histocompatibility complex, | 6910 | TBX5 | T-box 5 |
| | | class I, F | | | |
| 3135 | HLA-G | major histocompatibility complex, | 6920 | TCEA3 | transcription elongation factor A3 |
| | | class I, G | | | |
| 3136 | HLA-H | major histocompatibility complex, | 140597 | TCEAL2 | transcription elongation factor A |
| | | class I, H (pseudogene) | | | like 2 |
| 83872 | HMCN1 | hemicentin 1 | 56849 | TCEAL7 | transcription elongation factor A |
| | | | | | like 7 |
| 3159 | HMGA1 | high mobility group AT-hook 1 | 6943 | TCF21 | transcription factor 21 |
| 3149 | HMGB3 | high mobility group box 3 | 83439 | TCF7L1 | transcription factor 7 like 1 |
| 3158 | HMGCS2 | 3-hydroxy-3-methylglutaryl-CoA | 56892 | TCIM | transcriptional and immune |
| | | synthase 2 | | | response regulator |
| 3161 | HMMR | hyaluronan mediated motility | 6947 | TCN1 | transcobalamin 1 |
| | | receptor | | | |
| 3162 | HMOX1 | heme oxygenase 1 | 6999 | TDO2 | tryptophan 2,3-dioxygenase |
| 6928 | HNF1B | HNF1 homeobox B | 163589 | TDRD5 | tudor domain containing 5 |
| 84525 | HOPX | HOP homeobox | 8463 | TEAD2 | TEA domain transcription factor 2 |
| 84072 | HORMAD1 | HORMA domain containing 1 | 10178 | TENM1 | teneurin transmembrane protein |
| | | | | | 1 |
| 100874323 | HOXA10-AS | HOXA10 antisense RNA | 55714 | TENM3 | teneurin transmembrane protein |
| | | | | | 3 |
| 3207 | HOXA11 | homeobox A11 | 7012 | TERC | telomerase RNA component |
| 3209 | HOXA13 | homeobox A13 | 54997 | TESC | tescalcin |
| 3203 | HOXA6 | homeobox A6 | 7018 | TF | transferrin |
| 3212 | HOXB2 | homeobox B2 | 7020 | TFAP2A | transcription factor AP-2 alpha |
| 3213 | HOXB3 | homeobox B3 | 7022 | TFAP2C | transcription factor AP-2 gamma |
| 3214 | HOXB4 | homeobox B4 | 29842 | TFCP2L1 | transcription factor CP2 like 1 |
| 3215 | HOXB5 | homeobox B5 | 7031 | TFF1 | trefoil factor 1 |
| 3216 | HOXB6 | homeobox B6 | 7033 | TFF3 | trefoil factor 3 |
| 3226 | HOXC10 | homeobox C10 | 7035 | TFPI | tissue factor pathway inhibitor |
| 3221 | HOXC4 | homeobox C4 | 7980 | TFPI2 | tissue factor pathway inhibitor 2 |
| 3223 | HOXC6 | homeobox C6 | 7038 | TG | thyroglobulin |
| 3225 | HOXC9 | homeobox C9 | 7041 | TGFB1I1 | transforming growth factor beta 1 |
| | | | | | induced transcript 1 |
| 3236 | HOXD10 | homeobox D10 | 7043 | TGFB3 | transforming growth factor beta 3 |
| 3237 | HOXD11 | homeobox D11 | 7045 | TGFBI | transforming growth factor beta |
| | | | | | induced |
| 3233 | HOXD4 | homeobox D4 | 7049 | TGFBR3 | transforming growth factor beta |
| | | | | | receptor 3 |
| 3240 | HP | haptoglobin | 7052 | TGM2 | transglutaminase 2 |
| 3242 | HPD | 4-hydroxyphenylpyruvate | 7056 | THBD | thrombomodulin |
| | | dioxygenase | | | |
| 3248 | HPGD | 15-hydroxyprostaglandin | 7057 | THBS1 | thrombospondin 1 |
| | | dehydrogenase | | | |
| 27306 | HPGDS | hematopoietic prostaglandin D | 7058 | THBS2 | thrombospondin 2 |
| | | synthase | | | |
| 60495 | HPSE2 | heparanase 2 (inactive) | 7060 | THBS4 | thrombospondin 4 |
| 55806 | HR | HR, lysine demethylase and | 387357 | THEMIS | thymocyte selection associated |
| | | nuclear receptor corepressor | | | |
| 54979 | HRASLS2 | HRAS like suppressor 2 | 9473 | THEMIS2 | thymocyte selection associated |
| | | | | | family member 2 |
| 90161 | HS6ST2 | heparan sulfate 6-O- | 55258 | THNSL2 | threonine synthase like 2 |
| | | sulfotransferase 2 | | | |
| 3290 | HSD11B1 | hydroxysteroid 11-beta | 7068 | THRB | thyroid hormone receptor beta |
| | | dehydrogenase 1 | | | |
| 3291 | HSD11B2 | hydroxysteroid 11-beta | 79875 | THSD4 | thrombospondin type 1 domain |
| | | dehydrogenase 2 | | | containing 4 |
| 51171 | HSD17B14 | hydroxysteroid 17-beta | 221981 | THSD7A | thrombospondin type 1 domain |
| | | dehydrogenase 14 | | | containing 7A |
| 3294 | HSD17B2 | hydroxysteroid 17-beta | 80731 | THSD7B | thrombospondin type 1 domain |
| | | dehydrogenase 2 | | | containing 7B |
| 8630 | HSD17B6 | hydroxysteroid 17-beta | 7070 | THY1 | Thy-1 cell surface antigen |
| | | dehydrogenase 6 | | | |
| 84941 | HSH2D | hematopoietic SH2 domain | 90381 | TICRR | TOPBP1 interacting checkpoint |
| | | containing | | | and replication regulator |
| 3303 | HSPA1A | heat shock protein family A | 91937 | TIMD4 | T-cell immunoglobulin and mucin |
| | | (Hsp70) member 1A | | | domain containing 4 |
| 3304 | HSPA1B | heat shock protein family A | 7076 | TIMP1 | TIMP metallopeptidase inhibitor 1 |
| | | (Hsp70) member 1B | | | |
| 3306 | HSPA2 | heat shock protein family A | 7077 | TIMP2 | TIMP metallopeptidase inhibitor 2 |
| | | (Hsp70) member 2 | | | |
| 22824 | HSPA4L | heat shock protein family A | 7078 | TIMP3 | TIMP metallopeptidase inhibitor 3 |
| | | (Hsp70) member 4 like | | | |
| 3310 | HSPA6 | heat shock protein family A | 7079 | TIMP4 | TIMP metallopeptidase inhibitor 4 |
| | | (Hsp70) member 6 | | | |
| 3311 | HSPA7 | heat shock protein family A | 64129 | TINAGL1 | tubulointerstitial nephritis antigen |
| | | (Hsp70) member 7 | | | like 1 |
| 3316 | HSPB2 | heat shock protein family B | 27134 | TJP3 | tight junction protein 3 |
| | | (small) member 2 | | | |
| 8988 | HSPB3 | heat shock protein family B | 116238 | TLCD1 | TLC domain containing 1 |
| | | (small) member 3 | | | |
| 126393 | HSPB6 | heat shock protein family B | 7089 | TLE2 | transducin like enhancer of split 2 |
| | | (small) member 6 | | | |
| 27129 | HSPB7 | heat shock protein family B | 79816 | TLE6 | transducin like enhancer of split 6 |
| | | (small) member 7 | | | |
| 26353 | HSPB8 | heat shock protein family B | 7092 | TLL1 | tolloid like 1 |
| | | (small) member 8 | | | |
| 3357 | HTR2B | 5-hydroxytryptamine receptor 2B | 7097 | TLR2 | toll like receptor 2 |
| 3358 | HTR2C | 5-hydroxytryptamine receptor 2C | 51284 | TLR7 | toll like receptor 7 |
| 5654 | HTRA1 | HtrA serine peptidase 1 | 51311 | TLR8 | toll like receptor 8 |
| 94031 | HTRA3 | HtrA serine peptidase 3 | 4071 | TM4SF1 | transmembrane 4 L six family |
| | | | | | member 1 |
| 3382 | ICA1 | islet cell autoantigen 1 | 7108 | TM7SF2 | transmembrane 7 superfamily |
| | | | | | member 2 |
| 3383 | ICAM1 | intercellular adhesion molecule 1 | 147798 | TMC4 | transmembrane channel like 4 |
| 29851 | ICOS | inducible T-cell costimulator | 79838 | TMC5 | transmembrane channel like 5 |
| 3397 | ID1 | inhibitor of DNA binding 1, HLH | 79905 | TMC7 | transmembrane channel like 7 |
| | | protein | | | |
| 3399 | ID3 | inhibitor of DNA binding 3, HLH | 147138 | TMC8 | transmembrane channel like 8 |
| | | protein | | | |
| 3400 | ID4 | inhibitor of DNA binding 4, HLH | 54972 | TMEM132A | transmembrane protein 132A |
| | | protein | | | |
| 3417 | IDH1 | isocitrate dehydrogenase | 135932 | TMEM139 | transmembrane protein 139 |
| | | (NADP(+)) 1, cytosolic | | | |
| 3620 | IDO1 | indoleamine 2,3-dioxygenase 1 | 80008 | TMEM156 | transmembrane protein 156 |
| 8870 | IER3 | immediate early response 3 | 81615 | TMEM163 | transmembrane protein 163 |
| 3429 | IFI27 | interferon alpha inducible protein | 202915 | TMEM184A | transmembrane protein 184A |
| | | 27 | | | |
| 10437 | IFI30 | IFI30, lysosomal thiol reductase | 388564 | TMEM238 | transmembrane protein 238 |
| 10561 | IFI44 | interferon induced protein 44 | 84302 | TMEM246 | transmembrane protein 246 |
| 10964 | IFI44L | interferon induced protein 44 like | 55287 | TMEM40 | transmembrane protein 40 |
| 2537 | IF16 | interferon alpha inducible protein | 55076 | TMEM45A | transmembrane protein 45A |
| | | 6 | | | |
| 64135 | IFIH1 | interferon induced with helicase | 120224 | TMEM45B | transmembrane protein 45B |
| | | C domain 1 | | | |
| 3434 | IFIT1 | interferon induced protein with | 83604 | TMEM47 | transmembrane protein 47 |
| | | tetratricopeptide repeats 1 | | | |
| 3433 | IFIT2 | interferon induced protein with | 84283 | TMEM79 | transmembrane protein 79 |
| | | tetratricopeptide repeats 2 | | | |
| 3437 | IFIT3 | interferon induced protein with | 27346 | TMEM97 | transmembrane protein 97 |
| | | tetratricopeptide repeats 3 | | | |
| 8519 | IFITM1 | interferon induced | 7111 | TMOD1 | tropomodulin 1 |
| | | transmembrane protein 1 | | | |
| 402778 | IFITM10 | interferon induced | 28983 | TMPRSS11E | transmembrane protease, serine |
| | | transmembrane protein 10 | | | 11E |
| 10410 | IFITM3 | interferon induced | 84000 | TMPRSS13 | transmembrane protease, serine |
| | | transmembrane protein 3 | | | 13 |
| 338376 | IFNE | interferon epsilon | 7113 | TMPRSS2 | transmembrane protease, serine |
| | | | | | 2 |
| 3479 | IGF1 | insulin like growth factor 1 | 64699 | TMPRSS3 | transmembrane protease, serine |
| | | | | | 3 |
| 10644 | IGF2BP2 | insulin like growth factor 2 mRNA | 56649 | TMPRSS4 | transmembrane protease, serine |
| | | binding protein 2 | | | 4 |
| 10643 | IGF2BP3 | insulin like growth factor 2 mRNA | 11013 | TMSB15A | thymosin beta 15a |
| | | binding protein 3 | | | |
| 3485 | IGFBP2 | insulin like growth factor binding | 9087 | TMSB4Y | thymosin beta 4, Y-linked |
| | | protein 2 | | | |
| 3486 | IGFBP3 | insulin like growth factor binding | 83857 | TMTC1 | transmembrane and |
| | | protein 3 | | | tetratricopeptide repeat |
| | | | | | containing 1 |
| 3488 | IGFBP5 | insulin like growth factor binding | 3371 | TNC | tenascin C |
| | | protein 5 | | | |
| 3489 | IGFBP6 | insulin like growth factor binding | 7127 | TNFAIP2 | TNF alpha induced protein 2 |
| | | protein 6 | | | |
| 3490 | IGFBP7 | insulin like growth factor binding | 7128 | TNFAIP3 | TNF alpha induced protein 3 |
| | | protein 7 | | | |
| 374918 | IGFL1 | IGF like family member 1 | 7130 | TNFAIP6 | TNF alpha induced protein 6 |
| 147920 | IGFL2 | IGF like family member 2 | 8794 | TNFRSF10C | TNF receptor superfamily |
| | | | | | member 10c |
| 91156 | IGFN1 | immunoglobulin-like and | 4982 | TNFRSF11B | TNF receptor superfamily |
| | | fibronectin type III domain | | | member 11b |
| | | containing 1 | | | |
| 91353 | IGLL3P | immunoglobulin lambda like | 51330 | TNFRSF12A | TNF receptor superfamily |
| | | polypeptide 3, pseudogene | | | member 12A |
| 100423062 | IGLL5 | immunoglobulin lambda like | 23495 | TNFRSF13B | TNF receptor superfamily |
| | | polypeptide 5 | | | member 13B |
| 285313 | IGSF10 | immunoglobulin superfamily | 608 | TNFRSF17 | TNF receptor superfamily |
| | | member 10 | | | member 17 |
| 152404 | IGSF11 | immunoglobulin superfamily | 55504 | TNFRSF19 | TNF receptor superfamily |
| | | member 11 | | | member 19 |
| 3321 | IGSF3 | immunoglobulin superfamily | 27242 | TNFRSF21 | TNF receptor superfamily |
| | | member 3 | | | member 21 |
| 10261 | IGSF6 | immunoglobulin superfamily | 8743 | TNFSF10 | TNF superfamily member 10 |
| | | member 6 | | | |
| 57549 | IGSF9 | immunoglobulin superfamily | 8600 | TNFSF11 | TNF superfamily member 11 |
| | | member 9 | | | |
| 22997 | IGSF9B | immunoglobulin superfamily | 10673 | TNFSF13B | TNF superfamily member 13b |
| | | member 9B | | | |
| 10320 | IKZF1 | IKAROS family zinc finger 1 | 7134 | TNNC1 | troponin C1, slow skeletal and |
| | | | | | cardiac type |
| 22807 | IKZF2 | IKAROS family zinc finger 2 | 7136 | TNNI2 | troponin I2, fast skeletal type |
| 3586 | IL10 | interleukin 10 | 7137 | TNNI3 | troponin I3, cardiac type |
| 3587 | IL10RA | interleukin 10 receptor subunit | 7138 | TNNT1 | troponin T1, slow skeletal type |
| | | alpha | | | |
| 3594 | IL12RB1 | interleukin 12 receptor subunit | 7139 | TNNT2 | troponin T2, cardiac type |
| | | beta 1 | | | |
| 3595 | IL12RB2 | interleukin 12 receptor subunit | 7140 | TNNT3 | troponin T3, fast skeletal type |
| | | beta 2 | | | |
| 3598 | IL13RA2 | interleukin 13 receptor subunit | 7145 | TNS1 | tensin 1 |
| | | alpha 2 | | | |
| 3603 | IL16 | interleukin 16 | 23371 | TNS2 | tensin 2 |
| 55540 | IL17RB | interleukin 17 receptor B | 84951 | TNS4 | tensin 4 |
| 132014 | IL17RE | interleukin 17 receptor E | 7148 | TNXB | tenascin XB |
| 3606 | IL18 | interleukin 18 | 10140 | TOB1 | transducer of ERBB2, 1 |
| 3552 | IL1A | interleukin 1 alpha | 7153 | TOP2A | DNA topoisomerase II alpha |
| 3553 | IL1B | interleukin 1 beta | 9760 | TOX | thymocyte selection associated |
| | | | | | high mobility group box |
| 7850 | IL1R2 | interleukin 1 receptor type 2 | 27324 | TOX3 | TOX high mobility group box |
| | | | | | family member 3 |
| 11141 | IL1RAPL1 | interleukin 1 receptor accessory | 8626 | TP63 | tumor protein p63 |
| | | protein like 1 | | | |
| 26280 | IL1RAPL2 | interleukin 1 receptor accessory | 7164 | TPD52L1 | tumor protein D52 like 1 |
| | | protein like 2 | | | |
| 9173 | IL1RL1 | interleukin 1 receptor like 1 | 7168 | TPM1 | tropomyosin 1 |
| 3557 | IL1RN | interleukin 1 receptor antagonist | 7169 | TPM2 | tropomyosin 2 |
| 53832 | IL20RA | interleukin 20 receptor subunit | 348825 | TPRXL | tetrapeptide repeat homeobox |
| | | alpha | | | like |
| 53833 | IL20RB | interleukin 20 receptor subunit | 7177 | TPSAB1 | tryptase alpha/beta 1 |
| | | beta | | | |
| 3559 | IL2RA | interleukin 2 receptor subunit | 64499 | TPSB2 | tryptase beta 2 |
| | | alpha | | | (gene/pseudogene) |
| 3560 | IL2RB | interleukin 2 receptor subunit | 23430 | TPSD1 | tryptase delta 1 |
| | | beta | | | |
| 3561 | IL2RG | interleukin 2 receptor subunit | 22974 | TPX2 | TPX2, microtubule nucleation |
| | | gamma | | | factor |
| 9235 | IL32 | interleukin 32 | 80342 | TRAF3IP3 | TRAF3 interacting protein 3 |
| 90865 | IL33 | interleukin 33 | 22906 | TRAK1 | trafficking kinesin protein 1 |
| 259307 | IL4I1 | interleukin 4 induced 1 | 50852 | TRAT1 | T-cell receptor associated |
| | | | | | transmembrane adaptor 1 |
| 3569 | IL6 | interleukin 6 | 54210 | TREM1 | triggering receptor expressed on |
| | | | | | myeloid cells 1 |
| 3575 | IL7R | interleukin 7 receptor | 54209 | TREM2 | triggering receptor expressed on |
| | | | | | myeloid cells 2 |
| 9118 | INA | internexin neuronal intermediate | 29953 | TRHDE | thyrotropin releasing hormone |
| | | filament protein alpha | | | degrading enzyme |
| 3624 | INHBA | inhibin beta A subunit | 283392 | TRHDE-AS1 | TRHDE antisense RNA 1 |
| 11185 | INMT | indolethylamine N- | 51127 | TRIM17 | tripartite motif containing 17 |
| | | methyltransferase | | | |
| 8821 | INPP4B | inositol polyphosphate-4- | 8805 | TRIM24 | tripartite motif containing 24 |
| | | phosphatase type II B | | | |
| 79781 | IQCA1 | IQ motif containing with AAA | 23650 | TRIM29 | tripartite motif containing 29 |
| | | domain 1 | | | |
| 80726 | IQCN | IQ motif containing N | 11074 | TRIM31 | tripartite motif containing 31 |
| 10788 | IQGAP2 | IQ motif containing GTPase | 117854 | TRIM6 | tripartite motif containing 6 |
| | | activating protein 2 | | | |
| 128239 | IQGAP3 | IQ motif containing GTPase | 9319 | TRIP13 | thyroid hormone receptor |
| | | activating protein 3 | | | interactor 13 |
| 3656 | IRAK2 | interleukin 1 receptor associated | 7216 | TRO | trophinin |
| | | kinase 2 | | | |
| 3662 | IRF4 | interferon regulatory factor 4 | 10024 | TROAP | trophinin associated protein |
| 3664 | IRF6 | interferon regulatory factor 6 | 8989 | TRPA1 | transient receptor potential cation |
| | | | | | channel subfamily A member 1 |
| 3394 | IRF8 | interferon regulatory factor 8 | 7223 | TRPC4 | transient receptor potential cation |
| | | | | | channel subfamily C member 4 |
| 79191 | IRX3 | iroquois homeobox 3 | 7227 | TRPS1 | transcriptional repressor GATA |
| | | | | | binding 1 |
| 9636 | ISG15 | ISG15 ubiquitin-like modifier | 59341 | TRPV4 | transient receptor potential cation |
| | | | | | channel subfamily V member 4 |
| 3669 | ISG20 | interferon stimulated | 55503 | TRPV6 | transient receptor potential cation |
| | | exonuclease gene 20 | | | channel subfamily V member 6 |
| 3671 | ISLR | immunoglobulin superfamily | 1831 | TSC22D3 | TSC22 domain family member 3 |
| | | containing leucine rich repeat | | | |
| 140862 | ISM1 | isthmin 1 | 57616 | TSHZ3 | teashirt zinc finger homeobox 3 |
| 51477 | ISYNA1 | inositol-3-phosphate synthase 1 | 10103 | TSPAN1 | tetraspanin 1 |
| 22801 | ITGA11 | integrin subunit alpha 11 | 23554 | TSPAN12 | tetraspanin 12 |
| 3673 | ITGA2 | integrin subunit alpha 2 | 10100 | TSPAN2 | tetraspanin 2 |
| 3675 | ITGA3 | integrin subunit alpha 3 | 7105 | TSPAN6 | tetraspanin 6 |
| 3678 | ITGA5 | integrin subunit alpha 5 | 7102 | TSPAN7 | tetraspanin 7 |
| 3655 | ITGA6 | integrin subunit alpha 6 | 7103 | TSPAN8 | tetraspanin 8 |
| 3679 | ITGA7 | integrin subunit alpha 7 | 85453 | TSPYL5 | TSPY like 5 |
| 8516 | ITGA8 | integrin subunit alpha 8 | 650368 | TSSC2 | tumor suppressing |
| | | | | | subtransferable candidate 2 |
| | | | | | pseudogene |
| 3680 | ITGA9 | integrin subunit alpha 9 | 100131187 | TSTD1 | thiosulfate sulfurtransferase like |
| | | | | | domain containing 1 |
| 3683 | ITGAL | integrin subunit alpha L | 22996 | TTC39A | tetratricopeptide repeat domain |
| | | | | | 39A |
| 3687 | ITGAX | integrin subunit alpha X | 319089 | TTC6 | tetratricopeptide repeat domain 6 |
| 3689 | ITGB2 | integrin subunit beta 2 | 7272 | TTK | TTK protein kinase |
| 3690 | ITGB3 | integrin subunit beta 3 | 79739 | TTLL7 | tubulin tyrosine ligase like 7 |
| 3691 | ITGB4 | integrin subunit beta 4 | 7273 | TTN | titin |
| 3694 | ITGB6 | integrin subunit beta 6 | 7846 | TUBA1A | tubulin alpha 1a |
| 3696 | ITGB8 | integrin subunit beta 8 | 7277 | TUBA4A | tubulin alpha 4a |
| 9358 | ITGBL1 | integrin subunit beta like 1 | 7280 | TUBB2A | tubulin beta 2A class IIa |
| 80760 | ITIH5 | inter-alpha-trypsin inhibitor heavy | 347733 | TUBB2B | tubulin beta 2B class IIb |
| | | chain family member 5 | | | |
| 3702 | ITK | IL2 inducible T-cell kinase | 10381 | TUBB3 | tubulin beta 3 class III |
| 9452 | ITM2A | integral membrane protein 2A | 84617 | TUBB6 | tubulin beta 6 class V |
| 81618 | ITM2C | integral membrane protein 2C | 643224 | TUBBP5 | tubulin beta pseudogene 5 |
| 3708 | ITPR1 | inositol 1,4,5-trisphosphate | 7286 | TUFT1 | tuftelin 1 |
| | | receptor type 1 | | | |
| 3713 | IVL | involucrin | 7991 | TUSC3 | tumor suppressor candidate 3 |
| 3714 | JAG2 | jagged 2 | 7294 | TXK | TXK tyrosine kinase |
| 3718 | JAK3 | Janus kinase 3 | 246126 | TXLNGY | taxilin gamma pseudogene, Y- |
| | | | | | linked |
| 58494 | JAM2 | junctional adhesion molecule 2 | 1890 | TYMP | thymidine phosphorylase |
| 83700 | JAM3 | junctional adhesion molecule 3 | 7298 | TYMS | thymidylate synthetase |
| 120425 | JAML | junction adhesion molecule like | 7305 | TYROBP | TYRO protein tyrosine kinase |
| | | | | | binding protein |
| 57608 | JCAD | junctional cadherin 5 associated | 53347 | UBASH3A | ubiquitin associated and SH3 |
| | | | | | domain containing A |
| 3512 | JCHAIN | joining chain of multimeric IgA | 10537 | UBD | ubiquitin D |
| | | and IgM | | | |
| 56704 | JPH1 | junctophilin 1 | 11065 | UBE2C | ubiquitin conjugating enzyme E2 |
| | | | | | C |
| 57158 | JPH2 | junctophilin 2 | 9246 | UBE2L6 | ubiquitin conjugating enzyme E2 |
| | | | | | L6 |
| 3725 | JUN | Jun proto-oncogene, AP-1 | 7345 | UCHL1 | ubiquitin C-terminal hydrolase L1 |
| | | transcription factor subunit | | | |
| 3727 | JUND | JunD proto-oncogene, AP-1 | 7351 | UCP2 | uncoupling protein 2 |
| | | transcription factor subunit | | | |
| 3728 | JUP | junction plakoglobin | 54658 | UGT1A1 | UDP glucuronosyltransferase |
| | | | | | family 1 member A1 |
| 8997 | KALRN | kalirin RhoGEF kinase | 54575 | UGT1A10 | UDP glucuronosyltransferase |
| | | | | | family 1 member A10 |
| 23189 | KANK1 | KN motif and ankyrin repeat | 54659 | UGT1A3 | UDP glucuronosyltransferase |
| | | domains 1 | | | family 1 member A3 |
| 25959 | KANK2 | KN motif and ankyrin repeat | 54657 | UGT1A4 | UDP glucuronosyltransferase |
| | | domains 2 | | | family 1 member A4 |
| 3748 | KCNC3 | potassium voltage-gated channel | 54579 | UGT1A5 | UDP glucuronosyltransferase |
| | | subfamily C member 3 | | | family 1 member A5 |
| 3751 | KCND2 | potassium voltage-gated channel | 54578 | UGT1A6 | UDP glucuronosyltransferase |
| | | subfamily D member 2 | | | family 1 member A6 |
| 3752 | KCND3 | potassium voltage-gated channel | 54577 | UGT1A7 | UDP glucuronosyltransferase |
| | | subfamily D member 3 | | | family 1 member A7 |
| 23704 | KCNE4 | potassium voltage-gated channel | 54576 | UGT1A8 | UDP glucuronosyltransferase |
| | | subfamily E regulatory subunit 4 | | | family 1 member A8 |
| 3755 | KCNG1 | potassium voltage-gated channel | 54600 | UGT1A9 | UDP glucuronosyltransferase |
| | | modifier subfamily G member 1 | | | family 1 member A9 |
| 3757 | KCNH2 | potassium voltage-gated channel | 7366 | UGT2B15 | UDP glucuronosyltransferase |
| | | subfamily H member 2 | | | family 2 member B15 |
| 3772 | KCNJ15 | potassium voltage-gated channel | 7364 | UGT2B7 | UDP glucuronosyltransferase |
| | | subfamily J member 15 | | | family 2 member B7 |
| 3764 | KCNJ8 | potassium voltage-gated channel | 7368 | UGT8 | UDP glycosyltransferase 8 |
| | | subfamily J member 8 | | | |
| 8645 | KCNK5 | potassium two pore domain | 80328 | ULBP2 | UL16 binding protein 2 |
| | | channel subfamily K member 5 | | | |
| 3778 | KCNMA1 | potassium calcium-activated | 201294 | UNC13D | unc-13 homolog D |
| | | channel subfamily M alpha 1 | | | |
| 3779 | KCNMB1 | potassium calcium-activated | 11045 | UPK1A | uroplakin 1A |
| | | channel subfamily M regulatory | | | |
| | | beta subunit 1 | | | |
| 3783 | KCNN4 | potassium calcium-activated | 7348 | UPK1B | uroplakin 1B |
| | | channel subfamily N member 4 | | | |
| 3784 | KCNQ1 | potassium voltage-gated channel | 7379 | UPK2 | uroplakin 2 |
| | | subfamily Q member 1 | | | |
| 3790 | KCNS3 | potassium voltage-gated channel | 7380 | UPK3A | uroplakin 3A |
| | | modifier subfamily S member 3 | | | |
| 343450 | KCNT2 | potassium sodium-activated | 11274 | USP18 | ubiquitin specific peptidase 18 |
| | | channel subfamily T member 2 | | | |
| 11015 | KDELR3 | KDEL endoplasmic reticulum | 162632 | USP32P1 | ubiquitin specific peptidase 32 |
| | | protein retention receptor 3 | | | pseudogene 1 |
| 8284 | KDM5D | lysine demethylase 5D | 8287 | USP9Y | ubiquitin specific peptidase 9, Y- |
| | | | | | linked |
| 10656 | KHDRBS3 | KH RNA binding domain | 7404 | UTY | ubiquitously transcribed |
| | | containing, signal transduction | | | tetratricopeptide repeat |
| | | associated 3 | | | containing, Y-linked |
| 25758 | KIAA1549L | KIAA1549 like | 57216 | VANGL2 | VANGL planar cell polarity |
| | | | | | protein 2 |
| 85449 | KIAA1755 | KIAA1755 | 7409 | VAV1 | vav guanine nucleotide exchange |
| | | | | | factor 1 |
| 9928 | KIF14 | kinesin family member 14 | 7412 | VCAM1 | vascular cell adhesion molecule |
| | | | | | 1 |
| 56992 | KIF15 | kinesin family member 15 | 1462 | VCAN | versican |
| 81930 | KIF18A | kinesin family member 18A | 7422 | VEGFA | vascular endothelial growth |
| | | | | | factor A |
| 10112 | KIF20A | kinesin family member 20A | 79674 | VEPH1 | ventricular zone expressed PH |
| | | | | | domain containing 1 |
| 9493 | KIF23 | kinesin family member 23 | 51442 | VGLLT | vestigial like family member 1 |
| 55083 | KIF26B | kinesin family member 26B | 389136 | VGLL3 | vestigial like family member 3 |
| 11004 | KIF2C | kinesin family member 2C | 50853 | VILL | villin like |
| 24137 | KIF4A | kinesin family member 4A | 7431 | VIM | vimentin |
| 3798 | KIF5A | kinesin family member 5A | 7433 | VIPR1 | vasoactive intestinal peptide |
| | | | | | receptor 1 |
| 3800 | KIF5C | kinesin family member 5C | 7436 | VLDLR | very low density lipoprotein |
| | | | | | receptor |
| 3833 | KIFC1 | kinesin family member C1 | 8875 | VNN2 | vanin 2 |
| 3805 | KIR2DL4 | killer cell immunoglobulin like | 29802 | VPREB3 | V-set pre-B cell surrogate light |
| | | receptor, two Ig domains and | | | chain 3 |
| | | long cytoplasmic tail 4 | | | |
| 3815 | KIT | KIT proto-oncogene receptor | 100128881 | VPS9D1-AS1 | VPS9D1 antisense RNA 1 |
| | | tyrosine kinase | | | |
| 4254 | KITLG | KIT ligand | 54621 | VSIG10 | V-set and immunoglobulin |
| | | | | | domain containing 10 |
| 147700 | KLC3 | kinesin light chain 3 | 23584 | VSIG2 | V-set and immunoglobulin |
| | | | | | domain containing 2 |
| 10365 | KLF2 | Kruppel like factor 2 | 11326 | VSIG4 | V-set and immunoglobulin |
| | | | | | domain containing 4 |
| 9314 | KLF4 | Kruppel like factor 4 | 7447 | VSNL1 | visinin like 1 |
| 688 | KLF5 | Kruppel like factor 5 | 79679 | VTCN1 | V-set domain containing T-cell |
| | | | | | activation inhibitor 1 |
| 687 | KLF9 | Kruppel like factor 9 | 56664 | VTRNA1-1 | vault RNA 1-1 |
| 127707 | KLHDC7A | kelch domain containing 7A | 100126299 | VTRNA2-1 | vault RNA 2-1 |
| 113730 | KLHDC7B | kelch domain containing 7B | 221806 | VWDE | von Willebrand factor D and EGF |
| | | | | | domains |
| 90293 | KLHL13 | kelch like family member 13 | 7454 | WAS | Wiskott-Aldrich syndrome |
| 89857 | KLHL6 | kelch like family member 6 | 374666 | WASH3P | WAS protein family homolog 3 |
| | | | | | pseudogene |
| 3820 | KLRB1 | killer cell lectin like receptor B1 | 100287171 | WASHC1 | WASH complex subunit 1 |
| 3821 | KLRC1 | killer cell lectin like receptor C1 | 57705 | WDFY4 | WDFY family member 4 |
| 3822 | KLRC2 | killer cell lectin like receptor C2 | 116966 | WDR17 | WD repeat domain 17 |
| 3823 | KLRC3 | killer cell lectin like receptor C3 | 284403 | WDR62 | WD repeat domain 62 |
| 8302 | KLRC4 | killer cell lectin like receptor C4 | 144406 | WDR66 | WD repeat domain 66 |
| 10219 | KLRG1 | killer cell lectin like receptor G1 | 256764 | WDR72 | WD repeat domain 72 |
| 100506243 | KRBOX1 | KRAB box domain containing 1 | 58189 | WFDC1 | WAP four-disulfide core domain |
| | | | | | 1 |
| 3848 | KRT1 | keratin 1 | 10406 | WFDC2 | WAP four-disulfide core domain |
| | | | | | 2 |
| 3860 | KRT13 | keratin 13 | 8840 | WISP1 | WNT1 inducible signaling |
| | | | | | pathway protein 1 |
| 3861 | KRT14 | keratin 14 | 8839 | WISP2 | WNT1 inducible signaling |
| | | | | | pathway protein 2 |
| 3866 | KRT15 | keratin 15 | 8838 | WISP3 | WNT1 inducible signaling |
| | | | | | pathway protein 3 |
| 3868 | KRT16 | keratin 16 | 65268 | WNK2 | WNK lysine deficient protein |
| | | | | | kinase 2 |
| 3872 | KRT17 | keratin 17 | 65267 | WNK3 | WNK lysine deficient protein |
| | | | | | kinase 3 |
| 3875 | KRT18 | keratin 18 | 65266 | WNK4 | WNK lysine deficient protein |
| | | | | | kinase 4 |
| 3880 | KRT19 | keratin 19 | 80326 | WNT10A | Wnt family member 10A |
| 54474 | KRT20 | keratin 20 | 7472 | WNT2 | Wnt family member 2 |
| 25984 | KRT23 | keratin 23 | 7482 | WNT2B | Wnt family member 2B |
| 3851 | KRT4 | keratin 4 | 54361 | WNT4 | Wnt family member 4 |
| 3852 | KRT5 | keratin 5 | 7474 | WNT5A | Wnt family member 5A |
| 3853 | KRT6A | keratin 6A | 81029 | WNT5B | Wnt family member 5B |
| 3854 | KRT6B | keratin 6B | 7477 | WNT7B | Wnt family member 7B |
| 3855 | KRT7 | keratin 7 | 7490 | WT1 | Wilms tumor 1 |
| 3856 | KRT8 | keratin 8 | 23286 | WWC1 | WW and C2 domain containing 1 |
| 144501 | KRT80 | keratin 80 | 54739 | XAF1 | XIAP associated factor 1 |
| 440050 | KRTAP5-7 | keratin associated protein 5-7 | 6375 | XCL1 | X-C motif chemokine ligand 1 |
| 57830 | KRTAP5-8 | keratin associated protein 5-8 | 2829 | XCR1 | X-C motif chemokine receptor 1 |
| 3846 | KRTAP5-9 | keratin associated protein 5-9 | 7498 | XDH | xanthine dehydrogenase |
| 200634 | KRTCAP3 | keratinocyte associated protein 3 | 7499 | XG | Xg blood group |
| 388533 | KRTDAP | keratinocyte differentiation | 7503 | XIST | X inactive specific transcript |
| | | associated protein | | | (non-protein coding) |
| 8942 | KYNU | kynureninase | 389668 | XKR9 | XK related 9 |
| 3897 | L1CAM | L1 cell adhesion molecule | 7512 | XPNPEP2 | X-prolyl aminopeptidase 2 |
| 91133 | L3MBTL4 | L3MBTL4, histone methyl-lysine | 7535 | ZAP70 | zeta chain of T-cell receptor |
| | | binding protein | | | associated protein kinase 70 |
| 3898 | LAD1 | ladinin 1 | 114821 | ZBED9 | zinc finger BED-type containing 9 |
| 3902 | LAG3 | lymphocyte activating 3 | 81030 | ZBP1 | Z-DNA binding protein 1 |
| 284217 | LAMA1 | laminin subunit alpha 1 | 7704 | ZBTB16 | zinc finger and BTB domain |
| | | | | | containing 16 |
| 3908 | LAMA2 | laminin subunit alpha 2 | 201501 | ZBTB7C | zinc finger and BTB domain |
| | | | | | containing 7C |
| 3909 | LAMA3 | laminin subunit alpha 3 | 728116 | ZBTB8B | zinc finger and BTB domain |
| | | | | | containing 8B |
| 3910 | LAMA4 | laminin subunit alpha 4 | 57683 | ZDBF2 | zinc finger DBF-type containing 2 |
| 3914 | LAMB3 | laminin subunit beta 3 | 6935 | ZEB1 | zinc finger E-box binding |
| | | | | | homeobox 1 |
| 3918 | LAMC2 | laminin subunit gamma 2 | 79776 | ZFHX4 | zinc finger homeobox 4 |
| 10319 | LAMC3 | laminin subunit gamma 3 | 7538 | ZFP36 | ZFP36 ring finger protein |
| 27074 | LAMP3 | lysosomal associated membrane | 23414 | ZFPM2 | zinc finger protein, FOG family |
| | | protein 3 | | | member 2 |
| 24141 | LAMP5 | lysosomal associated membrane | 7544 | ZFY | zinc finger protein, Y-linked |
| | | protein family member 5 | | | |
| 7805 | LAPTM5 | lysosomal protein | 124220 | ZG16B | zymogen granule protein 16B |
| | | transmembrane 5 | | | |
| 120071 | LARGE2 | LARGE xylosyl- and | 7546 | ZIC2 | Zic family member 2 |
| | | glucuronyltransferase 2 | | | |
| 54900 | LAX1 | lymphocyte transmembrane | 284307 | ZIK1 | zinc finger protein interacting with |
| | | adaptor 1 | | | K protein 1 |
| 3929 | LBP | lipopolysaccharide binding | 84460 | ZMAT1 | zinc finger matrin-type 1 |
| | | protein | | | |
| 3932 | LCK | LCK proto-oncogene, Src family | 163071 | ZNF114 | zinc finger protein 114 |
| | | tyrosine kinase | | | |
| 3934 | LCN2 | lipocalin 2 | 51351 | ZNF117 | zinc finger protein 117 |
| 3936 | LCP1 | lymphocyte cytosolic protein 1 | 7694 | ZNF135 | zinc finger protein 135 |
| 3937 | LCP2 | lymphocyte cytosolic protein 2 | 7710 | ZNF154 | zinc finger protein 154 |
| 11155 | LDB3 | LIM domain binding 3 | 7739 | ZNF185 | zinc finger protein 185 with LIM |
| | | | | | domain |
| 3949 | LDLR | low density lipoprotein receptor | 7757 | ZNF208 | zinc finger protein 208 |
| 23641 | LDOC1 | LDOC1, regulator of NFKB | 7772 | ZNF229 | zinc finger protein 229 |
| | | signaling | | | |
| 116842 | LEAP2 | liver enriched antimicrobial | 113835 | ZNF257 | zinc finger protein 257 |
| | | peptide 2 | | | |
| 3953 | LEPR | leptin receptor | 91975 | ZNF300 | zinc finger protein 300 |
| 3956 | LGALS1 | galectin 1 | 55713 | ZNF334 | zinc finger protein 334 |
| 3957 | LGALS2 | galectin 2 | 147686 | ZNF418 | zinc finger protein 418 |
| 3960 | LGALS4 | galectin 4 | 23090 | ZNF423 | zinc finger protein 423 |
| 8549 | LGR5 | leucine rich repeat containing G | 90594 | ZNF439 | zinc finger protein 439 |
| | | protein-coupled receptor 5 | | | |
| 3972 | LHB | luteinizing hormone beta | 126070 | ZNF440 | zinc finger protein 440 |
| | | polypeptide | | | |
| 10186 | LHFPL6 | LHFPL tetraspan subfamily | 285676 | ZNF454 | zinc finger protein 454 |
| | | member 6 | | | |
| 3976 | LIF | LIF, interleukin 6 family cytokine | 84627 | ZNF469 | zinc finger protein 469 |
| 3977 | LIFR | LIF receptor alpha | 158399 | ZNF483 | zinc finger protein 483 |
| 23547 | LILRA4 | leukocyte immunoglobulin like | 90649 | ZNF486 | zinc finger protein 486 |
| | | receptor A4 | | | |
| 353514 | LILRA5 | leukocyte immunoglobulin like | 25925 | ZNF521 | zinc finger protein 521 |
| | | receptor A5 | | | |
| 79168 | LILRA6 | leukocyte immunoglobulin like | 79818 | ZNF552 | zinc finger protein 552 |
| | | receptor A6 | | | |
| 10859 | LILRB1 | leukocyte immunoglobulin like | 80032 | ZNF556 | zinc finger protein 556 |
| | | receptor B1 | | | |
| 10288 | LILRB2 | leukocyte immunoglobulin like | 57507 | ZNF608 | zinc finger protein 608 |
| | | receptor B2 | | | |
| 11006 | LILRB4 | leukocyte immunoglobulin like | 162963 | ZNF610 | zinc finger protein 610 |
| | | receptor B4 | | | |
| 10990 | LILRB5 | leukocyte immunoglobulin like | 199777 | ZNF626 | zinc finger protein 626 |
| | | receptor B5 | | | |
| 22998 | LIMCH1 | LIM and calponin homology | 389114 | ZNF662 | zinc finger protein 662 |
| | | domains 1 | | | |
| 55679 | LIMS2 | LIM zinc finger domain | 63934 | ZNF667 | zinc finger protein 667 |
| | | containing 2 | | | |
| 8825 | LIN7A | lin-7 homolog A, crumbs cell | 100128252 | ZNF667-AS1 | ZNF667 antisense RNA 1 (head |
| | | polarity complex component | | | to head) |
| 388011 | LINC01550 | long intergenic non-protein | 163223 | ZNF676 | zinc finger protein 676 |
| | | coding RNA 1550 | | | |
| 101927905 | LINC02449 | long intergenic non-protein | 148213 | ZNF681 | zinc finger protein 681 |
| | | coding RNA 2449 | | | |
| 3990 | LIPC | lipase C, hepatic type | 57116 | ZNF695 | zinc finger protein 695 |
| 9388 | LIPG | lipase G, endothelial type | 79986 | ZNF702P | zinc finger protein 702, |
| | | | | | pseudogene |
| 200879 | LIPH | lipase H | 7552 | ZNF711 | zinc finger protein 711 |
| 3993 | LLGL2 | LLGL2, scribble cell polarity | 730087 | ZNF726 | zinc finger protein 726 |
| | | complex component | | | |
| 29995 | LMCD1 | LIM and cysteine rich domains 1 | 442319 | ZNF727 | zinc finger protein 727 |
| 55885 | LMO3 | LIM domain only 3 | 388523 | ZNF728 | zinc finger protein 728 |
| 25802 | LMOD1 | leiomodin 1 | 100129543 | ZNF730 | zinc finger protein 730 |
| 84708 | LNX1 | ligand of numb-protein X 1 | 654254 | ZNF732 | zinc finger protein 732 |
| 100132287 | LOC100132287 | uncharacterized LOC100132287 | 100129842 | ZNF737 | zinc finger protein 737 |
| 101927746 | LOC101927746 | uncharacterized LOC101927746 | 79755 | ZNF750 | zinc finger protein 750 |
| 101927999 | LOC101927999 | putative uncharacterized protein | 729648 | ZNF812P | zinc finger protein 812, |
| | | FLJ44672 | | | pseudogene |
| 101928149 | LOC101928149 | nascent polypeptide-associated | 664701 | ZNF826P | zinc finger protein 826, |
| | | complex alpha subunit | | | pseudogene |
| | | pseudogene | | | |
| 101929819 | LOC101929819 | uncharacterized LOC101929819 | 344787 | ZNF860 | zinc finger protein 860 |
| 102723407 | LOC102723407 | putative V-set and | 169834 | ZNF883 | zinc finger protein 883 |
| | | immunoglobulin domain- | | | |
| | | containing-like protein | | | |
| | | IGHV4OR15-8 | | | |
| 102724660 | LOC102724660 | uncharacterized LOC102724660 | 388559 | ZNF888 | zinc finger protein 888 |
| 102724850 | LOC102724850 | uncharacterized LOC102724850 | 7643 | ZNF90 | zinc finger protein 90 |
| 102724880 | LOC102724880 | uncharacterized LOC102724880 | 7644 | ZNF91 | zinc finger protein 91 |
| 283710 | LOC283710 | uncharacterized LOC283710 | 148198 | ZNF98 | zinc finger protein 98 |

Any of the methods disclosed herein may further include determining the expression level (e.g., the mRNA expression level) of one or more genes or gene signatures.

In some examples, the method further comprises determining the mRNA expression level of one or more of the following gene signatures in the tumor sample from the patient: (a) a luminal signature comprising one or more (e.g., one, two, three, four, five, six, seven, or eight), or all, of keratin 20 (KRT20), peroxisome proliferator activated receptor gamma (PPARG), forkhead box A1 (FOXA1), GATA binding protein 3 (GATA3), sorting nexin 31 (SNX31), uroplakin 1A (UPK1A), uroplakin 2 (UPK2), serine peptidase inhibitor Kazal type 1 (SPINK1), and TOX high mobility group box family member 3 (TOX3); (b) a basal signature comprising one or more (e.g., one, two, three, four, five, six, or seven), or all, of cluster of differentiation 44 (CD44), keratin 5 (KRT5), keratin 6A (KRT6A), keratin 6B (KRT6B), keratin 6C (KRT6C), keratin 14 (KRT14), keratin 16 (KRT16), and collagen type XVII alpha 1 chain (COL17A1); (c) an immune checkpoint signature comprising one or more (e.g., one, two, three, four, five, or six), or all, of cluster of differentiation 274 (CD274), programmed cell death 1 ligand 2 (PDCD1LG2), cytotoxic T-lymphocyte associated protein 4 (CTLA4), programmed cell death protein 1 (PDCD1), lymphocyte activating 3 (LAG3), T cell immunoreceptor with immunoglobulin (lg) and immunoreceptor tyrosine-based inhibitory motif (ITIM) domains (TIGIT), and hepatitis A virus cellular receptor 2 (HAVCR2); (d) a T effector signature comprising one or more (e.g., one, two, three, four, five, six, or seven), or all, of interferon gamma (IFNG), C-X-C motif chemokine ligand 9 (CXCL9), cluster of differentiation 8A (CD8A), granzyme A (GZMA), granzyme B (GZMB), C-X-C motif chemokine ligand 10 (CXCL10), perforin 1 (PRF1), and T-Box transcription factor 21 (TBX21); (e) a natural killer (NK) cell signature comprising one or more (e.g., one, two, three, four, five, or six), or all, of natural killer cell granule protein 7 (NKG7), cluster of differentiation 244 (CD244), natural cytotoxicity triggering receptor 1 (NCR1), killer cell lectin like receptor C2 (KLRC2), killer cell lectin like receptor K1 (KLRK1), cluster of differentiation 266 (CD226), and killer cell immunoglobulin like receptor, two Ig domains and long cytoplasmic tail 4 (KIR2DL4); (f) a general B cell signature comprising one or more (e.g., one, two, or three), or all, of cluster of differentiation 79A (CD79A), cluster of differentiation 79B (CD79B), membrane spanning 4-domains A1 (MS4A1), and V-set pre-B cell surrogate light chain 3 (VPREB3); (g) a plasma cell signature comprising one or more (e.g., one, two, three, four, or five), or all, of marginal zone B and B1 cell specific protein (MZB1), derlin 3 (DERL3), junctional sarcoplasmic reticulum protein 1 (JSRP1), tumor necrosis factor (TNF) receptor superfamily member 17 (TNFRSF17), signaling lymphocytic activation molecule (SLAM) family member 7 (SLAMF7), and immunoglobulin lambda like polypeptide 5 (IGLL5); (h) a myeloid signature comprising one or more (e.g., one, two, three, four, five, or six), or all, of colony stimulating factor 1 receptor (CSF1R), colony stimulating factor 2 receptor subunit alpha (CSF2RA), colony stimulating factor 3 receptor (CSF3R), C-X-C motif chemokine receptor 4 (CXCR4), interleukin 6 receptor (IL6R), macrophage receptor with collagenous structure (MARCO), and cluster of differentiation 14 (CD14); (i) a fibroblast transforming growth factor beta response signature (F-TBRS) comprising one or more (e.g., one, two, three, four, five, six, seven, eight, nine, ten, or eleven), or all, of actin alpha 2, smooth muscle (ACTA2), actin gamma 2, smooth muscle (ACTG2), transgelin (TAGLN), tensin 1 (TNS1), calponin 1 (CNN1), tropomyosin 1 (TPM1), connective tissue growth factor (CTGF), PX domain containing 1 (PXDC1), ADAM metallopeptidase domain 12 (ADAM12), follistatin like 3 (FSTL3), transforming growth factor beta induced (TGFBI), and ADAM metallopeptidase domain 19 (ADAM19); (j) a fatty acid biosynthesis (FAB) signature comprising one or more (e.g., one, two, three, four, five, six, or seven), or all, of acetyl-CoA carboxylase alpha (ACACA), acyl-CoA synthetase long chain family member 3 (ACSL3), fatty acid synthase (FASN), insulin induced gene 1 (INSIG1), SREBF chaperone (SCAP), stearoyl-CoA desaturase (SCD), sterol regulatory element binding transcription factor 1 (SREBF1), and sterol regulatory element binding transcription factor 2 (SREBF2); and/or (k) a UDP glucuronosyltransferase signature (UGT) comprising one or more (e.g., one, two, three, four, five, six, seven, or eight), or all, of UDP glucuronosyltransferase family 1 member A10 (UGT1A10), UDP glucuronosyltransferase family 1 member A8 (UGT1A8), UDP glucuronosyltransferase family 1 member A7 (UGT1A7), UDP glucuronosyltransferase family 1 member A6 (UGT1A6), UDP glucuronosyltransferase family 1 member A5 (UGT1A5), UDP glucuronosyltransferase family 1 member A9 (UGT1A9), UDP glucuronosyltransferase family 1 member A4 (UGT1A4), UDP glucuronosyltransferase family 1 member A1 (UGT1A1), and UDP glucuronosyltransferase family 1 member A3 (UGT1A3).

In some examples, the patient's tumor sample is assigned into the luminal subtype, and the patient's tumor sample has an increased expression level, relative to a reference expression level, of the luminal signature, optionally wherein the patient's tumor sample has an increased expression level, relative to a reference expression level, of the FAB signature and/or UGTs signature, and/or decreased expression levels, relative to reference expression levels, of the basal signature, the immune checkpoint signature, the T effector signature, the NK cell signature, the general B cell signature, the plasma cell signature, the myeloid signature, and/or the F-TBRS.

In some examples, the patient's tumor sample is assigned into the stromal subtype, and the patient's tumor sample has increased expression levels, relative to reference expression levels, of the F-TBRS, optionally wherein the patient's tumor sample has decreased expression levels, relative to reference expression levels, of the basal signature, the immune checkpoint signature, the T effector signature, the NK cell signature, the plasma cell signature, and/or the FAB signature.

In some examples, the patient's tumor sample is assigned into the immune subtype, and the patient's tumor sample has increased expression levels, relative to reference expression levels, of the immune checkpoint signature, the T effector signature, the NK cell signature, the general B cell signature, the plasma cell signature, and/or the myeloid signature, optionally wherein the patient's tumor sample has decreased expression levels, relative to reference expression levels, of the luminal signature, the basal signature, the F-TBRS, the FAB signature, and/or the UGTs signature.

In some examples, the patient's tumor sample is assigned into the basal subtype, and the patient's tumor sample has an increased expression level, relative to a reference expression level, of the basal signature, optionally wherein the patient's tumor sample has decreased expression levels, relative to reference expression levels, of the luminal signature, the general B cell signature, the plasma cell signature, the FAB signature, and/or the UGTs signature.

Any suitable reference expression level for a signature may be used. In some examples, the reference expression level is determined from a population of patients having a previously untreated bladder cancer (e.g., a UC, e.g., a locally advanced or metastatic UC, including in the 1L, 2L, and later (2L+) treatment settings). In some examples, the reference expression level of a signature is the median Z-score of the signature in a population of patients having a UC (e.g., a locally advanced or metastatic UC).

In some examples, the patient's tumor sample is assigned into the immune subtype or the basal subtype, and the patient's tumor sample has (i) an increased expression level, relative to a reference expression level, of PD-L1 in tumor-infiltrating immune cells, tumor cells, or both; or (ii) an increased level, relative to a reference level, of cluster of differentiation 8 (CD8)+ T cell infiltration.

In some examples, the patient's tumor sample is assigned into the basal subtype, and the patient's tumor has an increased level, relative to a reference level, of granulocyte infiltration.

In some examples, assignment of the patient's tumor sample into the basal subtype indicates that the patient is likely to have an increased clinical benefit from treatment with an anti-cancer therapy comprising a PD-1 axis binding antagonist (e.g., atezolizumab or avelumab) compared to a treatment that does not comprise a PD-1 axis binding antagonist (e.g., atezolizumab or avelumab). In some examples, assignment of the patient's tumor sample into the basal subtype indicates that the patient is likely to have an increased clinical benefit from treatment with an anti-cancer therapy comprising atezolizumab compared to a treatment that does not comprise atezolizumab. In some examples, assignment of the patient's tumor sample into the basal subtype indicates that the patient is likely to have an increased clinical benefit from treatment with an anti-cancer therapy comprising avelumab compared to a treatment that does not comprise avelumab. In some examples, the treatment that does not comprise atezolizumab comprises a chemotherapeutic agent (e.g., vinflunine, paclitaxel, or docetaxel) or observation. In some examples, increased clinical benefit comprises a relative increase in one or more of the following: overall survival (OS), objective response rate (ORR), progression-free survival (PFS), complete response (CR), partial response (PR), or a combination thereof. In some examples, increased clinical benefit comprises a relative increase in OS.

In some examples, the patient's tumor sample is assigned into the immune subtype or the basal subtype, and the method further comprises selecting an anti-cancer therapy comprising a PD-1 axis binding antagonist (e.g., atezolizumab or avelumab) for the patient. In some examples, the method further comprises selecting an anti-cancer therapy comprising atezolizumab. In other examples, the method further comprises selecting an anti-cancer therapy comprising avelumab.

In some examples, the patient's tumor sample is assigned into the immune subtype or the basal subtype, and the method further comprises treating the patient by administering an anti-cancer therapy comprising a PD-1 axis binding antagonist (e.g., atezolizumab or avelumab) to the patient. In some examples, the method further comprises treating the patient by administering an anti-cancer therapy comprising atezolizumab to the patient. In other examples, the method further comprises treating the patient by administering an anti-cancer therapy comprising avelumab to the patient.

In some examples, the patient's tumor sample is assigned into the immune subtype or basal subtype, and the method further comprises selecting an anti-cancer therapy comprising a PD-1 axis binding antagonist (e.g., atezolizumab or avelumab) in combination with one or more additional immunotherapy agents (e.g., a cluster of differentiation 28 (CD28) agonist, an OX40 agonist, a glucocorticoid-induced TNFR-related (GITR) agonist, a cluster of differentiation 137 (CD137) agonist, a cluster of differentiation 27 (CD27) agonist, an inducible T-cell costimulator (IC0S) agonist, a herpes virus entry mediator (HVEM) agonist, a natural killer group 2 member D (NKG2D) agonist, a MHC class I polypeptide-related sequence A (MICA) agonist, a natural killer cell receptor 2B4 agonist, a PD-1 axis binding antagonist, a CTLA4 antagonist, a TIM3 antagonist, a B and T lymphocyte associated (BTLA) antagonist, a V-domain Ig suppressor of T cell activation (VISTA) antagonist, a LAG3 antagonist, a B7-H4 antagonist, a cluster of differentiation 96 (CD96) antagonist, a TIGIT antagonist, a cluster of differentiation 226 (CD226) antagonist, a chemokine receptor 8 (CCR8) antagonist, a cancer vaccine, an adoptive cell therapy, or a combination thereof) for the patient. In some examples, the TIGIT antagonist is an anti-TIGIT antibody (e.g., tiragolumab). In some examples, the PD-1 axis binding antagonist or the LAG3 antagonist is an anti-PD-1/anti-LAG3 bispecific antibody.

In some examples, the patient's tumor sample is assigned into the immune subtype or basal subtype, and the method further comprises treating the patient by administering to the patient a PD-1 axis binding antagonist (e.g., atezolizumab or avelumab) in combination with one or more additional immunotherapy agents (e.g., a CD28 agonist, an OX40 agonist, a GITR agonist, a CD137 agonist, a CD27 agonist, an IC0S agonist, an HVEM agonist, an NKG2D agonist, a MICA agonist, a 2B4 agonist, a PD-1 axis binding antagonist, a CTLA4 antagonist, a TIM3 antagonist, a BTLA antagonist, a VISTA antagonist, a LAG3 antagonist, a B7-H4 antagonist, a CD96 antagonist, a TIGIT antagonist, a CD226 antagonist, a CCR8 antagonist, a cancer vaccine, an adoptive cell therapy, or a combination thereof). In some examples, the TIGIT antagonist is an anti-TIGIT antibody (e.g., tiragolumab). In some examples, the PD-1 axis binding antagonist or the LAG3 antagonist is an anti-PD-1/anti-LAG3 bispecific antibody.

In some examples, the immunotherapy agent is an immune checkpoint inhibitor. In some examples, the immunotherapy agent is a CD28, OX40, GITR, CD137, CD27, IC0S, HVEM, NKG2D, MICA, or 2B4 agonist or a CTLA-4, PD-1 axis, TIM-3, BTLA, VISTA, LAG-3, B7H4, CD96, TIGIT, or CD226 antagonist. Other particular immunotherapy agents that may be used include anti-CTLA-4 antibodies or antigen-binding fragments thereof, anti-CD27 antibodies or antigen-binding fragments thereof, anti-CD30 antibodies or antigen-binding fragments thereof, anti-CD40 antibodies or antigen-binding fragments thereof, anti-4-1BB antibodies or antigen-binding fragments thereof, anti-GITR antibodies or antigen-binding fragments thereof, anti-OX40 antibodies or antigen-binding fragments thereof, anti-TRAILR1 antibodies or antigen-binding fragments thereof, anti-TRAILR2 antibodies or antigen-binding fragments thereof, anti-TWEAK antibodies or antigen-binding fragments thereof, anti-TWEAKR antibodies or antigen-binding fragments thereof, anti-BRAF antibodies or antigen-binding fragments thereof, anti-MEK antibodies or antigen-binding fragments thereof, anti-CD33 antibodies or antigen-binding fragments thereof, anti-CD20 antibodies or antigen-binding fragments thereof, anti-CD52 antibodies or antigen-binding fragments thereof, anti-A33 antibodies or antigen-binding fragments thereof, anti-GD3 antibodies or antigen-binding fragments thereof, anti-PSMA antibodies or antigen-binding fragments thereof, anti-Ceacan 1 antibodies or antigen-binding fragments thereof, anti-Galedin 9 antibodies or antigen-binding fragments thereof, anti-HVEM antibodies or antigen-binding fragments thereof, anti-VISTA antibodies or antigen-binding fragments thereof, anti-B7 H4 antibodies or antigen-binding fragments thereof, anti-HHLA2 antibodies or antigen-binding fragments thereof, anti-CD155 antibodies or antigen-binding fragments thereof, anti-CD80 antibodies or antigen-binding fragments thereof, anti-BTLA antibodies or antigen-binding fragments thereof, anti-CD160 antibodies or antigen-binding fragments thereof, anti-CD28 antibodies or antigen-binding fragments thereof, anti-CD226 antibodies or antigen-binding fragments thereof, anti-CEACAM1 antibodies or antigen-binding fragments thereof, anti-TIM3 antibodies or antigen-binding fragments thereof, anti-CD96 antibodies or antigen-binding fragments thereof, anti-CD70 antibodies or antigen-binding fragments thereof, anti-CD27 antibodies or antigen-binding fragments thereof, anti-LIGHT antibodies or antigen-binding fragments thereof, anti-CD137 antibodies or antigen-binding fragments thereof, anti-DR4 antibodies or antigen-binding fragments thereof, anti-CR5 antibodies or antigen-binding fragments thereof, anti-FAS antibodies or antigen-binding fragments thereof, anti-CD95 antibodies or antigen-binding fragments thereof, anti-TRAIL antibodies or antigen-binding fragments thereof, anti-DR6 antibodies or antigen-binding fragments thereof, anti-EDAR antibodies or antigen-binding fragments thereof, anti-NGFR antibodies or antigen-binding fragments thereof, anti-OPG antibodies or antigen-binding fragments thereof, anti-RANKL antibodies or antigen-binding fragments thereof, anti-LTBR antibodies or antigen-binding fragments thereof, anti-BCMA antibodies or antigen-binding fragments thereof, anti-TACI antibodies or antigen-binding fragments thereof, anti-BAFFR antibodies or antigen-binding fragments thereof, anti-EDAR2 antibodies or antigen-binding fragments thereof, anti-TROY antibodies or antigen-binding fragments thereof, and anti-RELT antibodies or antigen-binding fragments thereof.

In some examples, the patient's tumor sample is assigned into the luminal subtype, and the method further comprises selecting an anti-cancer therapy comprising a PD-1 axis binding antagonist (e.g., atezolizumab or avelumab) in combination with one or more additional agents selected from a tyrosine kinase inhibitor (TKI), an FGFR3 antagonist, an anti-HER2 antibody drug conjugate (ADC), an anti-TROP2 ADC, or a combination thereof. In some examples, the patient's tumor sample is assigned into the luminal subtype, and the method further comprises selecting an anti-cancer therapy comprising atezolizumab in combination with one or more additional agents selected from a TKI, an FGFR3 antagonist, an anti-HER2 ADC, an anti-TROP2 ADC, or a combination thereof.

In some examples, the patient's tumor sample is assigned into the luminal subtype, and the method further comprises treating the patient by administering to the patient a PD-1 axis binding antagonist (e.g., atezolizumab or avelumab) in combination with one or more additional agents selected from a TKI, an FGFR3 antagonist, an anti-HER2 ADC, an anti-TROP2 ADC, or a combination thereof. In some examples, the patient's tumor sample is assigned into the luminal subtype, and the method further comprises treating the patient by administering to the patient atezolizumab in combination with one or more additional agents selected from a TKI, an FGFR3 antagonist, an anti-HER2 ADC, an anti-TROP2 ADC, or a combination thereof.

In some examples, the patient's tumor sample is assigned into the stromal subtype, and the method further comprises selecting an anti-cancer therapy comprising a PD-1 axis binding antagonist (e.g., atezolizumab or avelumab) in combination with one or more additional agents selected from a TKI, a TGF-β antagonist, a chemotherapeutic agent, or a combination thereof. In some examples, the patient's tumor sample is assigned into the stromal subtype, and the method further comprises selecting an anti-cancer therapy comprising atezolizumab in combination with one or more additional agents selected from a TKI, a TGF-β antagonist, a chemotherapeutic agent, or a combination thereof.

In some examples, the patient's tumor sample is assigned into the stromal subtype, and the method further comprises treating the patient by administering to the patient a PD-1 axis binding antagonist (e.g., atezolizumab or avelumab) in combination with one or more additional agents selected from a TKI, a TGF-β antagonist, a chemotherapeutic agent, or a combination thereof. In some examples, the patient's tumor sample is assigned into the stromal subtype, and the method further comprises treating the patient by administering to the patient atezolizumab in combination with one or more additional agents selected from a TKI, a TGF-β antagonist, a chemotherapeutic agent, or a combination thereof.

In some examples, the tyrosine kinase inhibitor is a dual EGFR/HER2 tyrosine kinase inhibitor such as lapatinib (TYKERB®, GSK572016 or N-[3-chloro-4-[(3 fluorophenyl) methoxy]phenyl]-6[5[[[2methylsulfonyl)ethyl]amino]methyl]-2-furanyl]-4-quinazolinamine)); an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); or VEGF receptor tyrosine kinase inhibitors such as vatalanib (PTK787/ZK222584, Novartis/Schering AG). In some examples, the TKI may be a receptor tyrosine kinase inhibitor (e.g., a multi-targeted receptor tyrosine kinase inhibitor such as sunitinib or axitinib).

In some examples, the FGFR3 antagonist is an FGFR3 antagonist antibody or a small molecule FGFR3 antagonist. Exemplary FGFR3 antagonist antibodies, such as 184.6, 184.6.1, and 184.6.1N54S, are described, for example, in U.S. Pat. No. 8,410,250, which is incorporated herein by reference in its entirety. In some embodiments, the small molecule FGFR3 antagonist is a tyrosine kinase inhibitor.

In some examples, the anti-HER2 ADC is trastuzumab emtansine (T-DM1, ado-trastuzumab emtansine, KADCYLA®, Genentech), trastuzumab deruxtecan (DS-8201a, T-DXd, ENHERTU®, Gilead), trastuzumab duocarmazine (SYD985, Byondis), A166, XMT-1522, MEDI-4276, ARX788, RC48-ADC, BAT8001, or PF-06804103.

In some examples, the anti-TROP2 ADC is sacituzumab govitecan (TRODELVY®, Gilead), datopotamab deruxtecan (Dato-DXd, DS-1062a, Daiichi Sankyo, AstraZeneca), or BAT8003 (Biothera).

Any of the methods disclosed herein may comprise assaying for somatic alterations in the patient's genotype in the tumor sample obtained from the patient. Any suitable somatic alterations may be assayed. In some examples, the somatic alteration is a short variant, a loss, an amplification, a deletion, a duplication, a rearrangement, or a truncation.

In some examples, the method comprises assaying for somatic alterations in FGFR3, CDKN2A, and/or CDK2NB. In some examples, the patient's tumor sample is assigned into the luminal subtype, and the patient's genotype comprises one or more somatic mutations in FGFR3. In some examples, the patient's tumor sample is assigned into the luminal subtype or the basal subtype, and the patient's genotype comprises a copy-number loss in CDKN2A or CDKN2B.

Any suitable sample may be used for patient classification in the methods described herein. In some examples, the sample is a tumor sample. In some examples, the tumor sample is a formalin-fixed and paraffin-embedded (FFPE) sample, an archival sample, a fresh sample, or a frozen sample. In some examples, the tumor sample is a pre-treatment tumor sample.

In some examples, the patient has a locally advanced UC. In some examples, the patient has a metastatic UC (mUC). In some examples, the patient is previously untreated for the UC. In some examples, the patient is ineligible for a platinum-based chemotherapy. In some examples, the platinum-based chemotherapy comprises cisplatin.

In some examples, the patient has received a previous treatment for the UC. In some examples, the previous treatment for UC comprises a platinum-based chemotherapy. In some examples, the patient's UC had progressed with the platinum-based chemotherapy.

In some examples, the patient has had a cystectomy for the UC.

In some examples, the PD-1 axis binding antagonist (e.g., atezolizumab or avelumab) is administered as a monotherapy. In some examples, the atezolizumab is administered as a monotherapy.

In some examples, the PD-1 axis binding antagonist (e.g., atezolizumab or avelumab) is administered as an adjuvant therapy. In some examples, atezolizumab is administered as an adjuvant therapy. In some examples, a blood sample from the patient is circulating tumor DNA (ctDNA)-positive. In some examples, a blood sample from the patient is circulating tumor DNA (ctDNA)-negative.

In some examples, the method further comprises selecting an additional therapeutic agent to the patient.

In some examples, the method further comprises administering an additional therapeutic agent to the patient.

In some examples, the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof. In some examples, the growth inhibitory agent is a CDK4/6 inhibitor (e.g., palbociclib, ribociclib, or abemaciclib). In some examples, the anti-angiogenic agent is a VEGF antagonist (e.g., any VEGF antagonist disclosed herein, e.g., an anti-VEGF antibody (e.g., bevacizumab) or a tyrosine kinase inhibitor (e.g., sunitinib or axitinib)) or a HIF2A inhibitor (e.g., belzutifan (also known as MK-6482) or PT2385). In some examples, the stromal inhibitor is a TGF-β antagonist (e.g., an anti-TGF-β antibody, e.g., any anti-TGF-β antibody disclosed herein). In some examples, the metabolism inhibitor is a PCSK9 inhibitor (e.g., an anti-PCSK9 antibody, e.g., alirocumab or evolocumab), a FAS inhibitor (e.g., cerulenin, C75, isoniazid, or orlistat (tetrahydrolipstatin)), or an AMPK inhibitor (e.g., SBI-0206965, 5′-hydroxy-staurosporine, or compound C (also known as dorsomorphin)). In some embodiments, the complement antagonist is a C1 inhibitor (e.g., CINRYZE® C1 esterase inhibitor), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB siRNA, Alnylam), or an antisense (e.g., lonis-FB-L_{Rx}), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)).

Any of the methods of classifying a bladder cancer in a patient may further include treating the patient, e.g., using any approach described below in Section III.

### III. Therapeutic Methods, Compositions, and Uses for Bladder Cancer

In one example, provided herein is a method of treating a bladder cancer (e.g., UC, e.g., a locally advanced or metastatic UC, including in the 1L, 2L, and later (2L+) treatment settings) in a human patient, the method comprising: classifying the bladder cancer in the patient according to any one of the methods disclosed herein; and administering an anti-cancer therapy to the patient based on the classification (e.g., into a subtype as disclosed herein).

In another example, provided herein is an anti-cancer therapy for use in treating a bladder cancer (e.g., UC, e.g., locally advanced or metastatic UC, including in the 1L, 2L, and later (2L+) treatment settings) in a human patient, wherein the UC in the patient has been classified (e.g., into a subtype as disclosed herein) according to any one of the methods disclosed herein.

In another example, provided herein is the use of an anti-cancer therapy in the preparation of a medicament for treating a bladder cancer (e.g., UC, e.g., locally advanced or metastatic UC, including in the 1L, 2L, and later (2L+) treatment settings) in a human patient, wherein the UC in the patient has been classified (e.g., into a subtype as disclosed herein) according to any one of the methods disclosed herein.

In some examples, the patient is previously untreated for the bladder cancer, e.g., UC. In some examples, the patient has received a previous treatment for the bladder cancer, e.g., UC.

For example, provided herein is a method of treating a bladder cancer (e.g., UC, e.g., locally advanced or metastatic UC, including in the 1L, 2L, and later (2L+) treatment settings) in a human patient, wherein the patient is previously untreated for the UC, the method comprising: classifying the cancer in the patient according to any one of the methods disclosed herein; and administering an anti-cancer therapy to the patient based on the classification (e.g., into a subtype as disclosed herein).

In another example, provided herein is a method of treating a bladder cancer (e.g., UC, e.g., locally advanced or metastatic UC, including in the 1L, 2L, and later (2L+) treatment settings) in a human patient, wherein the patient has received previous treatment for the UC, the method comprising: classifying the cancer in the patient according to any one of the methods disclosed herein; and administering an anti-cancer therapy to the patient based on the classification (e.g., into a subtype as disclosed herein).

In another example, provided herein is an anti-cancer therapy for use in treating a bladder cancer, e.g., UC (e.g., a locally advanced or metastatic UC) in a human patient, wherein the patient is previously untreated for the UC, wherein the UC in the patient has been classified (e.g., into a subtype as disclosed herein) according to any one of the methods disclosed herein.

In another example, provided herein is an anti-cancer therapy for use in treating a bladder cancer, e.g., UC (e.g., a locally advanced or metastatic UC) in a human patient, wherein the patient has received previous treatment for the UC, wherein the UC in the patient has been classified (e.g., into a subtype as disclosed herein) according to any one of the methods disclosed herein.

In another example, provided herein is the use of an anti-cancer therapy in the preparation of a medicament for treating a bladder cancer, e.g., UC (e.g., a locally advanced or metastatic UC) in a human patient, wherein the patient is previously untreated for the UC, wherein the UC in the patient has been classified (e.g., into a subtype as disclosed herein) according to any one of the methods disclosed herein.

In another example, provided herein is the use of an anti-cancer therapy in the preparation of a medicament for treating a bladder cancer, e.g., UC (e.g., a locally advanced or metastatic UC) in a human patient, wherein the patient has received previous treatment for the UC, wherein the UC in the patient has been classified (e.g., into a subtype as disclosed herein) according to any one of the methods disclosed herein.

In one example, provided herein is a method of treating a locally advanced or metastatic UC in a human patient, the method comprising: classifying the previously untreated locally advanced or metastatic UC in the patient according to any one of the methods disclosed herein; and administering an anti-cancer therapy to the patient based on the classification (e.g., into a subtype as disclosed herein).

In another example, provided herein is a method of treating a locally advanced or metastatic UC in a human patient, the method comprising: classifying the locally advanced or metastatic UC in the patient that has received previous treatment for the UC according to any one of the methods disclosed herein; and administering an anti-cancer therapy to the patient based on the classification (e.g., into a subtype as disclosed herein).

In another example, provided herein is an anti-cancer therapy for use in treating a locally advanced or metastatic UC in a human patient, wherein the previously untreated locally advanced or metastatic UC in the patient has been classified (e.g., into a subtype as disclosed herein) according to any one of the methods disclosed herein.

In another example, provided herein is an anti-cancer therapy for use in treating a locally advanced or metastatic UC in a human patient, wherein the locally advanced or metastatic UC in the patient that has received previous treatment for the UC has been classified (e.g., into a subtype as disclosed herein) according to any one of the methods disclosed herein.

In another example, provided herein is the use of an anti-cancer therapy in the preparation of a medicament for treating a locally advanced or metastatic UC in a human patient, wherein the previously untreated locally advanced or metastatic UC in the patient has been classified (e.g., into a subtype as disclosed herein) according to any one of the methods disclosed herein.

In another example, provided herein is the use of an anti-cancer therapy in the preparation of a medicament for treating a locally advanced or metastatic UC in a human patient, wherein the locally advanced or metastatic UC in the patient that has received previous treatment for the UC has been classified (e.g., into a subtype as disclosed herein) according to any one of the methods disclosed herein.

Any suitable anti-cancer therapy may be administered to the patient based on the classification (e.g., into a subtype as disclosed herein). For example, in some embodiments, a PD-1 axis binding antagonist (e.g., an anti-PD-L1 antibody, e.g., atezolizumab or avelumab) is administered to the patient. In some examples, the anti-cancer therapy comprises atezolizumab. In other examples, the anti-cancer therapy comprises avelumab. In some examples, the method further comprises administering an additional therapeutic agent to the patient.

In some examples, the PD-1 axis binding antagonist is administered in combination with an effective amount of one or more additional therapeutic agents. In some examples, the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof. In some examples, the growth inhibitory agent is a CDK4/6 inhibitor (e.g., palbociclib, ribociclib, or abemaciclib). In some examples, the anti-angiogenic agent is a VEGF antagonist (e.g., any VEGF antagonist disclosed herein, e.g., an anti-VEGF antibody (e.g., bevacizumab) or a tyrosine kinase inhibitor (e.g., sunitinib or axitinib)) or a HIF2A inhibitor (e.g., belzutifan (also known as MK-6482) or PT2385). In some examples, the stromal inhibitor is a TGF-β antagonist (e.g., an anti-TGF-βantibody, e.g., any anti-TGF-β antibody disclosed herein). In some examples, the metabolism inhibitor is a PCSK9 inhibitor (e.g., an anti-PCSK9 antibody, e.g., alirocumab or evolocumab), a FAS inhibitor (e.g., cerulenin, C75, isoniazid, or orlistat (tetrahydrolipstatin)), or an AMPK inhibitor (e.g., SBI-0206965, 5′-hydroxy-staurosporine, or compound C (also known as dorsomorphin)). In some embodiments, the complement antagonist is a C1 inhibitor (e.g., CINRYZE® C1 esterase inhibitor), a C3 inhibitor (e.g., a PEGylated pentadecapeptide (e.g., pegcetacoplan) or an anti-C3 antibody (e.g., H17)), a C5 inhibitor (e.g., an anti-C5 antibody (e.g., eculizumab, ABP959, ALXN1210, ALXN5500, SKY59, or LFG 316), an anti-C5 antibody fragment (e.g., MUBODINA®, a neutralizing mini antibody against C5), an siRNA (e.g., ALNCC5), a recombinant protein (e.g., coversin), or a small molecule (e.g., RA101348)), a C5a receptor antagonist (e.g., PMX53, CCX168, or MP-435), an FD inhibitor (e.g., an anti-FD antibody (e.g., lampalizumab) or a small molecule (e.g., ACH-3856, ACH-4100, or ACH-4471)), an FB inhibitor (e.g., an anti-FB antibody, e.g., TA106), a small molecule (e.g., LNP023), an siRNA (e.g., anti-FB siRNA, Alnylam), or an antisense (e.g., lonis-FB-L_{Rx})), a properdin inhibitor (e.g., an anti-properdin antibody (e.g., NM9401)), a C3 convertase (C3bBb) inhibitor (e.g., an FFH-based protein such as TT30 (CR2/CFH) or mini-FH (Amyndas)), or a C3 convertase (C4bC3B and C3bBb) inhibitor (e.g., mirococept (APT070)).

In any of the preceding examples, each dosing cycle may have any suitable length, e.g., about 7 days, about 14 days, about 21 days, about 28 days, about 35 days, about 42 days, or longer. In some instances, each dosing cycle is about 21 days. In some instances, each dosing cycle is about 42 days.

As a general proposition, the therapeutically effective amount of a PD-1 axis binding antagonist (e.g., atezolizumab) administered to a human will be in the range of about 0.01 to about 50 mg/kg of patient body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In one instance, a PD-1 axis binding antagonist is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, the PD-1 axis binding antagonist may be administered at a dose of about 1000 mg to about 1400 mg every three weeks (e.g., about 1100 mg to about 1300 mg every three weeks, e.g., about 1150 mg to about 1250 mg every three weeks). In some instances, the PD-1 axis binding antagonist may be administered at a dose of 840 mg every two weeks. In some instances, the PD-1 axis binding antagonist may be administered at a dose of 1200 mg every three weeks. In some instances, the PD-1 axis binding antagonist may be administered at a dose of 1680 mg every four weeks.

In some instances, a patient is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

In some instances, atezolizumab is administered to the patient intravenously at a dose of about 840 mg every 2 weeks, about 1200 mg every 3 weeks, or about 1680 mg every 4 weeks. In some instances, atezolizumab is administered to the patient intravenously at a dose of about 840 mg every 2 weeks. In some instances, atezolizumab is administered to the patient intravenously at a dose of about 1200 mg every 3 weeks. In some instances, atezolizumab is administered to the patient intravenously at a dose of about 1680 mg every 4 weeks.

In some instances, atezolizumab is administered at a fixed dose of 1200 mg via intravenous infusion on Days 1 and 22 of each 42-day cycle.

In some instances, avelumab is administered at a dose of 10 mg/kg IV every two weeks.

The PD-1 axis binding antagonist and/or any additional therapeutic agent(s), including an immunotherapy agent, a cytotoxic agent, a growth inhibitory agent, a stromal inhibitor, a metabolism inhibitor, a complement antagonist, a radiation therapy agent, an anti-angiogenic agent (e.g., a VEGF antagonist), or a combination thereof, may be administered in any suitable manner known in the art.

For example, the PD-1 axis binding antagonist and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the patient on the same day.

The PD-1 axis binding antagonist and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus.

Also provided herein are methods for treating bladder cancer (e.g., UC, e.g., a locally advanced or metastatic UC) in a patient comprising administering to the patient a treatment regimen comprising an effective amount of a PD-1 axis binding antagonist (e.g., atezolizumab) and/or in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with bevacizumab, paclitaxel, paclitaxel protein-bound (e.g., nab-paclitaxel), carboplatin, cisplatin, pemetrexed, gemcitabine, etoposide, cobimetinib, vemurafenib, or a combination thereof. The PD-1 axis binding antagonist may be an anti-PD-L1 antibody (e.g., atezolizumab) or an anti-PD-1 antibody.

For example, when administering with chemotherapy, atezolizumab may be administered at a dose of 1200 mg every 3 weeks prior to chemotherapy. In another example, following completion of 4-6 cycles of chemotherapy, atezolizumab may be administered at a dose of 840 mg every 2 weeks, 1200 mg every 3 weeks, or 1680 mg every four weeks. In another example, atezolizumab may be administered at a dose of 840 mg, followed by 100 mg/m² of paclitaxel protein-bound (e.g., nab-paclitaxel); for each 28 day cycle, atezolizumab is administered on days 1 and 15, and paclitaxel protein-bound is administered on days 1, 8, and 15. In another example, when administering with carboplatin and etoposide, atezolizumab can be administered at a dose of 1200 mg every 3 weeks prior to chemotherapy. In yet another example, following completion of 4 cycles of carboplatin and etoposide, atezolizumab may be administered at a dose of 840 mg every 2 weeks, 1200 mg every 3 weeks, or 1680 mg every 4 weeks. In another example, following completion of a 28-day cycle of cobimetinib and vemurafenib, atezolizumab may be administered at a dose of 840 mg every 2 weeks with cobimetinib at a dose of 60 mg orally once daily (21 days on, 7 days off) and vemurafenib at a dose of 720 mg orally twice daily.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### IV. Assessment of PD-L1 Expression

The expression of PD-L1 may be assessed in a patient treated according to any of the methods, compositions for use, and uses described herein. The methods, compositions for use, and uses may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the patient has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table 2 and/or Table 3, respectively.

TABLE 2
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by tumor cells, | |
| associated intratumoral stroma, and contiguous | |
| peri-tumoral desmoplastic stroma | |
| Presence of discernible PD-L1 staining of any | IC2 |
| intensity in tumor-infiltrating immune cells | |
| covering ≥5% to <10% of tumor area occupied by | |
| tumor cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor cells, | |
| associated intratumoral stroma, and contiguous | |
| peri-tumoral desmoplastic stroma | |

TABLE 3
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### V. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′) 2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:

(a) a heavy chain variable region (VH) comprising
the amino acid sequence:
(SEQ ID NO: 9)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,
and
(b) the light chain variable region (VL)
comprising the amino acid sequence:
(SEQ ID NO: 10)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,
and
(b) the light chain amino acid sequence:
(SEQ ID NO: 2)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′) 2 fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′) 2 fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### VI. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier. Any of the additional therapeutic agents described herein may also be included in a pharmaceutical composition or formulation.

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary atezolizumab formulation comprises glacial acetic acid, L-histidine, polysorbate 20, and sucrose, with a pH of 5.8. For example, atezolizumab may be provided in a 20-mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14-mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

### VII. Articles of Manufacture or Kits

Also provided herein are articles of manufacture and kits, which may be used for classifying a patient according to any of the methods disclosed herein.

In one example, provided herein is a kit for classifying a bladder cancer (e.g., UC, e.g., a locally advanced or metastatic UC, including in the 1L, 2L, and later (2L+) treatment settings) in a human patient, the kit comprising: (a) reagents for assaying mRNA in a tumor sample from the patient to provide a transcriptional profile of the patient's tumor; and (b) instructions for assigning the patient's tumor sample into one of the following four subtypes based on the transcriptional profile of the patient's tumor: luminal, stromal, immune, or basal, thereby classifying the UC. Any suitable reagents for assaying mRNA may be included in the kit, e.g., nucleic acids, enzymes, buffers, and the like.

In another aspect, provided herein is an article of manufacture or a kit comprising a PD-1 axis binding antagonist (e.g., atezolizumab). In some instances, the article of manufacture or kit further comprises package insert comprising instructions for using the PD-1 axis binding antagonist to treat or delay progression of bladder cancer (e.g., a locally advanced or metastatic UC, including in the 1L, 2L, and later (2L+) treatment settings) in a patient, e.g., for a patient who has been classified according to any of the methods disclosed herein. In some instances, the article of manufacture or kit further comprises package insert comprising instructions for using the PD-1 axis binding antagonist to treat or delay progression of bladder cancer (e.g., a locally advanced or metastatic UC, including in the 1L, 2L, and later (2L+) treatment settings) in a patient. Any of the PD-1 axis binding antagonists and/or any additional therapeutic agents described herein may be included in the article of manufacture or kits.

In some instances, the PD-1 axis binding antagonist and/or any additional therapeutic agent are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or HASTELLOY®). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agents (e.g., an additional chemotherapeutic agent or anti-neoplastic agent). Suitable containers for the one or more agents include, for example, bottles, vials, bags, and syringes.

Any of the articles of manufacture or kits may include instructions to administer a PD-1 axis binding antagonist, or another anti-cancer therapy, to a patient in accordance with any of the methods described herein, e.g., any of the methods set forth in Section III above.

### EXAMPLES

### Example 1: Molecular Subtypes in Urothelial Carcinoma (UC) Determine Outcome to Checkpoint Blockade

This Example describes an in-depth, multi-omic profiling study involving one of the largest cohorts of patients with UC. Because only a subset of patients responded to PD-L1 blockade by atezolizumab in the IMvigor210, IMvigor211, and IMvigor010 clinical trials, this study aimed to identify the underlying biology associated with response to atezolizumab using multi-omic profiling. To this end, clinical, genomic, and immunohistochemistry (IHC) data were compiled from 1875 patients from three clinical trials: IMvigor210 (NCT02951767 (Cohort 1); NCT02108652 (Cohort 2); phase II atezolizumab monotherapy in first-line (1L)/second-line (2L) metastatic UC), IMvigor211 (NCT02302807; phase III atezolizumab vs. chemotherapy in 2L metastatic UC), and IMvigor010 (NCT02450331; phase III adjuvant atezolizumab vs. observation in non-metastatic UC). Baseline tumor IHC (PD-L1 and CD8), bulk RNA-seq, and somatic mutation profiling (either by whole exome sequencing (WES) or the Foundation Medicine FOUNDATIONONE® CDx comprehensive genomic profiling assay) were conducted. In addition, patients from IMvigor010 were profiled by circulating tumor DNA (ctDNA) post-cystectomy and analyzed separately based on ctDNA positivity before adjuvant treatment.

### A. Study Design and Rationale

In the two-cohort, multicenter, phase II IMvigor210 trial, 310 patients who had previously received platinum treatment showed significantly improved objective response rate (ORR) compared to historical controls (15% vs. 10%, p=0.0058) (Rosenberg et al. Lancet. 387:1909-1920 (2016); Mariathasan et al. Nature. 554:544-548 (2018)). Furthermore, the responses were durable in 38 of 45 responders (median follow-up: 11.7 months), and the safety profile was favorable with no treatment-related deaths.

The results from the IMvigor210 trial led to the multicenter, randomized, controlled, phase III IMvigor211 study comparing atezolizumab to chemotherapy in 931 patients with locally advanced or metastatic UC following progression with platinum-based chemotherapy (Powles et al. Lancet. 391:748-757 (2018)). In the PD-L1 positive (PD-L1 IC>5%) population (n=234), although atezolizumab treatment was not associated with significantly longer overall survival (OS) compared to chemotherapy (median 11.1 months vs. 10.6 months; p=0.41), patients in the atezolizumab group had longer durable responses (median 15.9 months vs. 8.3 months) and favorable safety profiles confirming the results of the IMvigor210 study.

Atezolizumab was also tested as an adjuvant therapy in the non-metastatic setting in IMvigor010, a randomized phase III trial comparing atezolizumab to observation following cystectomy (Powles et al. Nature. 595:432-437 (2021)). While atezolizumab benefit was not observed in the intent-to-treat population, disease-free survival (DFS) (HR=0.58, CI: 0.43-0.79, P=0.0024) and OS (HR=0.59, CI: 0.41-0.86) benefit was observed in patients who were ctDNA-positive after cystectomy.

In the above-mentioned trials, not all patients showed improved clinical outcomes in response to PD-L1 blockade. Consequently, the present study was performed to understand the underlying biology associated with response to atezolizumab in patients from the IMvigor210, IMvigor211, and IMvigor010 clinical trials.

### B. Materials and Methods

i. Patients

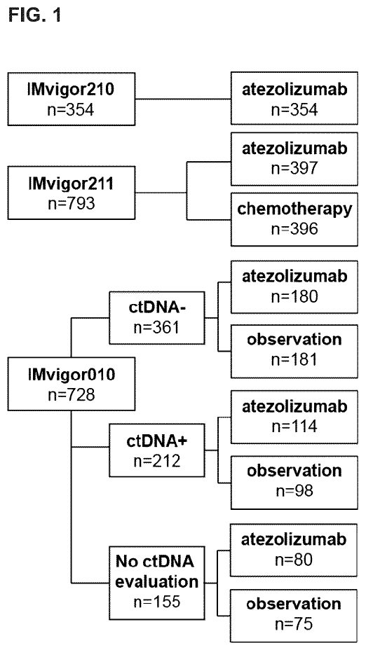

Patients included in this study were participants of the IMvigor210, IMvigor211, and IMvigor010 clinical trials (FIG. 1). In this study, atezolizumab-treated patients (n=357) were included from the IMvigor210 phase II trial (Rosenberg et al. Lancet. 387:1909-1920 (2016); Mariathasan et al. Nature. 554:544-548 (2018)), and atezolizumab-(n=397) and chemotherapy-treated patients (n=396) were included from the IMvigor211 phase III clinical trial (Powles et al. Lancet. 391:748-757 (2018)). This study also included patients from the IMvigor010 phase III clinical trial who were (1) identified as negative for ctDNA (ctDNA−), (2) identified as positive for ctDNA (ctDNA+), and (3) not evaluated for ctDNA status (Powles et al. Nature. 595:432-437 (2021)). The three groups from the IMvigor010 trial included atezolizumab and observation arm patients (FIG. 1).

ii. RNA and DNA Sample Procurement and Processing

Formalin-fixed paraffin-embedded (FFPE) tissue was macro-dissected for tumor area using hematoxylin and eosin (H&E) staining as a guide. RNA was extracted using the High Pure FFPET RNA Isolation Kit (Roche) and assessed by QUBIT™ (Thermo Fisher Scientific) and Agilent Bioanalyzer for quantity and quality. First-strand cDNA synthesis was primed from total RNA using random primers, followed by the generation of second-strand cDNA with dUTP in place of dTTP in the master mix to facilitate preservation of strand information. Libraries were enriched for the mRNA fraction by positive selection using a cocktail of biotinylated oligonucleotides corresponding to coding regions of the genome. Libraries were sequenced using sequencing by synthesis (SBS) technology (ILLUMINA®).

iii. RNA-Seq Data Generation and Processing

Raw RNA-seq counts were obtained from Genentech's internal stranded count pipeline. Raw counts were adjusted for gene length using transcript-per-million (TPM) normalization, and subsequently log 2-transformed to obtain processed data.

iv. Non-Negative Matrix Factorization (NMF)

Using Median Absolute Deviation (MAD) analysis, 3072 genes (top 10%) were selected with the highest variability across patients (Table 1). Subclasses were then computed by reducing the dimensionality of the expression data from thousands of genes to a few metagenes using consensus NMF clustering (Brunet et al. Proc Natl Acad Sci USA. 101:4164-4169 (2004)). This method computes multiple k-factor factorization decompositions of the expression matrix and evaluates the stability of the solutions using a cophenetic coefficient. The most robust consensus NMF clustering of 1875 patient samples using the 3072 most variable genes selected and testing k=2 to k=8 was identified for k=4.

v. Development of Molecular Subtype Classifier Using Random Forest

A machine learning-based classifier was developed based on the random forest machine learning algorithm to derive a robust gene expression-based classifier to predict the NMF clusters in an independent data set. A random forest classifier involves learning a large number of binary decision trees from random subsets of a training set. These trees in the classifier can then be used in a prediction algorithm to identify the similarity of a given sample to a given class in the training set. Before learning the random forest classifier, the data was preprocessed to generate the training set. To ensure accurate prediction of all four NMF classes, the data was down-sampled by randomly removing observation from the majority classes to prevent its signal from dominating the learning algorithm. The gene expression values were also normalized (z-score transformed).

vi. PD-L1 Immunohistochemistry and Classification

PD-L1 expression was assessed by immunohistochemistry (IHC) using the SP142 clone (VENTANA). Tumors were characterized as PD-L1+ if PD-L1 staining of any intensity on immune cells covered ≥1% of tumor area occupied by tumor cells, associated intratumoral, and contiguous peritumoral desmoplastic stroma. All other tumors were characterized as PD-L1−.

vii. DNA Mutation and Copy-Number Profiling by FOUNDATIONONE® Assay

Comprehensive genomic profiling (CGP) was carried out in a Clinical Laboratory Improvement Amendments (CLIA)-certified, College of American Pathologists (CAP)-accredited laboratory (Foundation Medicine Inc., Cambridge, MA) on all-comers during the course of routine clinical care. Approval was obtained from the Western Institutional Review Board (Protocol No. 20152817). Hybrid capture was carried out for all coding exons from up to 324 cancer-related genes plus select introns from up to 31 genes frequently rearranged in cancer. All classes of genomic alterations (GA) were assessed including short variant, copy number, and rearrangement alterations, as described previously (Frampton et al. Nat Biotechnol. 31:1023-1031 (2013)). Biallelic (CN=0) copy number loss was called as previously described (Frampton et al. Nat Biotechnol. 31:1023-1031 (2013)). Shallow copy-number loss (CN=1) was called using similar methodology to arm-level calling. Normalized coverage data for exonic, intronic, and single nucleotide polymorphism (SNP) targets accounting for stromal admixture were plotted on a logarithmic scale and minor allele SNP frequencies were concordantly plotted. Custom circular binary segmentation further clustered targets and minor allele SNPs to define upper and lower bounds of genomic segments. Signal-to-noise ratios for each segment were used to determine whether it was gained or lost. The sum of those segment sizes determined the fraction of each segment gained or lost. For mutation analyses, position-level information was leveraged to define per-gene alteration profiles, and every gene's mutational profile was dichotomized as mutated (including copy-number loss or gain) or not mutated.

viii. Signature Scores

Signature scores were calculated as the median z-score of genes included in each signature for each sample. When summarized by patient group, log 2-transformed expression data was first aggregated by patient group using the median, and subsequently converted to a group z-score.

### C. Results

### i. Identification of Four Molecular Subtypes of Urothelial Carcinoma (UC)

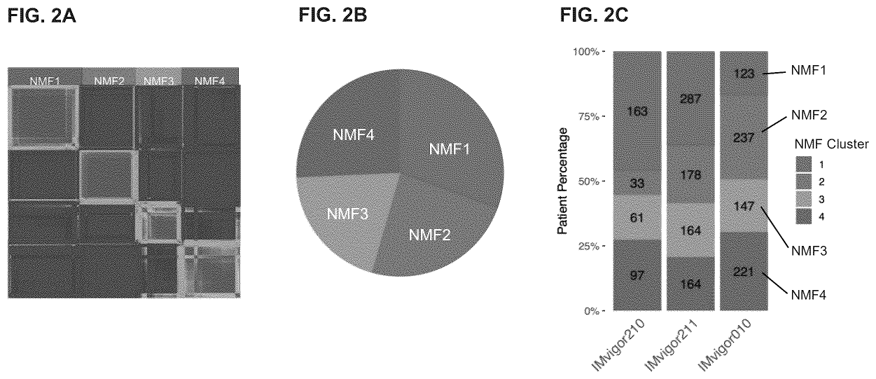

To identify transcriptionally-defined subgroups of patients in an unbiased way, an unsupervised and unbiased machine learning algorithm based on publicly available non-negative matrix factorization (NMF; Brunet et al. Proc Natl Acad Sci USA. 101:4164-4169 (2004)) was applied to the RNA-seq data. This approach yielded four transcriptionally-defined clusters of patient tumors, with distinct biology, enrichment in somatic alterations, and associations with clinical outcomes (FIGS. 2A-2C).

### ii. Molecular Subtype Association with Established Biomarkers of Response in UC

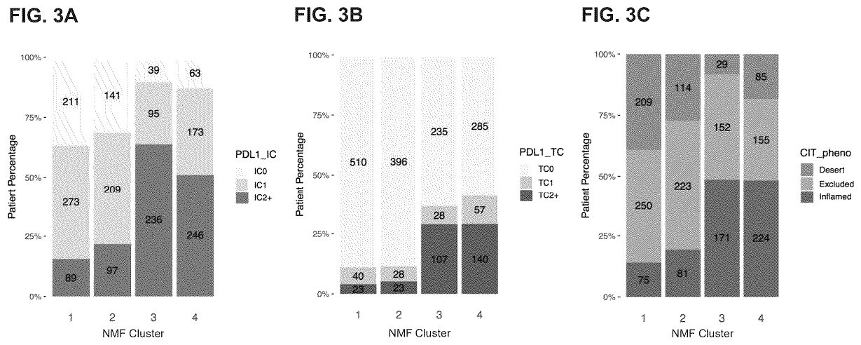

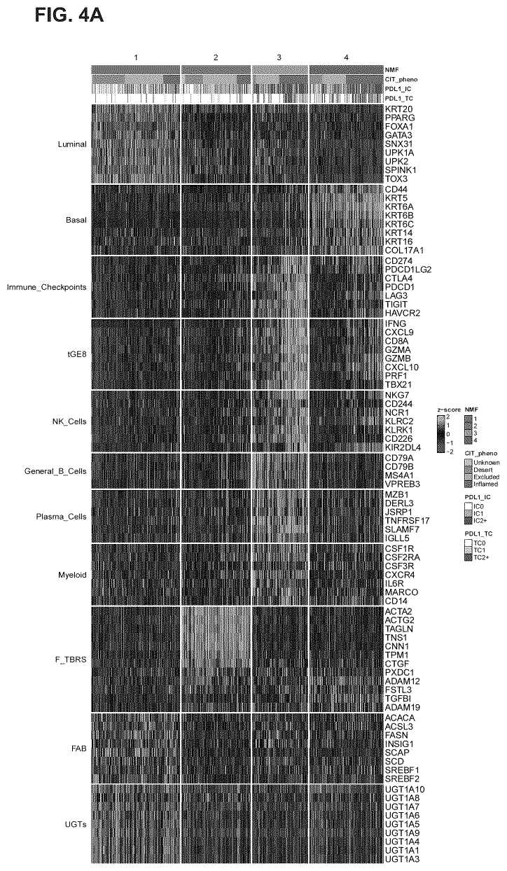

The biological makeup of clusters was determined by a combination of known biomarker enrichment (i.e., PD-L1 expression; tumor immune phenotype defined by CD8), linear modeling on transcription data, and pathway enrichment analysis. Immune phenotype is classified into desert, excluded and inflamed tumors, based on assessment of CD8 IHC staining patterns by a trained pathologist. Desert tumors were largely devoid of CD8+ T cells, excluded tumors exhibited CD8+ T cell accumulation outside the stroma, with low infiltrate into the tumor compartment, while inflamed tumors showed infiltration of CD8+ T cells inside the tumor compartment (Hegde and Chen. Immunity. 52:17-35 (2020)). Both NMF3 and NMF4 exhibited high PD-L1 expression on immune and tumor cells (FIGS. 3A and 3B), and high CD8+ T cell infiltrate as compared to NMF1 and NMF2 (FIG. 3C). These results demonstrate that the molecular subtypes defined by NMF clustering were associated with established biomarkers of response in UC, including PD-L1 expression and immunological tumor subgroups.

### iii. Molecular Subtype Association with Transcriptomic-Based Signatures and Genomic Markers

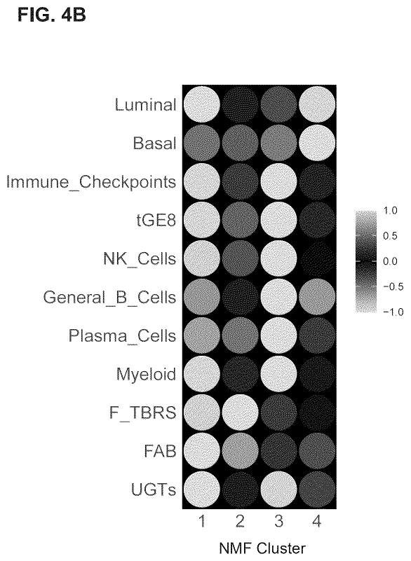

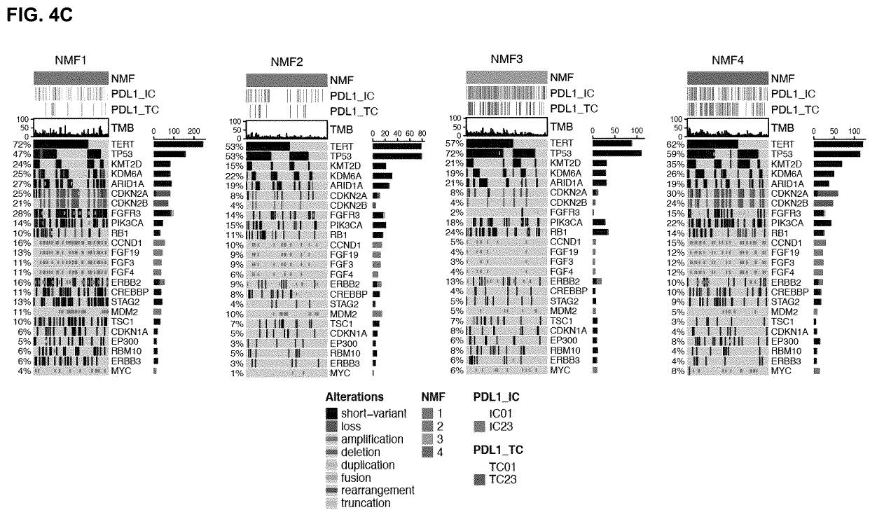

To understand the biological features driving the NMF clusters, transcriptional profiles of the NMF clusters were summarized at both gene- (FIG. 4A) and signature-levels (FIG. 4B). In addition, the transcriptional profiles were complemented by the evaluation of genomic alterations (FIG. 4C). NMF1 was a cluster enriched for luminal signals, fatty acid biosynthesis (FAB), and UDP glucuronosyltransferase (UGTs), with low immune infiltrate and increased frequency of FGFR3 mutations leading to amplified FGFR3 transcription. NMF2 was a cluster enriched for stromal signals, including TGF-β-induced signature, fibroblasts, and endothelial cells. NMF3 was highly enriched for immune signals, including myeloid and lymphoid (T cell and B cell) signatures. Finally, NMF4 was enriched for a basal signature, with intermediate immune infiltrate and low B cell signature. Pathology review of H&E slides also identified increased granulocyte infiltrate in NMF4. Both NMF1 and NMF4 exhibited increased copy-number loss in the CDKN2A/B locus. Based on these findings, NMF1 was annotated as luminal, NMF2 as stromal, NMF3 as immune, and NMF4 as basal.

### iv. UC Molecular Subtypes Associate with Differential Clinical Outcomes to Atezolizumab and Chemotherapy

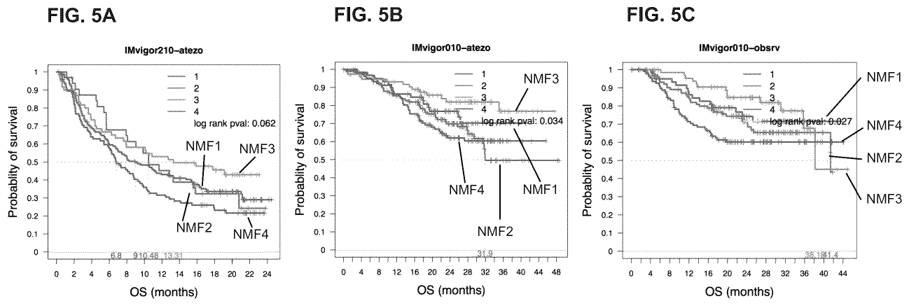

To characterize the clinical outcomes in the IMvigor210 and IMvigor010 clinical trials by NMF clustering, the OS of patients in each NMF cluster was compared. For IMvigor210 and IMvigor010, NMF3 (immune) patients exhibited the longest OS in response to atezolizumab, while those from NMF4 (basal) exhibited the shortest OS, suggesting poor prognosis for patients with basal tumors (FIGS. 5A-5C). In IMvigor010 (FIG. 5B), NMF2 also exhibited shorter OS in the atezolizumab arm relative to the other molecular subtypes.

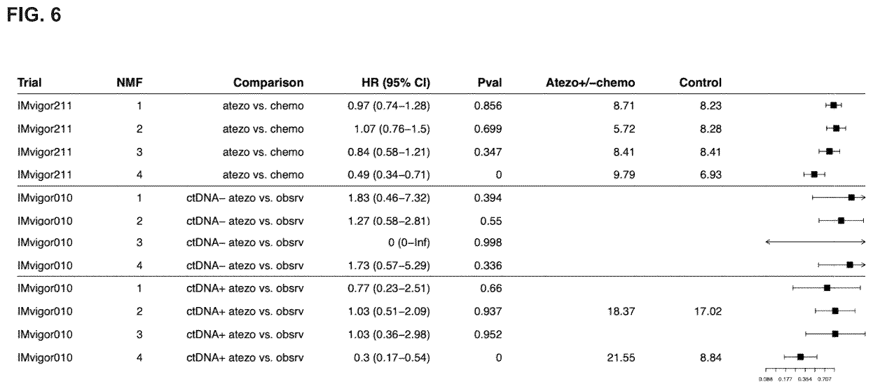

Furthermore, when comparing treatment arms in IMvigor211 and IMvigor010 (ctDNA+ population), NMF4 patients exhibited increased OS when treated with atezolizumab vs. chemotherapy (IMvigor211) or under observation (IMvigor010) (FIG. 6), suggesting a predictive value of this stratification scheme in this patient subset.

Based on the transcriptional clusters obtained, a proprietary random-forest algorithm has been developed that currently uses 576 genes to enable prospective assignment of UC patients to any of the four subsets. An exemplary proposed treatment scheme is shown in Table 4. Chemotherapy may comprise cisplatin for patients who are eligible, or carboplatin for those who are ineligible for cisplatin. Tyrosine kinase inhibitors (TKI) may comprise cabozantinib or axitinib. This stratification algorithm can be utilized to inform treatment selection and new checkpoint inhibitor (CPI) combinations in future prospective studies.

TABLE 4
Molecular Subtyping by RNA-seq

| | | | |
|---|---|---|---|
| NMF1 | NMF2 | NMF3 | NMF4 |
| Luminal | Stromal | Immune | Basal |

| | | |
|---|---|---|
| TKI | TKI | IO/IO combinations |
| FGFR3i | TGFbi | anti-TIGIT + |
| HER2-ADC | Chemo + | Atezo |

| | |
|---|---|
| TROP2-ADC + | Atezo |
| Atezo | |

TKI, tyrosine kinase inhibitor; FGFR3i, FGFR3 inhibitor; ADC, antibody-drug conjugate; atezo, atezolizumab; TGFbi, TGF-β inhibitor; chemo, chemotherapy; IO, immuno-oncology.

### v. Summary

Overall, this study in 1875 patients across three clinical trials, two of them in randomized settings, generated a robust classification scheme that provides prognostic and predictive value in the context of checkpoint inhibition, e.g., for anti-cancer therapies that include atezolizumab. It will inform treatment selection in prospective studies and help identify new combination partners for patient groups that do not benefit from atezolizumab monotherapy in UC.

### Example 2: Molecular Heterogeneity in Urothelial Carcinoma (UC) Determines Clinical Benefit to PD-L1 Blockade

### A. Materials and Methods

i. Development of a Molecular Subtype Classifier Using Random Forest

A machine learning based classifier was developed based on the random forest machine learning algorithm to derive a robust gene expression-based classifier that can predict NMF cluster category in single individuals in independent datasets. A random forest classifier involves learning a large number of binary decision trees from random subsets of a training set. These trees in the classifier can then be used in a prediction algorithm to identify the similarity of a given sample to a given class in the training set. Before learning the random forest classifier, we preprocessed the data to generate the training set. To ensure accurate prediction of all four NMF classes, we down-sampled 1875 patient samples from the NMF discovery cohort to 1488 samples with 372 samples in each NMF class by randomly removing observation from the majority classes to prevent its signal from dominating the learning algorithm. We also normalized (z-score transformed) the gene expression values. We trained the random forest classifier on the 1488 samples and then used the classifier to predict the NMF classes in the validation cohort (IMvigor130; N=928). In the hold out set not included in the training, the accuracy was 91.2% (353/387).

ii. Digital Pathology

A total of 2816 patients, with availability of corresponding digitized hematoxylin and eosin (H&E) stained whole slide images (WSI) at 40× magnification were curated across IMvigor210/211/130/010 for digital pathology image analysis. Human interpretable image features (HIFs) that investigate the spatial heterogeneity and cellular composition of the tumor microenvironment were extracted from these H&E images using three main deep learning models developed in collaboration with PathAI. First, a trained “artifact detection” model was deployed on the H&E stained WSI to predict and exclude tissue regions with distortions such as folding and blurring from further analysis. Next, an already developed and validated “tissue detection” model was used to classify the remaining viable tissue (without imaging artifacts or scanned background) into cancer epithelium, stroma, necrotic regions or normal tissue. Finally, PathAI's “cell-type” model was used to identify the cells in each tissue region and label them as lymphocytes, fibroblasts, macrophages or cancer cells (Diao et al. Nat. Commun. 12:2506 (2021)). Using these tissue region segmentations and cell entities, a total of 424 HIFs were extracted from one representative (with the largest area of cancer epithelium) H&E WSI each from 2816 patients across IMvigor210/211/130/010 (Table 5). 1957 patients and their corresponding H&E WSI were used as the discovery cohort to identify distinct HIFs that were representative for each of the 4 UC subtypes and 859 patients were used in the validation cohort.

TABLE 5
Data distribution for the corresponding
digital pathology analysis in H&E WSI

| Cohort | Datasets | NMF1 | NMF2 | NMF3 | NMF4 | Total |
|---|---|---|---|---|---|---|
| Discovery | IMvigor211 | 285 | 176 | 161 | 160 | 782 |
| (n = 1957) | IMvigor010 | 123 | 237 | 147 | 221 | 728 |
| | IMvigor210 | 160 | 32 | 59 | 96 | 347 |
| Validation | IMvigor130 | 310 | 181 | 176 | 192 | 859 |
| (n = 859) | | | | | | |

For each UC subtype, univariate analysis with one-sided Mann Whitney U test with Bonferroni correction for multiple comparisons was performed in a one-vs-rest setting to identify distinct HIFs that were associated in that UC subtype (p<0.5). For instance, 19 unique features were found to be expressed higher in the NMF1 subtype compared to the rest of the UC subtypes. Similarly, 16, 28 and 13 unique features were found to be expressed higher in NMF2, NMF3 and NMF4 subtypes respectively within the discovery cohort and also verified in the validation cohort. The analysis for this experiment was performed using statistical functions (scipy.stats and statsmodel.stats.multitest packages) in Python 3.9.7.

iii. Single Cell RNAseq Analysis

Single cell analysis of bladder cancer tumors was performed using publicly available data obtained from Gene Expression Omnibus (GEO) with accession number GSE211388 (Yu et al. Mol. Cancer Ther. 21:1729-1741 (2022)) or from the supplementary data from Chen et al. (Chen et al. Nat. Commun. 11:5077 (2020)). Standard preprocessing of raw counts was done using Seurat to normalize and scale each dataset individually. For each dataset, variable features were then identified using the Seurat function VariableFeatures with the “vst” selection method. Next, principal component analysis was performed using these variable features, and the first 20 PCA dimensions were retained for identifying Shared Nearest Neighbors, cell clustering and generating a uniform manifold approximation and projection (UMAP) for visualization (Seurat RunPCA, FindNeighbors, FindClusters). After these preprocessing steps, the annotations provided by the authors were used to filter out non-epithelial cells. Seurat SelectIntegrationFeatures (2000 features), FindIntegrationAnchors and IntegrateData were used to integrate both datasets. Finally, preprocessing of the integrated dataset was done as described above and integrated feature counts were used for plotting.

### B. Results

i. Patient and Biomarker Collections

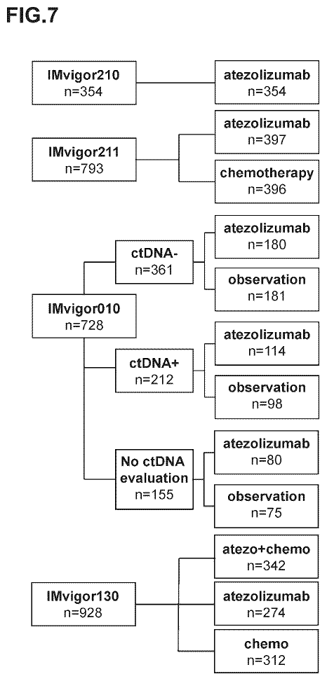

Pre-treatment tumors from 2,803 patients from four clinical trials in locally advanced or metastatic (IMvigor210: n=354; IMvigor211: n=793; IMvigor130: n=928) and muscle invasive non-metastatic (IMvigor010: n=728) UC were analyzed in this study (FIG. 7). IMvigor210 is a single arm Phase 2 trial of atezolizumab in 1L/2L+ locally advanced or metastatic patients (Rosenberg et al. Lancet. 387:1909-1920 (2016), Balar et al. Lancet. 389:67-76 (2017)). IMvigor211 is a randomized Phase 3 trial comparing atezolizumab to chemotherapy in 2L+ locally advanced or metastatic UC patients (Powles et al. Lancet. 391:748-757 (2018)). IMvigor130 is a randomized Phase 3 trial comparing atezolizumab, atezolizumab+chemotherapy and chemotherapy alone in 1L locally advanced or metastatic UC patients (Galsky et al. Lancet. 395:1547-1557 (2020)). IMvigor010 is a randomized Phase 3 trial comparing atezolizumab to observation in adjuvant settings in muscle invasive non-metastatic UC (Powles et al. Nature. 595:432-437 (2021)). Circulating tumor DNA (ctDNA) analysis was conducted in a subset of IMvigor010 patients, to identify patients at risk of relapse following cystectomy. In this combined analysis, we considered overall survival (OS) as the common clinical endpoint between the four trials. All 2,803 pre-treatment tumors were transcriptionally profiled by bulk RNAseq. Of these, 2,168 tumors were also assessed for somatic alterations (IMvigor210: n=276; IMvigor211: n=566; IMvigor130: n=887; IMvigor010: n=439) using a targeted panel of 324 genes (FOUNDATIONONE®). Tumors were also assessed for PD-L1 expression on immune (IC) and tumor (TC) cells, and CD8+ T cell inflamed, excluded or desert phenotypes (Hegde and Chen. Immunity. 52:17-35 (2020)) by immunohistochemistry.

ii. Four Molecular UC Subtypes

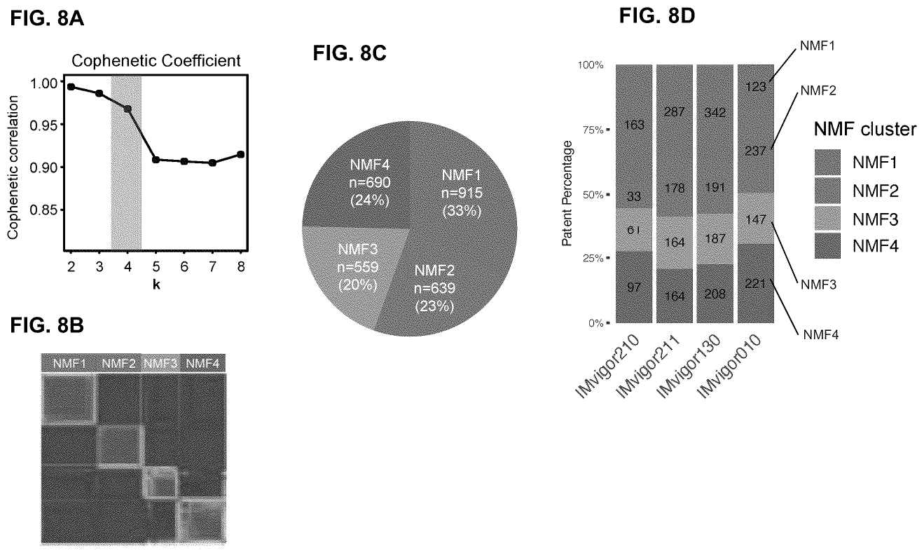

To identify transcriptionally-defined subgroups of patients in an unbiased way, we applied non-negative matrix factorization (NMF, Brunet et al. Proc Natl Acad Sci USA. 101:4164-4169 (2004)) on the RNAseq data from three of the four trials (IMvigor210/211/010), reserving IMvigor130 samples as an independent validation dataset. Based on cophenetic coefficient analysis, we identified four transcriptionally-defined clusters of tumors (FIGS. 8A and 8B). We then developed a machine learning based classifier trained on the discovery dataset and used it to predict the NMF categories in IMvigor130. Across the four trials, 915 (33%) NMF1, 639 (23%) NMF2, 559 (20%) NMF3 and 690 (24%) NMF4 tumors were identified (FIG. 8C). When analyzing NMF group distribution by trial, a significant difference was observed between trials (Chi-square p<0.001). NMF1 was enriched in metastatic settings (IMvigor210, 211 and 130), while NMF2 was enriched in MIBC (IMvigor010), suggesting a relationship between cancer stage and NMF group prevalence (FIG. 8D). Molecular subtype prevalence in IMvigor130 (validation set) was consistent with IMvigor211, highlighting the robustness of our classification in a large independent dataset.

iii. Clinical Outcome in UC Subtypes

We then analyzed the association between NMF subtypes and OS within and across treatment arms. We categorized treatment arms as atezolizumab-containing (atezolizumab monotherapy in IMvigor210, 211 and 130, and atezolizumab+chemotherapy in IMvigor130) or best standard-of-care (SOC, chemotherapy in IMvigor211 and 130, observation in IMvigor010). For OS associations, we only considered ctDNA+ patients in IMvigor010, whose disease progresses following surgical resection.

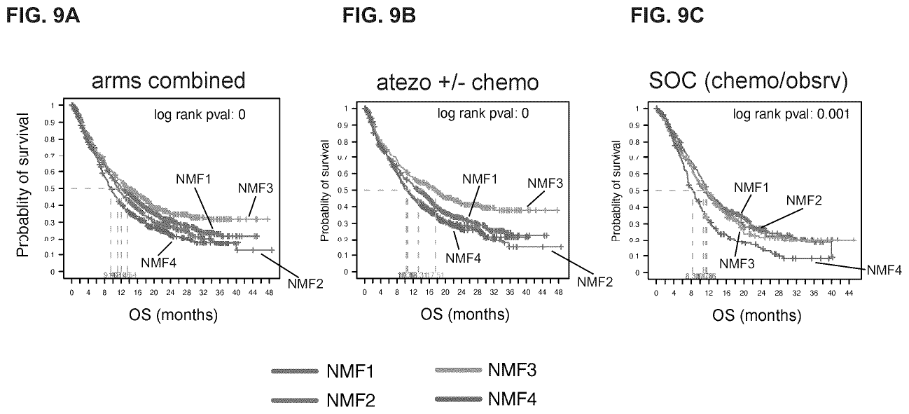

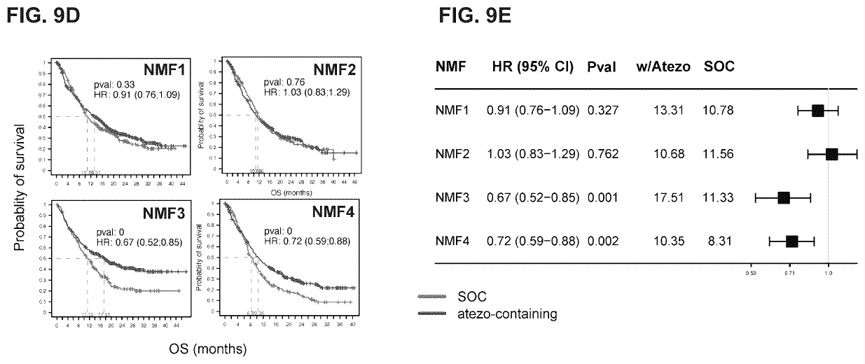

We first assessed the prognostic value of our molecular classification by comparing NMF subtypes in combined treatment arms. A significant association between OS and NMF subtypes was identified (p=2e-05), with NMF3 exhibiting the longest OS (median OS=13.5 months) and NMF4 exhibiting the shortest (median OS=9.5 months) (FIG. 9A). Splitting by treatment arm, NMF3 benefit was observed in patients treated with atezolizumab (median OS=17.5 months, p=2e-04), while NMF4 patients treated with SOC exhibited the shortest OS (median OS=8.31 months, p=8e-04) (FIGS. 9B and 9C). We then assessed the predictive value of NMF subtypes by comparing OS across arms within each group (FIGS. 9D and 9E). While no difference was observed between atezolizumab-containing and SOC arms in NMF1 and NMF2, significant OS benefit was observed in NMF3 (HR=0.67, p=8.7e-04, median OS: 17.5 vs. 11.3 months) and NMF4 (HR=0.72, p=2e-03, median OS: 10.3 vs. 8.3 months) in patients receiving PD-L1 blockade.

Overall, our transcriptional classification exhibited both prognostic and predictive clinical value, and identified UC patients from NMF3 and NMF4 (45% of evaluated population) subtypes as benefiting from PD-L1 blockade over SOC.

iv. Biological Characteristics of UC NMF Subtypes

To better understand the biological makeup of NMF subtypes, we combined known biomarkers, including PD-L1 on immune and tumor cells, CD8+ T cell infiltration phenotype, tumor mutation burden (TMB), with linear modeling on transcription data, pathway enrichment analysis, deconvolution of bulk RNAseq and digital pathology-derived human interpretable features (HIFs).

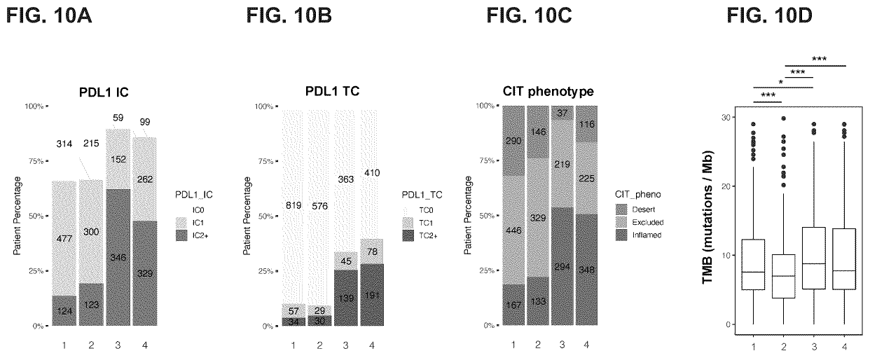

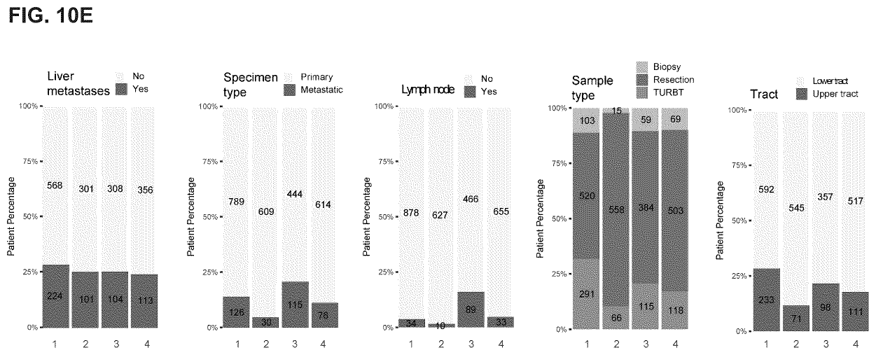

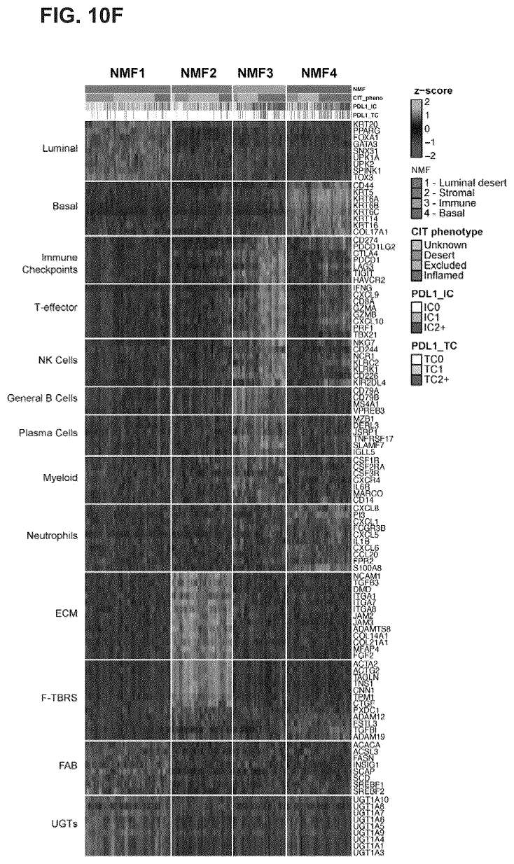

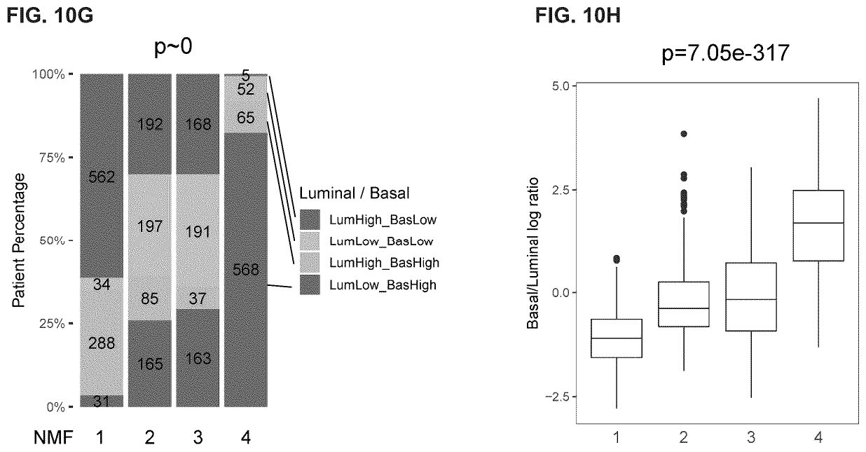

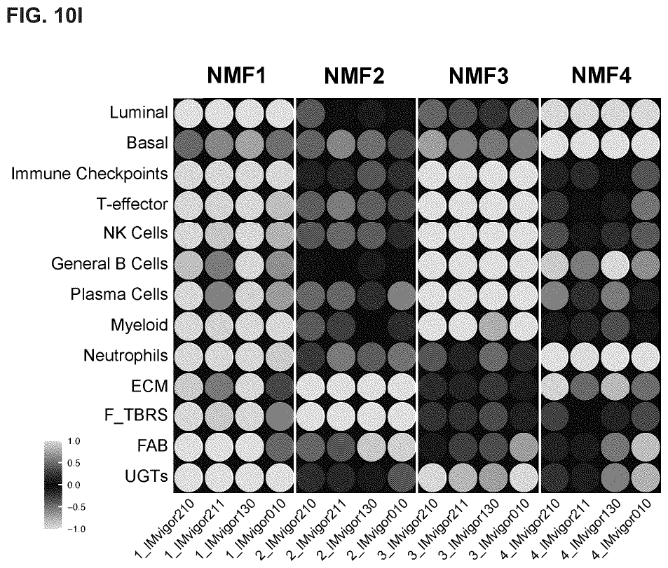

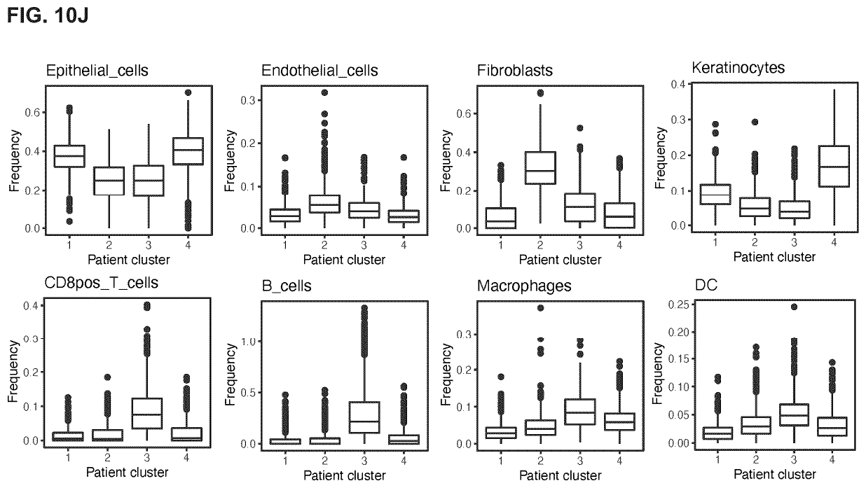

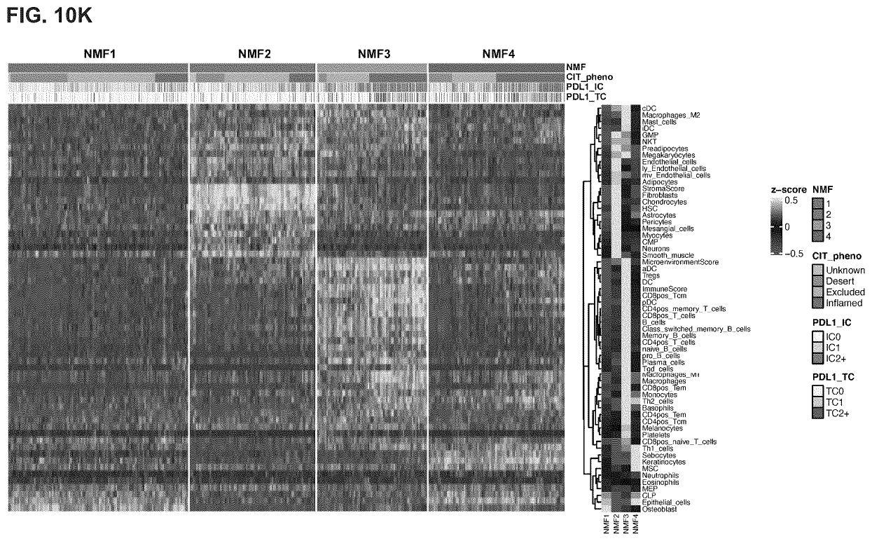

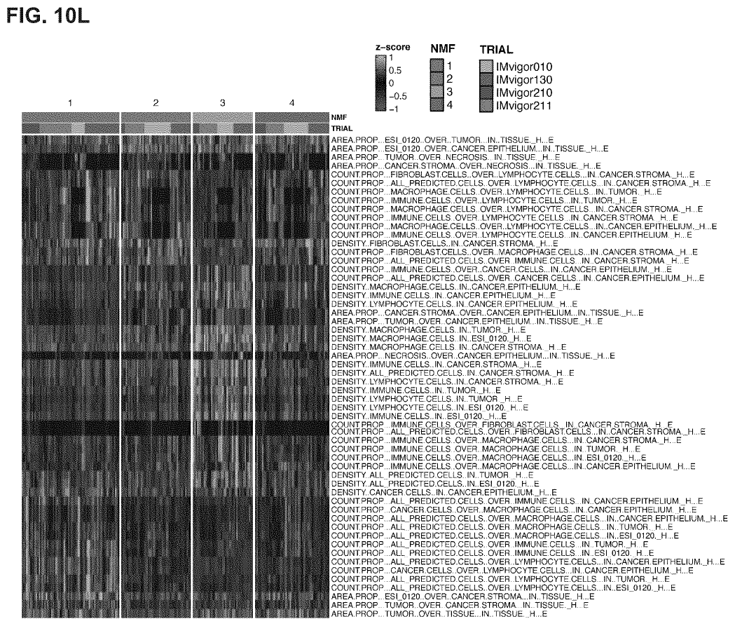

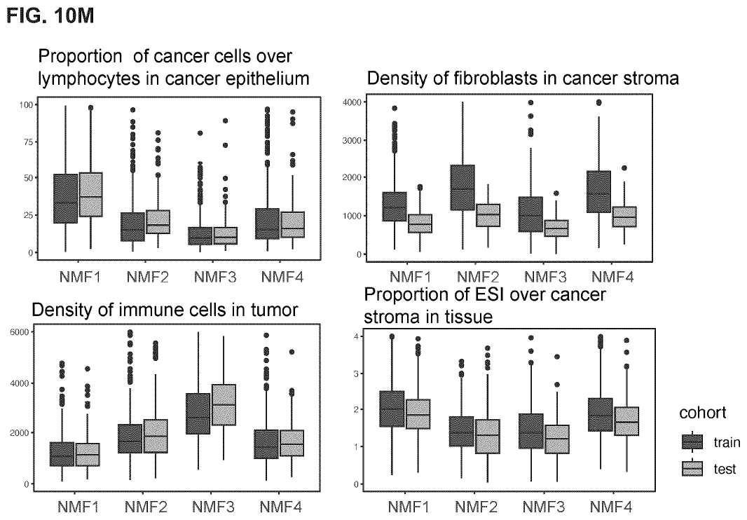

NMF3 and NMF4 exhibited high PD-L1 expression by IHC, both on immune (p=2.3e-106) (FIG. 10A) and tumor (p=3.6e-63) (FIG. 10B) cells. Using CD8 IHC, tumors can be categorized as i) inflamed, where CD8⁺ T cells have infiltrated the tumor epithelium; ii) excluded, where CD8⁺ T cells accumulate at the stromal barrier; iii) desert, where CD8⁺ T cells are absent from the tumor microenvironment. Both NMF3 and NMF4 exhibited a higher proportion of inflamed tumors, while NMF1 and NMF2 were enriched for desert and excluded tumors (FIG. 10C). TMB was significantly lower in NMF2 (Kruskal-Wallis p-value=1.47e-07; median TMB NMF1: 8.00 muts/mb; NMF2: 7.02 muts/mb; NMF3: 8.83 muts/mb; NMF4: 8.77 muts/mb) (FIG. 10D). We also checked whether specific clinical and tumor sampling features were driving molecular subgroups (FIG. 10E). No difference was observed in liver metastasis status between groups. While NMF2 was enriched for primary tumors, resections, and lower tract samples, and NMF3 was enriched for tumors sampled around lymph nodes, none of these parameters fully associated with specific molecular subtypes, suggesting the latter are independent of metastasis status and sampling location.

To further understand biological differences between NMF subtypes, we generated a heatmap using transcriptional signatures that recapitulate tumor, immune and stromal biologies (FIG. 10F). NMF1 tumors were enriched for a tumor-intrinsic luminal signature (KRT20), with low immune infiltrate, and increased metabolic signals, including programs related to fatty acid biosynthesis and uridine glucoronyl transferases (UGT), a family of enzymes involved in drug metabolism. NMF2 tumors were enriched for stromal signals, including a TGF-b-induced signature expressed in fibroblasts (F-TBRS) (Mariathasan et al. Nature. 554:544-548 (2018)), and an extracellular matrix (ECM) signature. NMF3 tumors were enriched for immune signals, including lymphoid (T/NK/B/Plasma cells) and myeloid signatures. Finally, NMF4 tumors were enriched for a tumor-intrinsic basal signature (KRT5, KRT6A/B/C, KRT14), with intermediate effector T cell infiltrate and low B/plasma cell signatures. We further dissected the luminal and basal signatures by dichotomizing the expression of each signature as high (>=median) or low (<median) and analyzing categorical distribution across NMF subtypes. NMF1 was enriched for Luminal^{high} Basal^{low} tumors, while NMF4 was enriched for Luminal^{low} Basal^{high} tumors (FIG. 10G). We confirmed this by generating a continuous Basal/Luminal signature ratio (FIG. 10H). NMF2 and NMF3 tumors seemed to be evenly split between basal and luminal phenotypes, with a molecular profile driven by their stromal and immune components respectively.

Biological pathways enriched in each NMF subtype were summarized at the signature, NMF subtype and clinical trial levels (FIG. 10I), highlighting the biological reproducibility of our classification scheme across studies. We further validated these observations by: a) deconvoluting transcriptional profiles to assess immune, stromal and tumor cell type enrichment by xCell (Aran et al. Genome Biol. 18:220 (2017)); b) comparing each NMF subtype against the other three through linear modeling, and conducting KEGG (Kanehisa and Goto. Nucleic Acids Res. 28:27-30 (2000)) pathway enrichment on differentially expressed genes. Based on xCell deconvolution (FIGS. 10J and 10K), NMF1 tumors were enriched in epithelial cells and osteoblasts. NMF2 tumors were enriched in fibroblasts, chondrocytes, endothelial cells, and a combined stromal score. NMF3 tumors were enriched for many immune populations, including CD4+ and CD8+ T cells, B cell subsets, plasma cells, macrophages, monocytes, and dendritic cells. NMF4 tumors were enriched for epithelial cells, keratinocytes, and sebocytes. KEGG analysis highlighted the enrichment of metabolic pathways in NMF1, extracellular matrix and angiogenic signals in NMF2, immune signals in NMF3 and proliferative and proinflammatory signals in NMF4. Based on these findings, we annotated NMF1 as luminal desert, NMF2 as stromal, NMF3 as immune and NMF4 as basal.

Finally, we applied digital pathology to assess whether automated hematoxylin and eosin (H&E) slide analysis could identify HIFs associated with molecular subtypes. Using machine learning with validation in an independent cohort, 59 unique HIFs were identified as significantly enriched in at least one NMF subtype (FIG. 10L). The proportion of cancer cells over lymphocytes in cancer epithelium was highest in NMF1, supporting the low immune infiltrate in this subtype. Conversely, the density of immune cells in tumors was highest in immune-enriched NMF3. The density of fibroblasts in cancer stroma was increased in both NMF2 and NMF4. Finally, the proportion of epithelial/stromal interface over cancer stroma was increased in both NMF1 and NMF4 (FIG. 10M). These data show that digital pathology can identify features of molecular subtypes on H&E slides, and could potentially be used to accelerate patient subtyping in clinical settings.

Overall, our classification reveals specific enrichment of tumor, immune and stromal compartments of the tumor microenvironment in each NMF subtype, supporting a tailored treatment approach in UC.

v. Comparison Against Existing Classifications

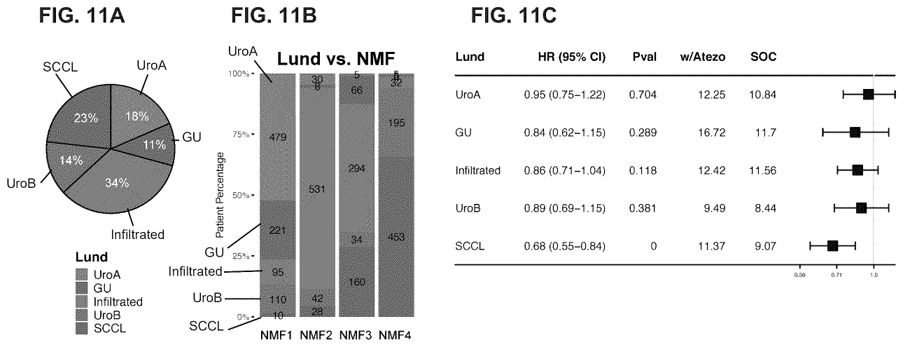

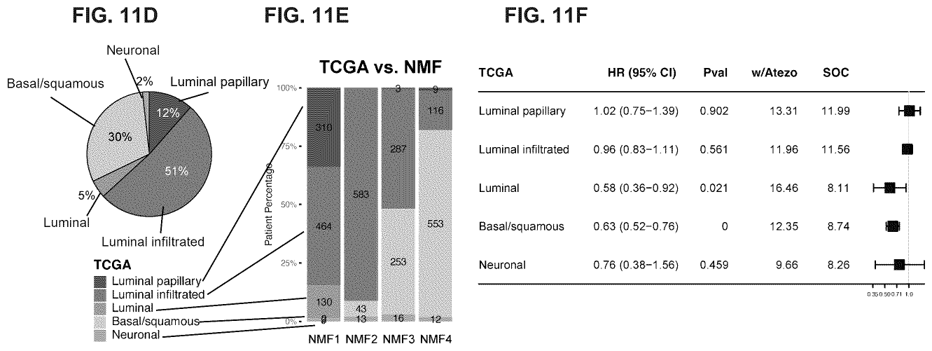

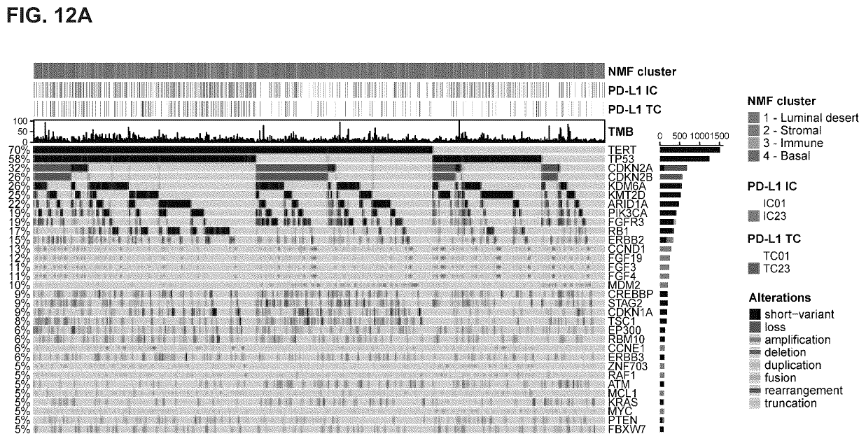

Several groups have previously defined transcriptional classifications in UC (Robertson et al. Cell. 171:540-556.e25 (2017), Sjödahl et al. Clin. Cancer Res. 18:3377-3386 (2012)), in smaller patient cohorts and in non-randomized settings. To compare and contrast our classification to these, we categorized our 2,803 samples according to the Lund (Urobasal A (UroA), genomically unstable (GU), Infiltrated, UroB, or squamous cell carcinoma-like (SCCL)) and the Cancer Genome Atlas (TCGA) (Luminal papillary, Luminal infiltrated, Luminal, Basal squamous, or Neuronal) classifications. We then analyzed category distribution across our NMF groups, as well as the association between Lund/TCGA subtypes and OS (FIGS. 11A-11F). NMF1 was enriched in Lund UroA and GU samples, and TCGA luminal papillary and luminal samples. NMF4 was enriched in Lund UroB and SCCL, corresponding to the TCGA basal/squamous group. NMF2 and NMF3 were enriched for Lund infiltrated and TCGA luminal infiltrated subtypes, with additional TCGA basal/squamous samples within NMF3. When looking at associations with outcome, only the Lund SCCL group (n=651/2,803, 23%, HR=0.68, p<0.01) showed significant benefit from the atezolizumab-containing arm over SOC. In the TCGA classification, both the Luminal (n=130/2,803, 5%, HR=0.58, p=0.02) and Basal squamous (n=851/2,803, 30%, HR=0.63, p<0.01) subtypes showed significant associations with outcome. Overall, while there is overlap between the various classifications, in particular for the basal and luminal papillary subtypes, our classification identifies OS benefit from atezolizumab-containing arms in both NMF3 (n=559/2,803, 20%) and NMF4 (n=690/2,803, 25%), for a combined prevalence of 45%, compared to TCGA (35%) and Lund (23%) classifications.

vi. Somatic Alterations in UC NMF Subtypes

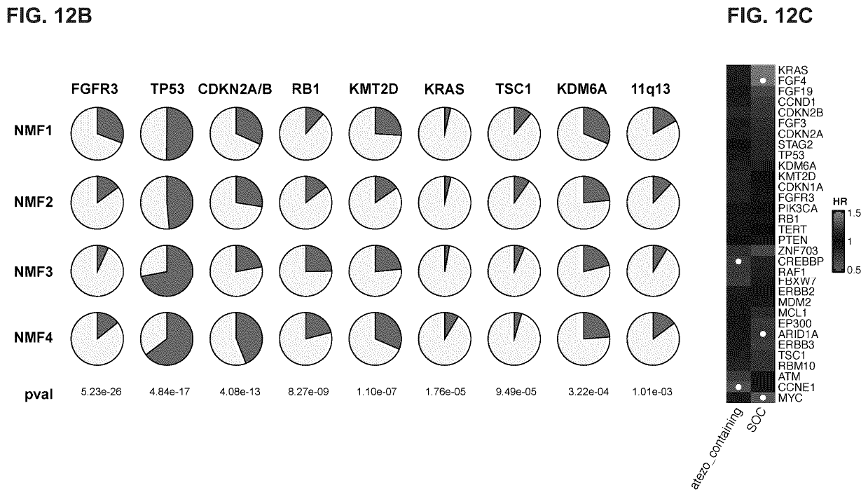

To complement transcriptomics, we analyzed somatic alterations in 2,168 patients using a targeted assay (FOUNDATIONONE®). Genes altered in at least 5% of patients were represented as an oncoprint in FIG. 12A. This analysis recapitulated somatic mutation profiles previously described in other UC cohorts (Robertson et al. Cell. 171:540-556.e25 (2017)) with alterations in TERT (70%), TP53 (58%), KDM6A (26%), KMT2D (25%), ARID1A (22%), PIK3CA (19%), FGFR3 (19%), RB1 (17%), and ERBB2 (15%). Loss-of-function alterations in the CDKN2A/B locus, mostly through copy-number loss, were observed in up to 32% of patients. Amplifying mutations in the chr11q13 band containing CCND1, FGF3, FGF4, and FGF19 were observed in 13% of patients.

We then asked whether transcriptionally-defined NMF subgroups exhibited enrichment in specific somatic alterations. NMF1 luminal desert tumors exhibited increased frequency of FGFR3 amplifying mutations (p=5.23e-26), and KDM6A loss-of-function (LOF) mutations (p=3.22e-04). NMF3 immune and NMF4 basal tumors were enriched for TP53 (p=4.84e-17) and RB1 (p=8.27e-09) LOF mutations. NMF4 basal tumors exhibited increased copy-number loss in the CDKN2A/B locus (p=4.08e-13), KMT2D (p=1.10e-07), and KRAS (p=1.76e-05) LOF mutations (FIG. 12B). This suggests NMF subgroups are partially enriched in tumor-intrinsic features, some of which could be targeted in the clinic, such as FGFR3 amplifications, or CDKN2A/B copy-number loss and TP53 LOF mutations.

Finally, we asked whether somatic alterations were associated with OS across clinical trial arms. We calculated hazard ratios in patients with somatically altered tumors vs. those with wild type tumors in atezolizumab-containing and SOC treatment arms in univariate analyses of genes identified in FIG. 12A. The results were summarized as a heatmap in FIG. 12C. We found few somatic alterations associated with outcome. Within the atezolizumab-containing arm, only CCNE1 and CREBBP single variant mutations were associated with improved OS. In the SOC arm, FGF4 alterations were associated with shorter OS, while alterations in ARID1A and MYC were associated with longer OS. Overall, this suggests that tumor DNA alterations alone are not sufficient to explain response to PD-(L) 1 blockade, and that the immune and stromal contextures need to be integrated into these analyses and considered when developing new combination therapies.

vii. Distinct Mechanisms of Response to Atezolizumab

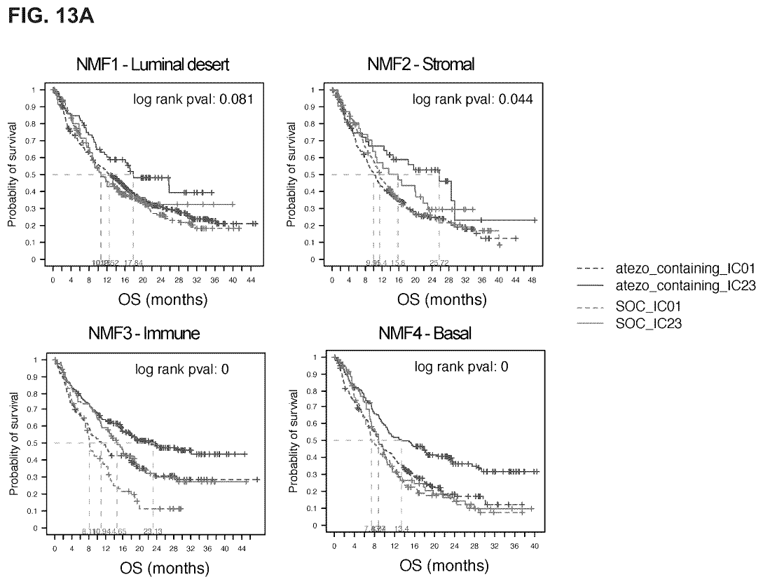

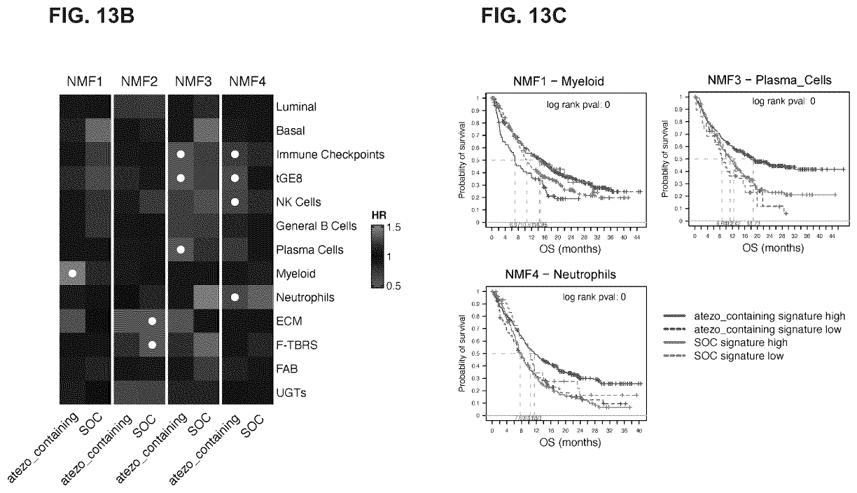

We then sought to better understand the molecular mechanisms underlying response within each molecular subtype. We first looked at the association of PD-L1 IC with OS. High PD-L1 IC expression benefited atezolizumab-treated patients in all subtypes (FIG. 13A). In NMF3, PD-L1 IC behaved as a prognostic biomarker, whereby both atezolizumab-containing (HR: 0.62, p=2.6e-03) and SOC (HR: 0.55, p=1.48e-03) arms benefited from high PD-L1 expression. A Cox proportional hazard model including an interaction term for arm and PD-L1 expression confirmed the prognostic value of PD-L1 IC in this group (interaction p>0.05). In NMF4, high PD-L1 IC was predictive of response in the atezolizumab-containing arm (HR: 0.60, p=9.4e-05), but not in the SOC arm (HR: 0.90, p=0.53). This suggests that the high immune infiltrate observed in NMF3 is also beneficial in SOC arms, and that PD-L1 blockade is necessary for benefit within the context of NMF4 basal tumors.

To identify additional transcriptional programs predictive of response/resistance to atezolizumab, we analyzed the association between signature expression and OS within each molecular subtype and each treatment arm (FIGS. 13B and 13C). Immune checkpoint and Teff signatures were predictive of response to atezolizumab in NMF3 and NMF4. The plasma cell signature was predictive of response to atezolizumab in NMF3 only, supporting our previous findings in NSCLC [REF Patil, Cancer Cell 2022]. In contrast, the neutrophil signature was predictive of response to atezolizumab in NMF4, and while associating with lower OS in patients treated with SOC. This suggests that blocking PD-L1 on neutrophils may help promote anti-tumor activity. Finally, the myeloid signature was associated with short OS in NMF1 patients treated with atezolizumab, suggesting a detrimental effect of myeloid cells in the context of low CD8+ T cell infiltration. Overall, these analyses reveal molecular subtype-specific programs that could be modulated by appropriate targeting agents combined with PD-L1 blockade to improve patient OS.

viii. Increased Neutrophil Infiltration in Basal UC Tumors

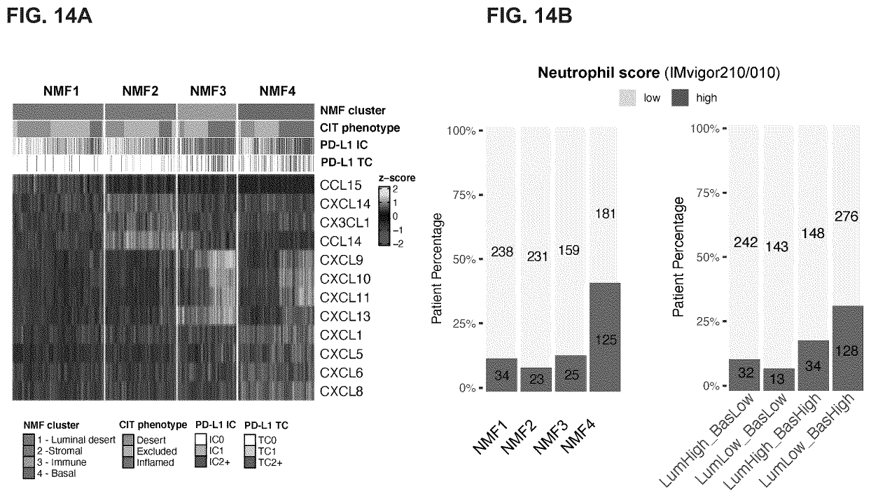

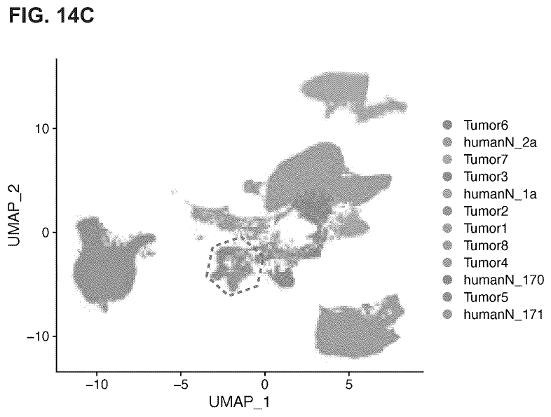

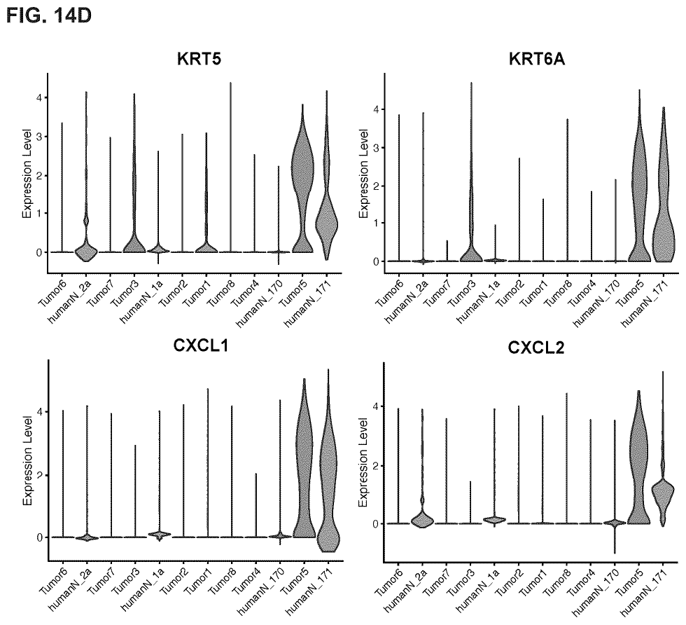

To further understand differences in immune recruitment patterns, we analyzed chemokine expression patterns across NMF subtypes. Lymphocyte chemoattractants CXCL9/10/11/13 were enriched in NMF3, while granulocyte chemoattractants CXCL1/5/6/8 were enriched in NMF4 (FIG. 14A). CCL14, CXCL14, CX3CL1, which are highly expressed by fibroblasts and endothelial cells, were overexpressed in stromal NMF2. Because granulocyte chemoattractants were overexpressed in NMF4, we assessed neutrophil presence by pathology on H&E slides in a subset of tumors from adjuvant, metastatic first-line and second-line UC (IMvigor010 and IMvigor210, n=1016). A significant enrichment in neutrophils (p=1.04e-24) was observed in NMF4 tumors, corresponding to tumors with high basal signature (FIG. 14B), supporting previous studies (Mandelli et al. Cells. 9:291 (2020)). To deconvolute the cellular source of granulocyte chemoattractant, we analyzed their expression in two publicly-available single cell RNAseq datasets from twelve patients with UC (Chen et al. Nat. Commun. 11:5077 (2020), Wang et al. Clin. Cancer Res. 27:4287-4300 (2021)). Focusing on the epithelial/tumor compartment (FIG. 14C), we identified two basal tumors with increased expression of KRT5 and KRT6A (FIG. 14D). These cells also expressed high levels of CXCL1 and CXCL2, suggesting that basal tumor cells intrinsically produce granulocyte chemoattractants. These data suggest that UC tumor intrinsic features can shape the immune microenvironment by recruiting inflammatory granulocytes, which may play a role in the pathogenicity of basal tumors.

### C. Conclusion

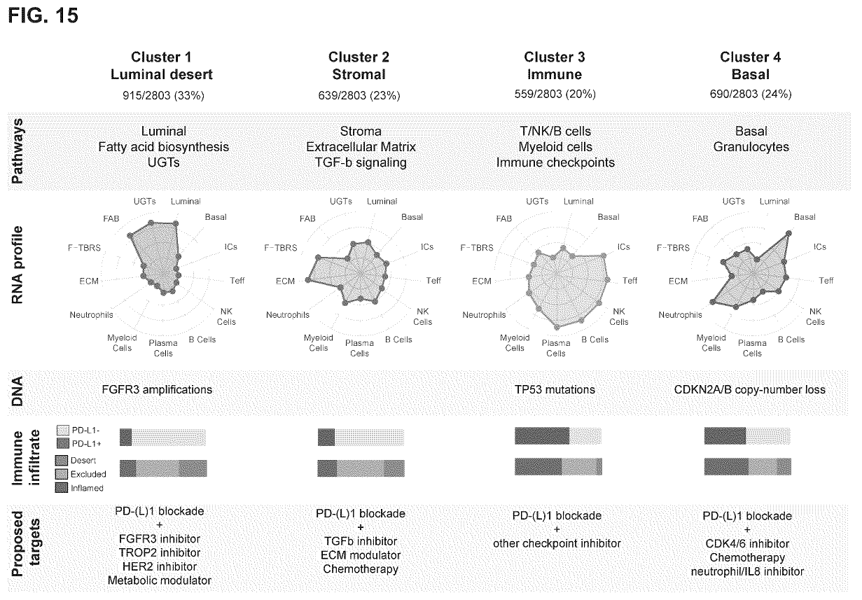

This study profiled 2,803 patients with urothelial carcinoma to provide the largest compendium of clinical and molecular data in this disease in the context of PD-L1 blockade. Applying machine learning on bulk pre-treatment tumor transcriptional profiles, agnostic of clinical information and treatment outcome, we identified four molecular subtypes of UC tumors (FIG. 15). We found that: a) these subtypes were driven by tumor-intrinsic (luminal vs. basal), immune and stromal programs, reflecting the balance between these three components of the tumor microenvironment; b) Two molecular subtypes, enriched either for lymphoid programs (especially B/plasma cells, NMF3) or basal tumor markers and neutrophil biology (NMF4), represented 45% of patients benefiting from atezolizumab over SOC; c) Somatic alterations showed limited biomarker value in the context of PD-L1 blockade, unless considered within NMF subtypes and as potential biomarkers for tumor-targeting agents; d) The mechanisms of response to PD-L1 blockade may be different between groups that show benefit from atezolizumab.

### OTHER EMBODIMENTS

Although the foregoing invention has been described in some detail by way of illustration and example for purposes of clarity of understanding, the descriptions and examples should not be construed as limiting the scope of the invention.

1. A method of treating urothelial cancer (UC) in a human patient, the method comprising:
(a) assaying mRNA in a tumor sample from the patient to provide a transcriptional profile of the patient's tumor;
(b) assigning the patient's tumor sample into one of the following four subtypes based on the transcriptional profile of the patient's tumor: luminal, stromal, immune, or basal,
wherein the method further comprises determining the mRNA expression level of one or more of the following gene signatures in the tumor sample from the patient:
(i) a luminal signature comprising keratin 20 (KRT20), peroxisome proliferator activated receptor gamma (PPARG), forkhead box A1 (FOXA1), GATA binding protein 3 (GATA3), sorting nexin 31 (SNX31), uroplakin 1A (UPK1A), uroplakin 2 (UPK2), serine peptidase inhibitor Kazal type 1 (SPINK1), and TOX high mobility group box family member 3 (TOX3);
(ii) a basal signature comprising cluster of differentiation 44 (CD44), keratin 5 (KRT5), keratin 6A (KRT6A), keratin 6B (KRT6B), keratin 6C (KRT6C), keratin 14 (KRT14), keratin 16 (KRT16), and collagen type XVII alpha 1 chain (COL17A1);
(iii) an immune checkpoint signature comprising cluster of differentiation 274 (CD274), programmed cell death 1 ligand 2 (PDCD1LG2), cytotoxic T-lymphocyte associated protein 4 (CTLA4), programmed cell death protein 1 (PDCD1), lymphocyte activating 3 (LAG3), T cell immunoreceptor with immunoglobulin (Ig) and immunoreceptor tyrosine-based inhibitory motif (ITIM) domains (TIGIT), and hepatitis A virus cellular receptor 2 (HAVCR2);
(iv) a T effector signature comprising interferon gamma (IFNG), C-X-C motif chemokine ligand 9 (CXCL9), cluster of differentiation 8A (CD8A), granzyme A (GZMA), granzyme B (GZMB), C-X-C motif chemokine ligand 10 (CXCL10), perforin 1 (PRF1), and T-Box transcription factor 21 (TBX21);
(v) a natural killer (NK) cell signature comprising natural killer cell granule protein 7 (NKG7), cluster of differentiation 244 (CD244), natural cytotoxicity triggering receptor 1 (NCR1), killer cell lectin like receptor C2 (KLRC2), killer cell lectin like receptor K1 (KLRK1), cluster of differentiation 266 (CD226), and killer cell immunoglobulin like receptor, two Ig domains and long cytoplasmic tail 4 (KIR2DL4);
(vi) a general B cell signature comprising cluster of differentiation 79A (CD79A), cluster of differentiation 79B (CD79B), membrane spanning 4-domains A1 (MS4A1), and V-set pre-B cell surrogate light chain 3 (VPREB3);
(vii) a plasma cell signature comprising marginal zone B and B1 cell specific protein (MZB1), derlin 3 (DERL3), junctional sarcoplasmic reticulum protein 1 (JSRP1), tumor necrosis factor (TNF) receptor superfamily member 17 (TNFRSF17), signaling lymphocytic activation molecule (SLAM) family member 7 (SLAMF7), and immunoglobulin lambda like polypeptide 5 (IGLL5);
(viii) a myeloid signature comprising colony stimulating factor 1 receptor (CSF1R), colony stimulating factor 2 receptor subunit alpha (CSF2RA), colony stimulating factor 3 receptor (CSF3R), C-X-C motif chemokine receptor 4 (CXCR4), interleukin 6 receptor (IL6R), macrophage receptor with collagenous structure (MARCO), and cluster of differentiation 14 (CD14);
(ix) a fibroblast transforming growth factor beta response signature (F-TBRS) comprising actin alpha 2, smooth muscle (ACTA2), actin gamma 2, smooth muscle (ACTG2), transgelin (TAGLN), tensin 1 (TNS1), calponin 1 (CNN1), tropomyosin 1 (TPM1), connective tissue growth factor (CTGF), PX domain containing 1 (PXDC1), ADAM metallopeptidase domain 12 (ADAM12), follistatin like 3 (FSTL3), transforming growth factor beta induced (TGFBI), and ADAM metallopeptidase domain 19 (ADAM19);
(x) a FAB signature comprising acetyl-CoA carboxylase alpha (ACACA), acyl-CoA synthetase long chain family member 3 (ACSL3), fatty acid synthase (FASN), insulin induced gene 1 (INSIG1), SREBF chaperone (SCAP), stearoyl-CoA desaturase (SCD), sterol regulatory element binding transcription factor 1 (SREBF1), and sterol regulatory element binding transcription factor 2 (SREBF2); and/or
(xi) a UDP glucuronosyltransferase signature (UGT) comprising UDP glucuronosyltransferase family 1 member A10 (UGT1A10), UDP glucuronosyltransferase family 1 member A8 (UGT1A8), UDP glucuronosyltransferase family 1 member A7 (UGT1A7), UDP glucuronosyltransferase family 1 member A6 (UGT1A6), UDP glucuronosyltransferase family 1 member A5 (UGT1A5), UDP glucuronosyltransferase family 1 member A9 (UGT1A9), UDP glucuronosyltransferase family 1 member A4 (UGT1A4), UDP glucuronosyltransferase family 1 member A1 (UGT1A1), and UDP glucuronosyltransferase family 1 member A3 (UGT1A3);
and
administering atezolizumab to the patient based on the UC subtype.

2.-8. (canceled)

9. The method of claim 1, wherein the patient's tumor sample is assigned into the luminal subtype, and the patient's tumor sample has an increased expression level, relative to a reference expression level, of the luminal signature,
optionally wherein the patient's tumor sample has an increased expression level, relative to a reference expression level, of the FAB signature and/or UGTs signature, and/or decreased expression levels, relative to reference expression levels, of the basal signature, the immune checkpoint signature, the T effector signature, the NK cell signature, the general B cell signature, the plasma cell signature, the myeloid signature, and/or the F-TBRS.

10. The method of claim 1, wherein the patient's tumor sample is assigned into the stromal subtype, and the patient's tumor sample has increased expression levels, relative to reference expression levels, of the F-TBRS,
optionally wherein the patient's tumor sample has decreased expression levels, relative to reference expression levels, of the basal signature, the immune checkpoint signature, the T effector signature, the NK cell signature, the plasma cell signature, and/or the FAB signature.

11. The method of claim 1, wherein the patient's tumor sample is assigned into the immune subtype, and the patient's tumor sample has increased expression levels, relative to reference expression levels, of the immune checkpoint signature, the T effector signature, the NK cell signature, the general B cell signature, the plasma cell signature, and/or the myeloid signature,
optionally wherein the patient's tumor sample has decreased expression levels, relative to reference expression levels, of the luminal signature, the basal signature, the F-TBRS, the FAB signature, and/or the UGTs signature.

12. The method of claim 1, wherein the patient's tumor sample is assigned into the basal subtype, and the patient's tumor sample has an increased expression level, relative to a reference expression level, of the basal signature,
optionally wherein the patient's tumor sample has decreased expression levels, relative to reference expression levels, of the luminal signature, the general B cell signature, the plasma cell signature, the FAB signature, and/or the UGTs signature.

13. The method of claim 1, wherein the reference expression level of a signature is the median Z-score of the signature in a population of patients having an UC.

14. The method of claim 1, wherein the patient's tumor sample is assigned into the immune subtype or the basal subtype, and the patient's tumor sample has (i) an increased expression level, relative to a reference expression level, of PD-L1 in tumor-infiltrating immune cells, tumor cells, or both; or (ii) an increased level, relative to a reference level, of cluster of differentiation 8 (CD8)+ T cell infiltration.

15. The method of claim 1, wherein the patient's tumor sample is assigned into the basal subtype, and the patient's tumor has an increased level, relative to a reference level, of granulocyte infiltration.

16.-21. (canceled)

22. The method of claim 1, wherein the patient's tumor sample is assigned into the immune subtype or basal subtype, and the method further comprises treating the patient by additionally administering to the patient one or more additional immunotherapy agents.

23. The method of claim 22, wherein the additional immunotherapy agent comprises a cluster of differentiation 28 (CD28) agonist, an OX40 agonist, a glucocorticoid-induced TNFR-related (GITR) agonist, a cluster of differentiation 137 (CD137) agonist, a cluster of differentiation 27 (CD27) agonist, an inducible T-cell costimulator (IC0S) agonist, a herpes virus entry mediator (HVEM) agonist, a natural killer group 2 member D (NKG2D) agonist, a MHC class I polypeptide-related sequence A (MICA) agonist, a natural killer cell receptor 2B4 agonist, a PD-1 axis binding antagonist, a CTLA4 antagonist, a TIM3 antagonist, a B and T lymphocyte associated (BTLA) antagonist, a V-domain Ig suppressor of T cell activation (VISTA) antagonist, a LAG3 antagonist, a B7-H4 antagonist, a cluster of differentiation 96 (CD96) antagonist, a TIGIT antagonist, a cluster of differentiation 226 (CD226) antagonist, a chemokine receptor 8 (CCR8) antagonist, a cancer vaccine, an adoptive cell therapy, or a combination thereof.

24. (canceled)

25. (canceled)

26. The method of claim 1, wherein the patient's tumor sample is assigned into the luminal subtype, and the method further comprises treating the patient by additionally administering to the patient one or more additional agents selected from a tyrosine kinase inhibitor (TKI), an FGFR3 antagonist, an anti-HER2 antibody drug conjugate (ADC), an anti-TROP2 ADC, or a combination thereof.

27. The method of claim 1, wherein the patient's tumor sample is assigned into the stromal subtype, and the method further comprises treating the patient by additionally administering to the patient one or more additional agents selected from a TKI, a TGF-β antagonist, a chemotherapeutic agent, or a combination thereof.

28.-43. (canceled)

44. The method of claim 1, wherein atezolizumab is administered as a monotherapy.

45. The method of 1, wherein atezolizumab is administered as an adjuvant therapy.

46. (canceled)

47. (canceled)

48. The method of claim 1, further comprising administering an additional therapeutic agent to the patient, wherein the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof.

49. The method of claim 48, wherein the additional therapeutic agent is an immunotherapy agent, a cytotoxic agent, a growth inhibitory agent, a stromal inhibitor, a metabolism inhibitor, a complement antagonist, a radiation therapy agent, an anti-angiogenic agent, or a combination thereof.

50. A kit for classifying a urothelial cancer (UC) in a human patient, the method comprising:
(a) assaying mRNA in a tumor sample from the patient to provide a transcriptional profile of the patient's tumor;
(b) assigning the patient's tumor sample into one of the following four subtypes based on the transcriptional profile of the patient's tumor: luminal, stromal, immune, or basal,
thereby classifying the UC.

51. The kit of claim 50, comprising:
(a) reagents for assaying mRNA in a tumor sample from the patient to provide a transcriptional profile of the patient's tumor; and
(b) instructions for assigning the patient's tumor sample into following four subtypes based on the transcriptional profile of the patient's tumor: luminal, stromal, immune, or basal.

52. An anti-cancer therapy for use in treating a UC in a human patient, wherein the UC in the patient has been classified by a method comprising:
(a) assaying mRNA in a tumor sample from the patient to provide a transcriptional profile of the patient's tumor; and
(b) assigning the patient's tumor sample into one of the following four subtypes based on the transcriptional profile of the patient's tumor: luminal, stromal, immune, or basal,
thereby classifying the UC.

53.-57. (canceled)
